(19) United States
(12) Patent Application Publication
(10) Pub. No.: US 2020/0216493 A1
(43) Pub. Date: Jul. 9, 2020

(54) ANTIMICROBIAL PEPTIDES

(71) Applicant: FERRING B.V., Hoofddorp (NL)

(72) Inventors: Antoine HENNINOT, San Diego, CA (US); Daniel William CARNEY, San Diego, CA (US)

(73) Assignee: FERRING B.V., Hoofddorp (NL)

(21) Appl. No.: 16/468,900

(22) PCT Filed: Dec. 13, 2017
(86) PCT No.: PCT/EP2017/082697

§ 371 Date: Jun. 12, 2019

Related U.S. Application Data
(60) Provisional application No. 62/433,567, filed on Dec. 13, 2016.

(51) Int. Cl. C07K 7/06; C07K 7/56; A61P 31/04

(52) U.S. Cl. CPC C07K 7/06; A61K 38/00; A61P 31/04; C07K 7/56

(57) ABSTRACT

This disclosure relates to compounds of formula (I) or a pharmaceutically acceptable salt thereof: Formula (I), in which n, R₁, R₁′, R₂, R₃, R₄, R₄′, and R₅-R₁₄ are defined in the specification. The compounds of formula (I) can be used to treat bacterial infection.

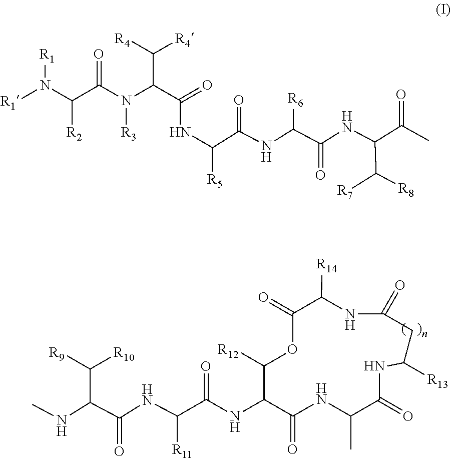

### CROSS-REFERENCE TO RELATED APPLICATION

This application claims priority under 35 U.S.C. § 119(e) to U.S. Provisional Application No. 62/433,567 filed Dec. 13, 2016, the disclosure of which is incorporated herein in its entirety.

### TECHNICAL FIELD

This disclosure relates to antimicrobial peptides, as well as related compositions and methods.

### BACKGROUND

According to the Centers for Disease Control (CDC) 2013 report on antibiotic resistance threats, antimicrobial resistance is one of our most serious health threats. As such, new antibiotics are needed to combat bacterial infections.

### SUMMARY

In one aspect, this disclosure features a compound of formula (I) or a pharmaceutically acceptable salt thereof:

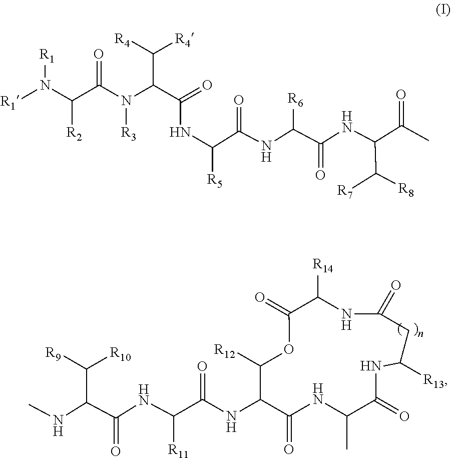

in which n is 0 or 1; each of R₁ and R₁′, independently, is H, C₁-C₆ alkyl, C(O)-Rₐ, or C(O)O—Rₐ, in which Rₐ is C₁-C₆ alkyl optionally substituted with NH₂, aryl, or heteroaryl; R₂ is C₁-C₆ alkyl optionally substituted with aryl or heteroaryl, in which the aryl or heteroaryl group is optionally substituted with halo, NH₂, C₁-C₆ alkyl, or C₁-C₆ alkoxy; R₃ is H, C₁-C₆ alkyl, or C(O)—R_{b}, in which R_{b} is C₁-C₆ alkyl; each of R₄ and R₄′, independently, is H, C₁-C₆ alkyl, aryl, or heteroaryl; R₅ is C₁-C₆ alkyl optionally substituted with OH, NH—R_{c}, aryl, or heteroaryl, or aryl optionally substituted with C₁-C₆ alkyl, in which R_{c}′ is H, C(O)O—R_{c}′, or —SO₂-phenyl optionally substituted with C₁-C₆ alkyl, R_{c}′ being C₁-C₆ alkyl or C₁-C₆ alkenyl; R₆ is C₁-C₆ alkyl optionally substituted with C(O)—NH(Rd), NH(R_{d}), NHC(O)—R_{d}, aryl, or heteroaryl, in which each R_{d}, independently, is H, aryl, heteroaryl, or C₁-C₆ alkyl optionally substituted with aryl; R₇ is H, C₁-C₆ alkyl, aryl, or heteroaryl; R₈ is H, C₁-C₆ alkyl, aryl, or heteroaryl; R₉ is H, C₁-C₆ alkyl, aryl, or heteroaryl; R₁₀ is H, C₁-C₆ alkyl, aryl, or heteroaryl; R₁₁ is C₁-C₆ alkyl optionally substituted with OH, NH₂, aryl, or heteroaryl; R₁₂ is H or C₁-C₆ alkyl; R₁₃ is C₁-C₆ alkyl optionally substituted with heteroaryl, NH(=NH)NH(Rₑ), NH(=O)NH(Rₑ), N(Rₑ)₂, N(Rₑ)₃⁺, COORₑ, COO—NH(CH₂)₂N(Rₑ)₂, or aryl optionally substituted with NH(=NH)NH(Rₑ), in which each Re, independently, is H, NO₂, C₁-C₆ alkyl optionally substituted with aryl (e.g., phenyl), or C(O)—Re′, Re′ being C₁-C₆ alkyl; and R₁₄ is C₁-C₆ alkyl optionally substituted with NH₂; provided that the compound is not

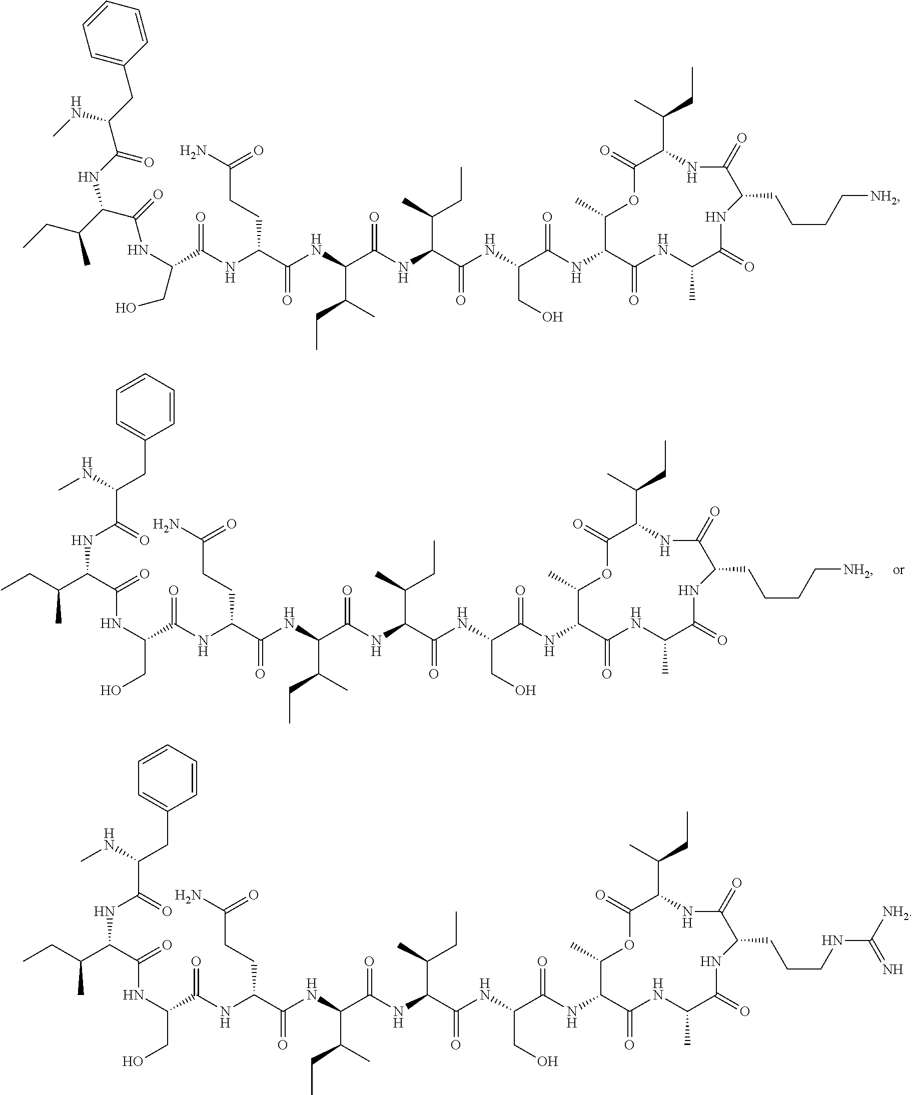

In another aspect, this disclosure features a pharmaceutical composition that includes a compound of formula (I) described herein and a pharmaceutically acceptable carrier.

In still another aspect, this disclosure features a method of treating bacterial infection that includes administering to a patient in need thereof an effective amount of the pharmaceutical composition described herein.

In still another aspect, this disclosure features the compounds or pharmaceutical compositions described herein for use as a medicament.

In still another aspect, this disclosure features the compounds or pharmaceutical compositions described herein for use in a method of treating bacterial infection.

In still another aspect, this disclosure features the use of the compounds disclosed herein in the manufacture of a medicament for the treatment of bacterial infection.

Other features, objects, and advantages will be apparent from the description and the claims.

### DETAILED DESCRIPTION

This disclosure generally relates to peptides (e.g., depsipeptides) that can be used for treating bacterial infection. In particular, this disclosure is based on the unexpected discovery that certain peptides can be used effectively for treating infection by gram-positive bacteria (e.g., Clostridium difficile or Staphylococcus aureus) and gram-negative bacteria (e.g., Escherichia coli), including their drug resistant strains. In addition, these peptides can be synthesized with a relatively high synthetic yield. In certain embodiments, the antimicrobial peptides described herein can have improved selectivity for gram-positive bacteria versus gram-negative bacteria. In certain embodiments, the antimicrobial peptides can have improved potency and selectivity for C. difficile versus other bacteria. In some embodiments, the antimicrobial peptides can have both high potency and low cytotoxicity when treating a bacterial infection. In certain embodiments, the antimicrobial peptides can have improved pharmacokinetic properties and/or biophysical properties (such as solubility and stability).

In some embodiments, the antimicrobial peptides described herein are those of formula (I) or a pharmaceutically acceptable salt thereof:

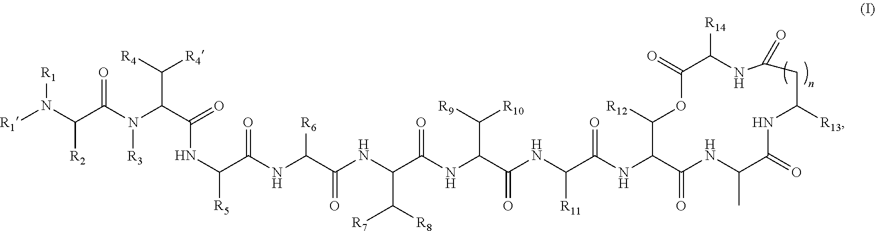

in which n is 0 or 1; each of R₁ and R₁′, independently, is H, C₁-C₆ alkyl, C(O)—Ra, or C(O)O—Ra, in which Rₐ is C₁-C₆ alkyl optionally substituted with NH₂, aryl, or heteroaryl; R₂ is C₁-C₆ alkyl optionally substituted with aryl or heteroaryl, in which the aryl or heteroaryl group is optionally substituted with halo, NH₂, C₁-C₆ alkyl, or C₁-C₆ alkoxy; R₃ is H, C₁-C₆ alkyl, or C(O)—R_{b}, in which R_{b} is C₁-C₆ alkyl; each of R₄ and R₄′, independently, is H, C₁-C₆ alkyl, aryl, or heteroaryl; R₅ is C₁-C₆ alkyl optionally substituted with OH, NH—R_{c}, aryl, or heteroaryl, or aryl optionally substituted with C₁-C₆ alkyl, in which R_{c} is H, C(O)O—R_{c}′, or —SO₂-phenyl optionally substituted with C₁-C₆ alkyl, R_{c}′ being C₁-C₆ alkyl or C₁-C₆ alkenyl; R₆ is C₁-C₆ alkyl optionally substituted with C(O)—NH(R_{d}), NH(R_{d}), NHC(O)—R_{d}, aryl, or heteroaryl, in which each R_{d}, independently, is H, aryl, heteroaryl, or C₁-C₆ alkyl optionally substituted with aryl; R₇ is H, C₁-C₆ alkyl, aryl, or heteroaryl; R₈ is H, C₁-C₆ alkyl, aryl, or heteroaryl; R₉ is H, C₁-C₆ alkyl, aryl, or heteroaryl; R₁₀ is H, C₁-C₆ alkyl, aryl, or heteroaryl; R₁₁ is C₁-C₆ alkyl optionally substituted with OH, NH₂, aryl, or heteroaryl; R₁₂ is H or C₁-C₆ alkyl; R₁₃ is C₁-C₆ alkyl optionally substituted with heteroaryl, NH(=NH)NH(Rₑ), NH(=O)NH(Rₑ), N(Rₑ)₂, N(Rₑ)₃⁺, COORₑ, COO—H(CH₂)₂N(Rₑ)₂, or aryl optionally substituted with NH(=NH)NH(Rₑ), in which each Rₑ, independently, is H, NO₂, C₁-C₆ alkyl optionally substituted with aryl, or C(O)—Rₑ′, Re′ being C₁-C₆ alkyl; and R₁₄ is C₁-C₆ alkyl optionally substituted with NH₂; provided that the compound is not

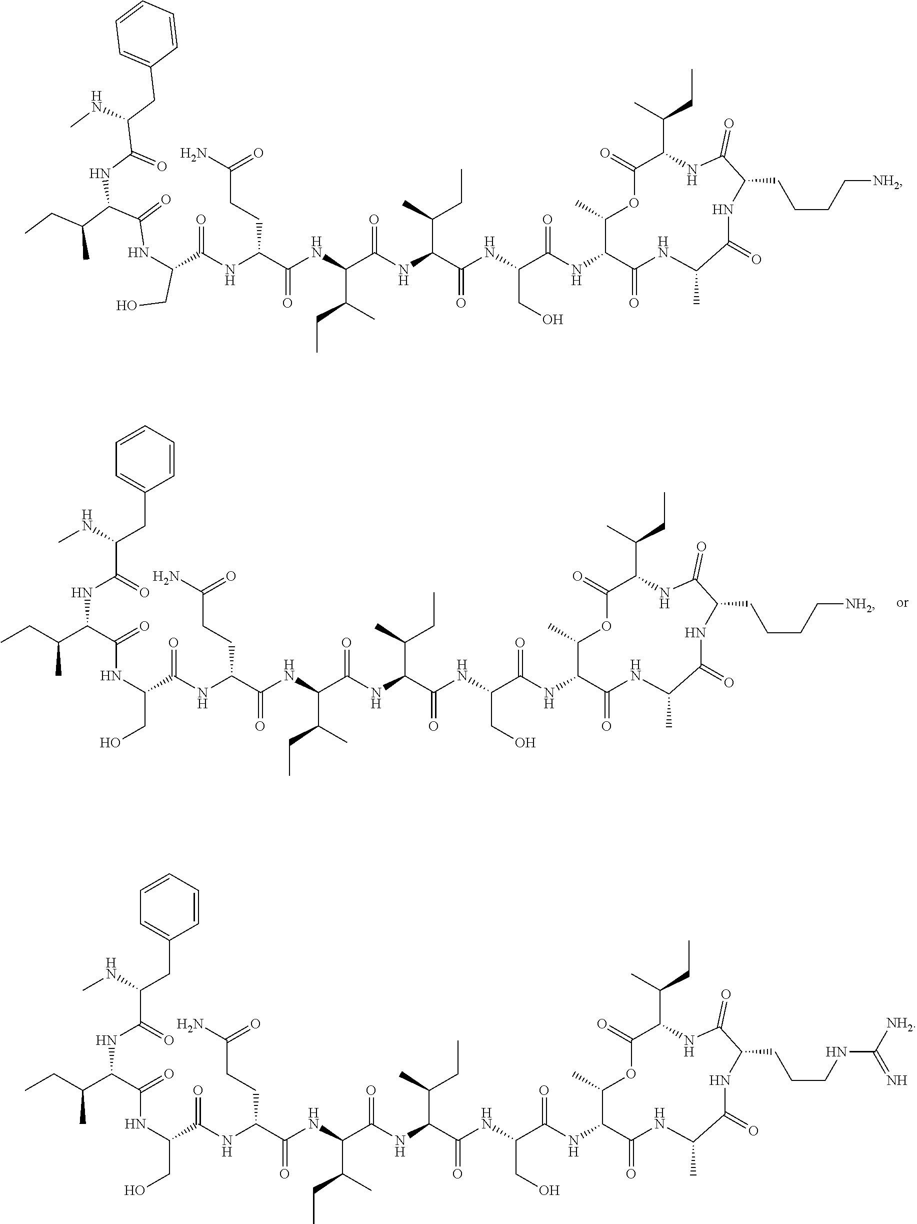

The term “alkyl” refers to a saturated, linear or branched hydrocarbon moiety, such as —CH₃ or —CH(CH₃)₂. The term “alkoxy” refers to a saturated, linear or branched hydrocarbon moiety covalently bonded with an oxygen radical, such as —OCH₃ or —OCH(CH₃)_{2.} The term “alkenyl” refers to a linear or branched hydrocarbon moiety containing a carbon-carbon double bond, such as —CH₂—CH=CH₂ or —CH=C(CH₃)₂. The term “aryl” refers to a hydrocarbon moiety having one or more aromatic rings. Examples of aryl moieties include phenyl (Ph), phenylene, naphthyl, naphthylene, pyrenyl, anthryl, and phenanthryl. The term “heteroaryl” refers to a moiety having one or more aromatic rings that contain at least one heteroatom (e.g., N, O, or S). Examples of heteroaryl moieties include furyl, furylene, fluorenyl, pyrrolyl, thienyl, oxazolyl, imidazolyl, thiazolyl, pyridinyl, pyrimidinyl, quinazolinyl, quinolyl, isoquinolyl and indolyl.

In some embodiments, the antimicrobial peptides described herein are those of formula (II) or a pharmaceutically acceptable salt thereof:

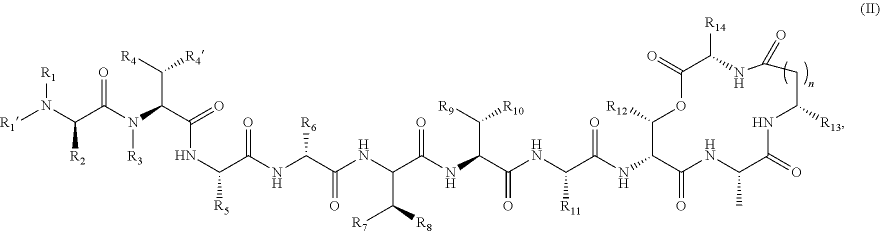

in which n, R₁, R₁′, R₂, R₃, R₄, R₄′, and R₅-R₁₄ can be the same as those described in formula (I) above.

In some embodiments, n in formulas (I) and (II) is 0.

In some embodiments, R₁ in formulas (I) and (II) is H or C₁-C₆ alkyl, and R₁′ is H.

In some embodiments, R₂ in formulas (I) and (II) is C₁-C₆ alkyl optionally substituted with aryl or heteroaryl, in which the aryl or heteroaryl group is substituted with halo, NH₂, C₁-C₆ alkyl, or C₁-C₆ alkoxy. In some embodiments, R₂ in formulas (I) and (II) is C₁-C₆ alkyl substituted with phenyl, in which the phenyl group is optionally substituted with halo.

In some embodiments, R₃ in formulas (I) and (II) is H or C(O)—R_{b}, in which R_{b} is C₁-C₆ alkyl.

In some embodiments, R₄ in formulas (I) and (II) is H, C₁-C₆ alkyl, aryl, or heteroaryl; and R₄′ is C₁-C₆ alkyl, aryl, or heteroaryl. In some embodiments, R₄ is H, C₁-C₆ alkyl, or heteroaryl, and R₄′ is H or C₁-C₆ alkyl.

In some embodiments, R₅ in formulas (I) and (II) is methyl optionally substituted with NH—R_{c}, aryl, or heteroaryl, C₂-C₆ alkyl optionally substituted with OH, NH—R_{c}, aryl, or heteroaryl, or aryl optionally substituted with C₁-C₆ alkyl, in which R_{c} is H, C(O)O—R_{c}′, or —SO₂-phenyl optionally substituted with C₁-C₆ alkyl, R_{c}′ being C₁-C₆ alkyl or C₁-C₆ alkenyl. In some embodiments, R₅ in formulas (I) and (II) is aryl, or C₁-C₆ alkyl substituted with OH, NH₂, or heteroaryl.

In some embodiments, R₆ in formulas (I) and (II) is C₁-C₆ alkyl substituted with C(O)NH₂.

In some embodiments, each of R₇, R₈, R₉, R₁₀, R₁₂, and R₁₄ in formulas (I) and (II) is C₁-C₆ alkyl.

In some embodiments, R₁₁ in formulas (I) and (II) is C₁-C₆ alkyl substituted with OH.

In some embodiments, R₁₃ in formulas (I) and (II) is C₁, C₂, or C₄-C₆ alkyl optionally substituted with heteroaryl, NH(=NH)NH(Rₑ), NH(=O)NH(Rₑ), COORₑ, COO—NH(CH₂)₂N(Rₑ)₂, or aryl optionally substituted with NH(=NH)NH(Rₑ), in which each Rₑ, independently, is H, NO₂, C₁-C₆ alkyl optionally substituted with aryl, or C(O)—Rₑ′, Rₑ′ being C₁-C₆ alkyl. In some embodiments, R₁₃ in formulas (I) and (II) is C₁, C₂, C₅ or C₆ alkyl optionally substituted with heteroaryl, NH(=NH)NH(Rₑ), NH(=O)NH(Rₑ), N(Rₑ)₂, N(Rₑ)₃⁺, COORₑ, COO—NH(CH₂)₂N(Rₑ)₂, or aryl optionally substituted with NH(=NH)NH(Rₑ), in which each Rₑ, independently, is H, NO₂, C₁-C₆ alkyl, or C(O)—Rₑ′, Rₑ′ being C₁-C₆ alkyl. In some embodiments, R₁₃ in formulas (I) and (II) is C₁-C₆ alkyl substituted with NH(=NH)NH(Rₑ) or N(Rₑ)₂, in which each Rₑ, independently, is H or C₁-C₆ alkyl.

A first subset of compounds of formula (I) or (II) are those in which n is 0 or 1; each of R₁ and R₁′, independently, is H, C₁-C₆ alkyl, C(O)—Rₐ, or C(O)O—Rₐ, in which Rₐ is C₁-C₆ alkyl optionally substituted with NH₂, aryl, or heteroaryl; R₂ is C₁-C₆ alkyl optionally substituted with aryl or heteroaryl, in which the aryl or heteroaryl group is optionally substituted with halo, NH₂, C₁-C₆ alkyl, or C₁-C₆ alkoxy; R₃ is H, C₁-C₆ alkyl, or C(O)-Rb, in which R_{b} is C₁-C₆ alkyl; each of R₄ and R₄′, independently, is H, C₁-C₆ alkyl, aryl, or heteroaryl; R₅ is C₁-C₆ alkyl optionally substituted with OH, NH—R_{c}, aryl, or heteroaryl, or aryl optionally substituted with C₁-C₆ alkyl, in which R_{c} is H, C(O)O—R_{c}′, or —SO₂-phenyl optionally substituted with C₁-C₆ alkyl, R_{c}′ being C₁-C₆ alkyl or C₁-C₆ alkenyl; R₆ is C₁-C₆ alkyl optionally substituted with C(O)—NH(R_{d}), NH(R_{d}), NHC(O)—R_{d}, aryl, or heteroaryl, in which each R_{d}, independently, is H, aryl, heteroaryl, or C₁-C₆ alkyl optionally substituted with aryl; R₇ is H, C₁-C₆ alkyl, aryl, or heteroaryl; R₈ is H, C₁-C₆ alkyl, aryl, or heteroaryl; R₉ is H, C₁-C₆ alkyl, aryl, or heteroaryl; R₁₀ is H, C₁-C₆ alkyl, aryl, or heteroaryl; R₁₁ is C₁-C₆ alkyl optionally substituted with OH, NH₂, aryl, or heteroaryl; R₁₂ is H or C₁-C₆ alkyl; R₁₃ is C₁, C₂, or C₄-C₆ alkyl optionally substituted with aryl, heteroaryl, NH(=NH)NH(Rₑ), NH(=O)NH(Re), COORe, CO—NH(CH₂)₂N(Rₑ)₂, in which each Rₑ, independently, is H, NO₂, C₁-C₆ alkyl, or C(O)—Rₑ′, Rₑ′ being C₁-C₆ alkyl; and R₁₄ is C₁-C₆ alkyl optionally substituted with NH₂.

In such embodiments, n can be 0; R₁ can be C₁-C₆ alkyl; R₁′ can be H; R₂ can be C₁-C₆ alkyl substituted with phenyl; R₃ can be H; R₄ can be C₁-C₆ alkyl; R₄′ can be C₁-C₆ alkyl; R₅ can be C₁-C₆ alkyl substituted with OH, NH₂, or heteroaryl; R₆ can be C₁-C₆ alkyl substituted with C(O)NH₂; each of R₇, R₈, R₉, R₁₀ , R₁₂, and R₁₄ can be C₁-C₆ alkyl; R₁₁ can be C₁-C₆ alkyl substituted with OH; and R₁₃ can be C₁-C₆ alkyl substituted with NH(=NH)NH(Rₑ), in which Rₑ can be H or C₁-C₆ alkyl. Examples of such compounds include

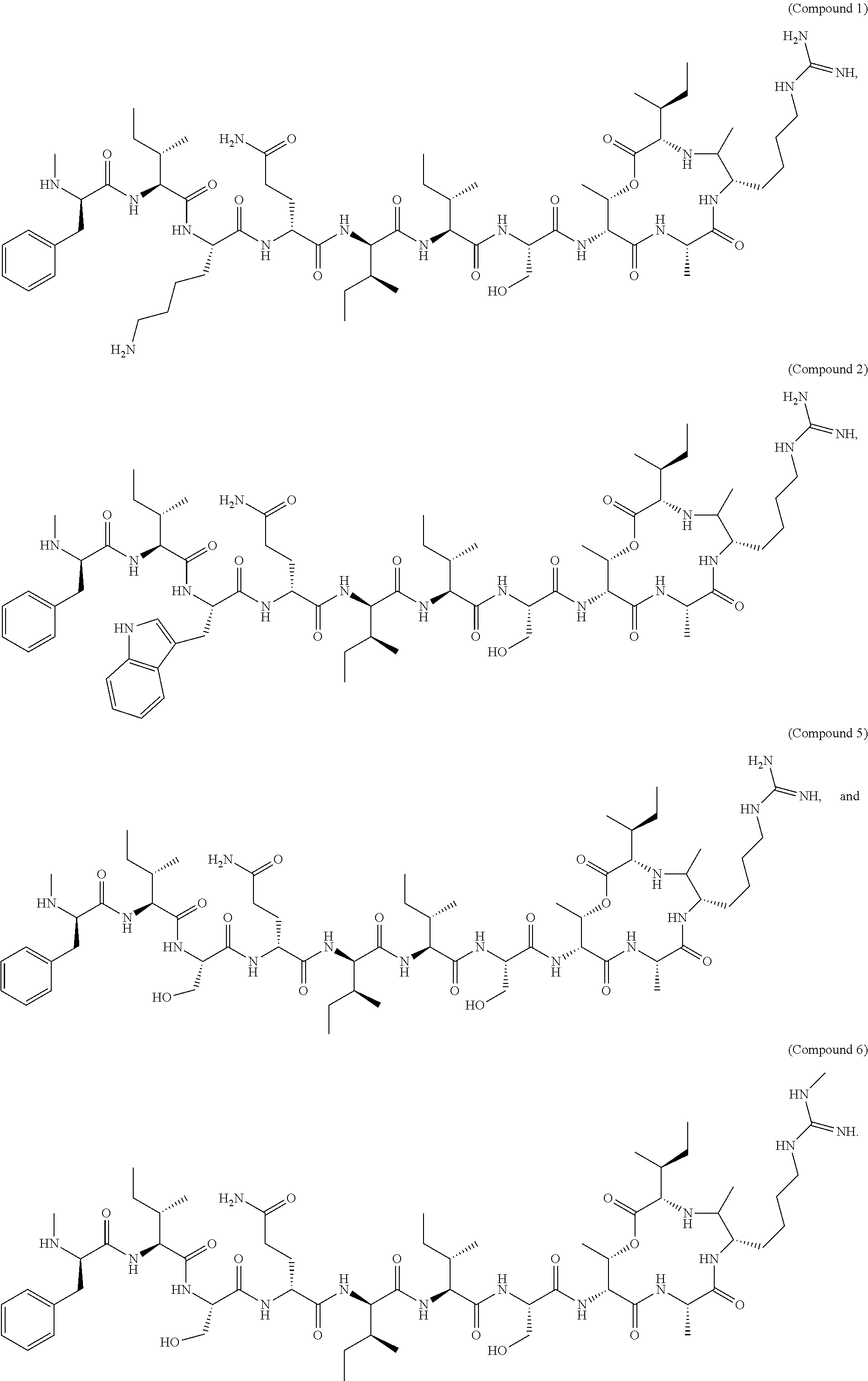

A second subset of compounds of formula (I) or (II) are those in which n is 0 or 1; each of R₁ and R₁′, independently, is H, C₁-C₆ alkyl, C(O)—Rₐ, or C(O)O—Rₐ, in which Rₐ is C₁-C₆ alkyl optionally substituted with NH₂, aryl, or heteroaryl; R₂ is C₁-C₆ alkyl optionally substituted with aryl or heteroaryl, in which the aryl or heteroaryl group is optionally substituted with halo, NH₂, C₁-C₆ alkyl, or C₁-C₆ alkoxy; R₃ is H, C₁-C₆ alkyl, or C(O)—R_{b}, in which R_{b} is C₁-C₆ alkyl; each of R₄ and R₄′, independently, is H, C₁-C₆ alkyl, aryl, or heteroaryl; R₅ is methyl optionally substituted with NH—R_{c}, aryl, or heteroaryl, C₂-C₆ alkyl optionally substituted with OH, NH—R_{c}, aryl, or heteroaryl, or aryl optionally substituted with C₁-C₆ alkyl, in which R_{c} is H, C(O)O—R_{c}′, or —SO₂-phenyl optionally substituted with C₁-C₆ alkyl, R_{c}′ being C₁-C₆ alkyl or C₁-C₆ alkenyl; R₆ is C₁-C₆ alkyl optionally substituted with C(O)—NH(R_{d}), NH(Rd), NHC(O)—R_{d}, aryl, or heteroaryl, in which each R_{d}, independently, is H, aryl, heteroaryl, or C₁-C₆ alkyl optionally substituted with aryl; R₇ is H, C₁-C₆ alkyl, aryl, or heteroaryl; R₈ is H, C₁-C₆ alkyl, aryl, or heteroaryl; R₉ is H, C₁-C₆ alkyl, aryl, or heteroaryl; R₁₀ is H, C₁-C₆ alkyl, aryl, or heteroaryl; R₁₁ is C₁-C₆ alkyl optionally substituted with OH, NH₂, aryl, or heteroaryl; R₁₂ is H or C₁-C₆ alkyl; R13 is C₁-C₆ alkyl optionally substituted with aryl, heteroaryl, NH(=NH)NH(Rₑ), NH(=O)NH(Rₑ), N(Rₑ)₂, N(Rₑ)₃⁺, COORₑ, or CO—NH(CH₂)₂N(Rₑ)₂, in which each Rₑ, independently, is H, NO₂, C₁-C₆ alkyl, or C(O)—Rₑ′, Rₑ′ being C₁-C₆ alkyl; and R₁₄ is C₁-C₆ alkyl optionally substituted with NH₂.

In such embodiments, n can be 0; R₁ can be C₁-C₆ alkyl; R₁′ can be H; R₂ can be C₁-C₆ alkyl substituted with phenyl; R₃ can be H; R₄ can be C₁-C₆ alkyl; R₄′ can be C₁-C₆ alkyl; R₅ can be aryl, methyl substituted with aryl or heteroaryl, or C₂-C6 alkyl substituted with NH₂; R₆ can be C₁-C₆ alkyl substituted with C(O)NH₂; each of R₇, R₈, R₉, R₁₀, R₁₂, and R₁₀ can be C₁-C₆ alkyl; R₁₁ can be C₁-C₆ alkyl substituted with OH; and R₁₃ can be C₁-C₆ alkyl substituted with NH(=NH)NH(Rₑ) or N(Rₑ)₂, in which each Rₑ, independently, can be H or C₁-C₆ alkyl. Examples of such compounds include

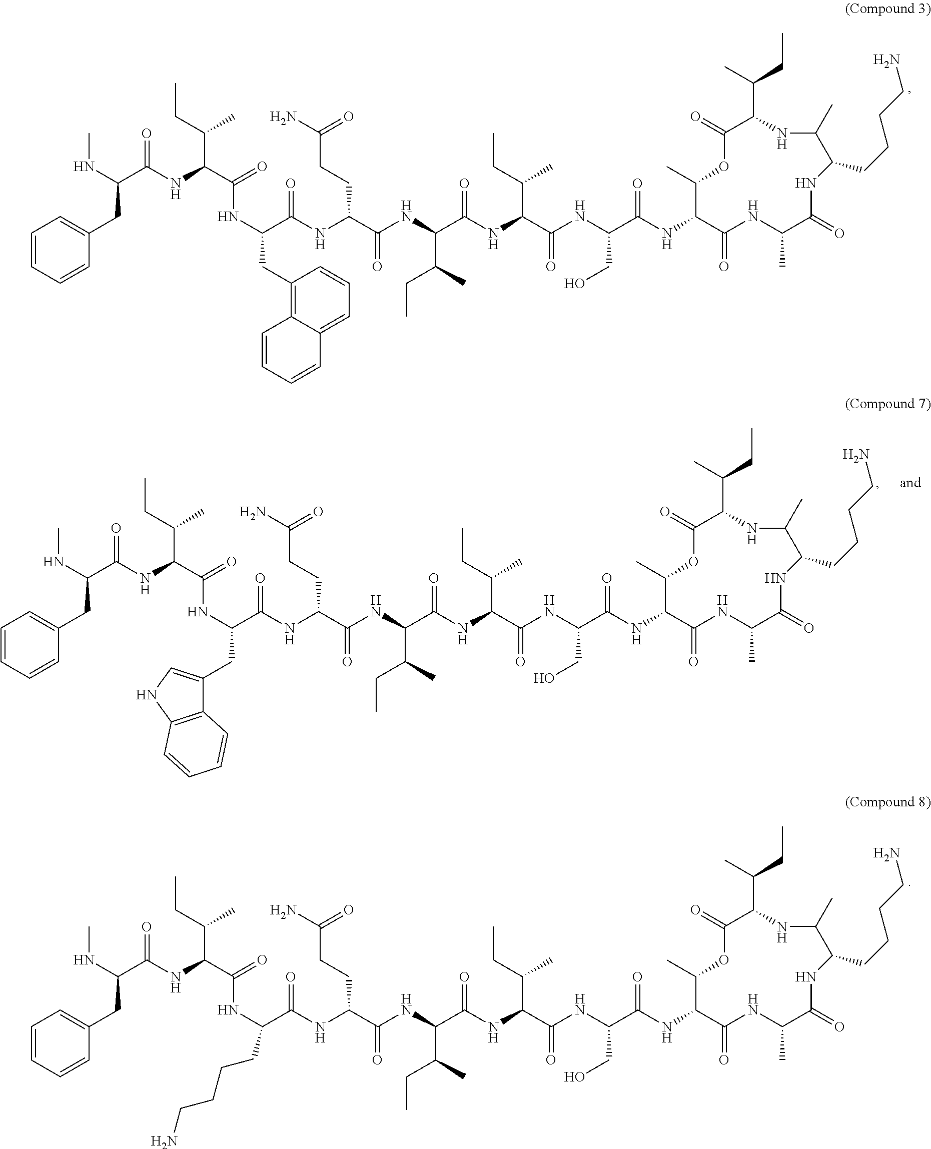

A third subset of compounds of formula (I) or (II) are those in which n is 0 or 1; each of R₁ and R₁′, independently, is H, C₁-C₆ alkyl, C(O)—Rₐ, or C(O)O—Rₐ, in which Rₐ is C₁-C₆ alkyl optionally substituted with NH₂, aryl, or heteroaryl; R₂ is C₁-C₆ alkyl optionally substituted with aryl or heteroaryl, in which the aryl or heteroaryl group is substituted with halo, NH₂, C₁-C₆ alkyl, or C₁-C₆ alkoxy; R₃ is H, C₁-C₆ alkyl, or C(O)—R_{b}, in which R_{b} is C₁-C₆ alkyl; each of R₄ and R₄′, independently, is H, C₁-C₆ alkyl, aryl, or heteroaryl; R₅ is C₁-C₆ alkyl optionally substituted with OH, NH—R_{c}, aryl, or heteroaryl, or aryl optionally substituted with C₁-C₆ alkyl, in which R_{c} is H, C(O)O—R_{c}′, or —SO₂-phenyl optionally substituted with C₁-C₆ alkyl, R_{c}′ being C₁-C₆ alkyl or C₁-C₆ alkenyl; R₆ is C₁-C₆ alkyl optionally substituted with C(O)—NH(R_{d}), NH(R_{d}), NHC(O)—R_{d}, aryl, or heteroaryl, in which each R_{d}, independently, is H, aryl, heteroaryl, or C₁-C₆ alkyl optionally substituted with aryl; R₇ is H, C₁-C₆ alkyl, aryl, or heteroaryl; R₈ is H, C₁-C₆ alkyl, aryl, or heteroaryl; R₉ is H, C₁-C₆ alkyl, aryl, or heteroaryl; R₁₀ is H, C₁-C₆ alkyl, aryl, or heteroaryl; RH is C₁-C₆ alkyl optionally substituted with OH, NH₂, aryl, or heteroaryl; R₁₂ is H or C₁-C₆ alkyl; R₁₃ is C₁-C₆ alkyl optionally substituted with aryl, heteroaryl, NH(=NH)NH(Rₑ), NH(=O)NH(Rₑ), N(Rₑ)₂, N(Rₑ)₃⁺, COORₑ, or CO—NH(CH₂)₂N(Rₑ)₂, in which each Rₑ, independently, is H, NO₂, C₁-C₆ alkyl, or C(O)—Rₑ′, Rₑ′ being C₁-C₆ alkyl; and R₁₄ is C₁-C₆ alkyl optionally substituted with NH₂.

In such embodiments, n can be 0; each of R₁ and R₁′ can be H; R₂ can be C₁-C₆ alkyl substituted with phenyl, in which phenyl can be substituted with halo; R₃ can be H; R₄ can be C₁-C₆ alkyl and R₄′ can be C₁-C₆ alkyl; R₅ can be C₁-C₆ alkyl substituted with OH; R₆ can be C₁-C₆ alkyl substituted with C(O)NH₂; each of R₇, R₈, R₉, R₁₀, R₁₂, and R₁₄ can be C₁-C₆ alkyl; R₁₁ can be C₁-C₆ alkyl substituted with OH; and R₁₃ can be C₁-C₆ alkyl substituted with NH₂. An example of such compounds is

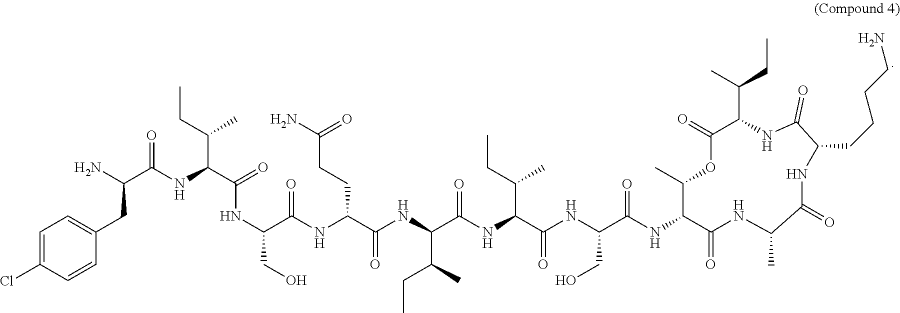 [SMILES: CC[C@H](C)[C@H](NC(=O)[C@H](N)Cc1ccc(Cl)cc1)C(=O)N[C@@H](CO)C(=O)N[C@H](CCC(N)=O)C(=O)N[C@@H](C(=O)N[C@H](C(=O)N[C@@H](CO)C(=O)N[C@H]1C(=O)N[C@@H](C)C(=O)N[C@@H](CCCCN)C(=O)N[C@@H]([C@@H](C)CC)C(=O)O[C@H]1C)[C@@H](C)CC)[C@@H](C)CC]

A fourth subset of compounds of formula (I) or (II) are those in which n is 0 or 1; each of R₁ and R₁′, independently, is H, C₁-C₆ alkyl, C(O)—Rₐ, or C(O)O—Rₐ, in which Rₐ is C₁-C₆ alkyl optionally substituted with NH₂, aryl, or heteroaryl; R2 is C₁-C₆ alkyl optionally substituted with aryl or heteroaryl, in which the aryl or heteroaryl group is optionally substituted with halo, NH₂, C₁-C₆ alkyl, or C₁-C₆ alkoxy; R₃ is H, C₁-C₆ alkyl, or C(O)—R_{b}, in which R_{b} is C₁-C₆ alkyl; each of R₄ and R₄′, independently, is H, C₁-C₆ alkyl, aryl, or heteroaryl; R₅ is C₁-C₆ alkyl optionally substituted with OH, NH—R_{c}, aryl, or heteroaryl, or aryl optionally substituted with C₁-C₆ alkyl, in which R_{c} is H, C(O)O—R_{c}′, or —SO₂-phenyl optionally substituted with C₁-C₆ alkyl, R_{c}′ being C₁-C₆ alkyl or C₁-C₆ alkenyl; R₆ is C₁-C₆ alkyl optionally substituted with C(O)—NH(R_{d}), NH(R_{d}), NHC(O)—R_{d}, aryl, or heteroaryl, in which each R_{d}, independently, is H, aryl, heteroaryl, or C₁-C₆ alkyl optionally substituted with aryl; R₇ is H, C₁-C₆ alkyl, aryl, or heteroaryl; R₈ is H, C₁-C₆ alkyl, aryl, or heteroaryl; R₉ is H, C₁-C₆ alkyl, aryl, or heteroaryl; R₁₀ is H, C₁-C₆ alkyl, aryl, or heteroaryl; R₁₁ is C₁-C₆ alkyl optionally substituted with OH, NH₂, aryl, or heteroaryl; R₁₂ is H or C₁-C₆ alkyl; R₁₃ is C₁, C₂, C₅ or C₆ alkyl optionally substituted with aryl, heteroaryl, NH(=NH)NH(Rₑ), NH(=O)NH(Rₑ), N(Rₑ)₂, N(Rₑ)₂, COORₑ, or CO—NH(CH₂)₂N(Rₑ)₂, in which each Rₑ, independently, is H, NO₂, C₁-C₆ alkyl, or C(O)—Rₑ′, Rₑ′ being C₁-C₆ alkyl; and R₁₄ is C₁-C₆ alkyl optionally substituted with NH₂.

In such embodiments, n can be 0; R₁ can be C₁-C₆ alkyl; R₁′ can be H; R₂ can be C₁-C₆ alkyl substituted with phenyl; R3 can be H; R4 can be C₁-C₆ alkyl; R₄′ can be C₁-C₆ alkyl; R5 can be C₁-C₆ alkyl substituted with OH; R₆ can be C₁-C₆ alkyl substituted with C(0)NH₂; each of R₇, R₈, R₉, R₁₀, R₁₂, and R₁₄ can be C₁-C₆ alkyl; R₁₁ can be C₁-C₆ alkyl substituted with OH; and R₁₃ can be C₁, C₂, C₅ or C₆ alkyl substituted with NH₂. An example of such compounds is

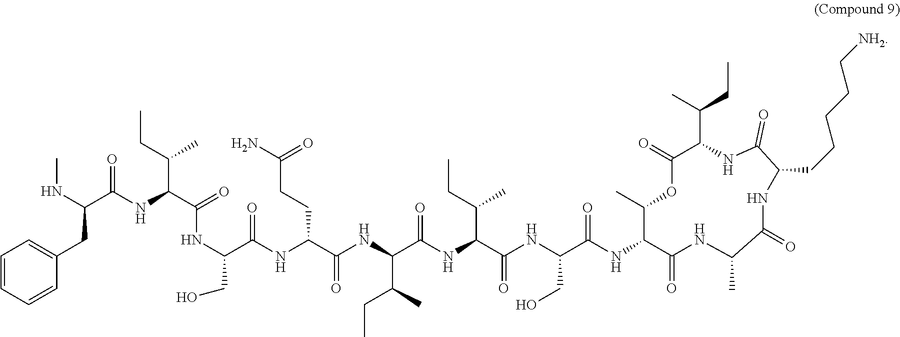 [SMILES: CC[C@H](C)[C@H](NC(=O)[C@@H](Cc1ccccc1)NC)C(=O)N[C@@H](CO)C(=O)N[C@H](CCC(N)=O)C(=O)N[C@@H](C(=O)N[C@H](C(=O)N[C@@H](CO)C(=O)N[C@H]1C(=O)N[C@@H](C)C(=O)N[C@@H](CCCCCN)C(=O)N[C@@H]([C@@H](C)CC)C(=O)O[C@H]1C)[C@@H](C)CC)[C@@H](C)CC]

A fifth subset of compounds of formula (I) or (II) are those in which n is 0 or 1; each of R₁ and R₁′, independently, is H, C₁-C₆ alkyl, C(O)—Rₐ, or C(O)O—Rₐ, in which Rₐ is C₁-C₆ alkyl optionally substituted with NH₂, aryl, or heteroaryl; R₂ is C₁-C₆ alkyl optionally substituted with aryl or heteroaryl, in which the aryl or heteroaryl group is optionally substituted with halo, NH₂, C₁-C₆ alkyl, or C₁-C₆ alkoxy; R₃ is H, C₁-C₆ alkyl, or C(O)—R_{b}, in which R_{b} is C₁-C₆ alkyl; R₄ is H, C₁-C₆ alkyl, aryl, or heteroaryl; R₄′ is H, C₃-C₆ alkyl, aryl, or heteroaryl; R₅ is C₁-C₆ alkyl optionally substituted with OH, NH—R_{c}, aryl, or heteroaryl, or aryl optionally substituted with C₁-C₆ alkyl, in which R_{c} is H, C(O)O—R_{c}′ or —SO₂-phenyl optionally substituted with C₁-C₆ alkyl, R_{c}′ being C₁-C₆ alkyl or C₁-C₆ alkenyl; R₆ is C₁-C₆ alkyl optionally substituted with C(O)-NH(R_{d}), NH(R_{d}), NHC(O)-R_{d}, aryl, or heteroaryl, in which each R_{d}, independently, is H, aryl, heteroaryl, or C₁-C₆ alkyl optionally substituted with aryl; R₇ is H, C₁-C₆ alkyl, aryl, or heteroaryl; R₈ is H, C₁-C₆ alkyl, aryl, or heteroaryl; R₉ is H, C₁-C₆ alkyl, aryl, or heteroaryl; R₁₀ is H, C₁-C₆ alkyl, aryl, or heteroaryl; R₁₁ is C₁-C₆ alkyl optionally substituted with OH, NH₂, aryl, or heteroaryl; R₁₂ is H or C₁-C₆ alkyl; R₁₃ is C₁-C₆ alkyl optionally substituted with aryl, heteroaryl, NH(=NH)NH(Rₑ), NH(=O)NH(Rₑ), N(Rₑ)₂, N(Rₑ)₃⁺, COORe, or CO—NH(CH₂)₂N(Rₑ)₂, in which each Rₑ, independently, is H, NO₂, C₁-C₆ alkyl, or C(O)—Rₑ′, Rₑ′ being C₁-C₆ alkyl; and R₁₄ is C₁-C₆ alkyl optionally substituted with NH₂.

In such embodiments, n can be 0; R₁ can be C₁-C₆ alkyl; R₁′ can be H; R₂ can be C₁-C₆ alkyl substituted with phenyl; R₃ can be H; R₄ can be C₁-C₆ alkyl and R₄′ can be H; R₅ can be C₁-C6 alkyl substituted with OH; R₆ can be C₁-C6 alkyl substituted with C(O)NH₂; each of R₇, R₈, R₉, R₁₀, R₁₂, and R₁₄ can be C₁-C₆ alkyl; R₁₁ can be C₁-C₆ alkyl substituted with OH; and R₁₃ can be C₁-C₆ alkyl substituted with NH₂. An example of such compounds is

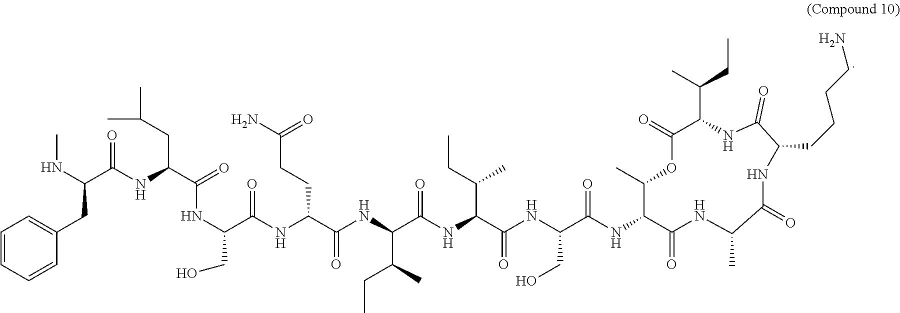 [SMILES: CC[C@H](C)[C@H](NC(=O)[C@H](NC(=O)[C@@H](CCC(N)=O)NC(=O)[C@H](CO)NC(=O)[C@H](CC(C)C)NC(=O)[C@@H](Cc1ccccc1)NC)[C@@H](C)CC)C(=O)N[C@@H](CO)C(=O)N[C@H]1C(=O)N[C@@H](C)C(=O)N[C@@H](CCCCN)C(=O)N[C@@H]([C@@H](C)CC)C(=O)O[C@H]1C]

In some embodiments, a subset of the antimicrobial peptides of formula (I) or (II) are those of formula (III) or a pharmaceutically acceptable salt thereof:

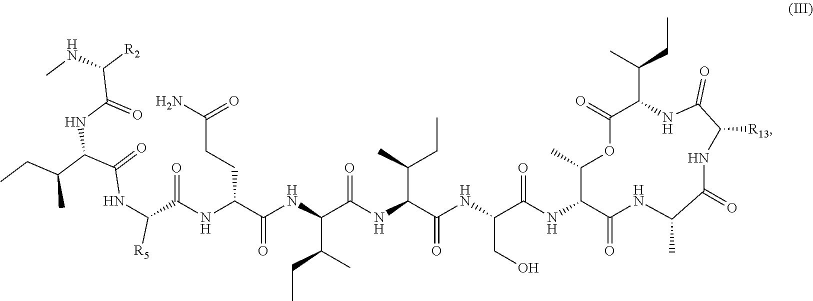

in which R₂ is C₁-C₆ alkyl optionally substituted with aryl or heteroaryl, in which the aryl or heteroaryl group is optionally substituted with halo, NH₂, C₁-C₆ alkyl, or C₁-C₆ alkoxy; R₅ is C₁-C₆ alkyl optionally substituted with OH, NH—R_{c}, aryl, or heteroaryl, or aryl optionally substituted with C₁-C₆ alkyl, in which Rₐ is H, C(O)O—R_{c}′, or —SO₂-phenyl optionally substituted with C₁-C₆ alkyl, R_{c}′ being C₁-C₆ alkyl or C₁-C₆ alkenyl; and R₁₃ is C₁-C₆ alkyl optionally substituted with heteroaryl, NH(=NH)NH(Rₑ), NH(=O)NH(Rₑ), N(Rₑ)₂, N(Rₑ)₃⁺, COORₑ, COO—NH(CH₂)₂N(Rₑ)₂, or aryl optionally substituted with NH(=NH)NH(Rₑ), in which each Rₑ, independently, is H, NO₂, C₁-C₆ alkyl optionally substituted with aryl, or C(O)—Rₑ′, Rₑ′ being C₁-C₆ alkyl.

In some embodiments, R₂ in formula (III) is C₁-C₆ alkyl substituted with phenyl, chlorophenyl, methoxyphenyl, or naphthyl.

In some embodiments, R₅ in formula (III) is C₁-C₆ alkyl optionally substituted with OH, NH—R_{c}, indolyl, naphthyl, in which R_{c} is H, C(O)O-allyl, or —SO₂-tosyl.

In some embodiments, R₁₃ in formula (III) is C₁-C₆ alkyl optionally substituted with NH(=NH)NH₂, NHCH₂Ph, or phenyl substituted with NH(=NH)NH₂.

Examples of compounds of formula (III) include

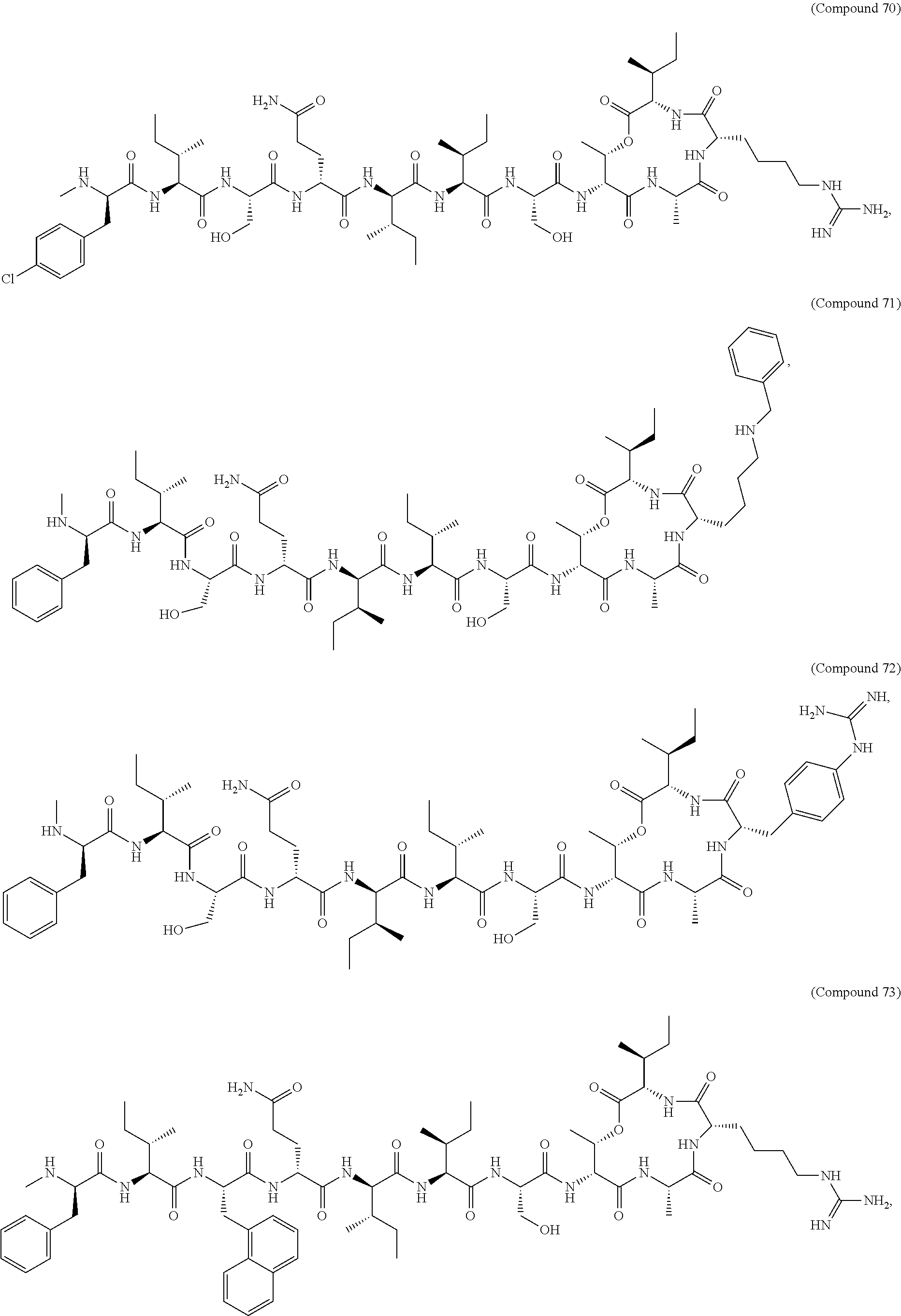

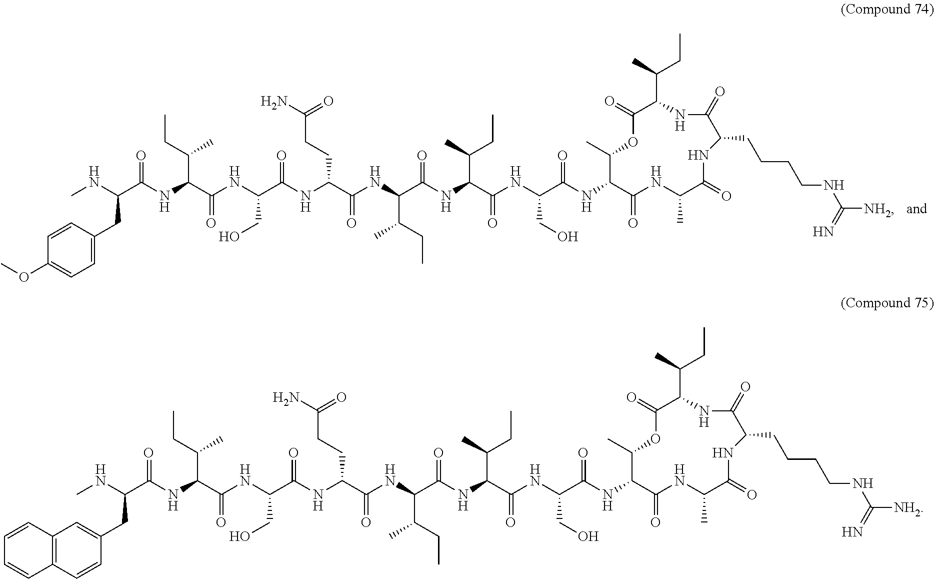 [SMILES: CC[C@H](C)[C@H](NC(=O)[C@@H](Cc1ccc(OC)cc1)NC)C(=O)N[C@@H](CO)C(=O)N[C@H](CCC(N)=O)C(=O)N[C@@H](C(=O)N[C@H](C(=O)N[C@@H](CO)C(=O)N[C@H]1C(=O)N[C@@H](C)C(=O)N[C@@H](CCCCNC(=N)N)C(=O)N[C@@H]([C@@H](C)CC)C(=O)O[C@H]1C)[C@@H](C)CC)[C@@H](C)CC ; CC[C@H](C)[C@H](NC(=O)[C@@H](Cc1ccc2ccccc2c1)NC)C(=O)N[C@@H](CO)C(=O)N[C@H](CCC(N)=O)C(=O)N[C@@H](C(=O)N[C@H](C(=O)N[C@@H](CO)C(=O)N[C@H]1C(=O)N[C@@H](C)C(=O)N[C@@H](CCCCNC(=N)N)C(=O)N[C@@H]([C@@H](C)CC)C(=O)O[C@H]1C)[C@@H](C)CC)[C@@H](C)CC]

In some embodiments, the stereochemistry of the compounds of formula (III) can be shown in the following formula:

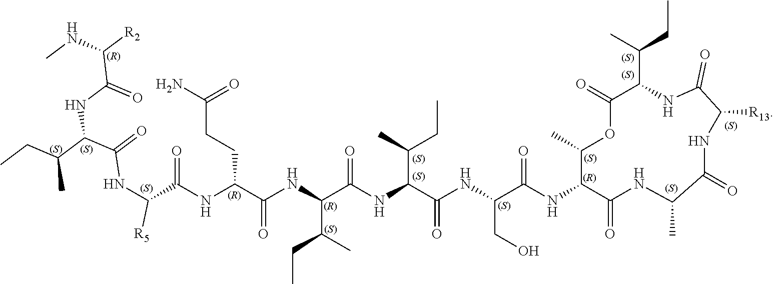

In some embodiments, each chiral center in the compounds of formula (I) or (II) has the same S or R configuration as the corresponding chiral center in the compounds of formula (III).

Exemplary compounds of formula (I) (i.e., Compounds 1-75) include those listed in Table 1 below.

TABLE 1

| Cpd # | Amino Acid Components |
|---|---|
| 1 | H-D-MePhe-Ile-Lys-D-Gln-D-aIle-Ile-Ser-c(D-Thr-Ala-Har-Ile) |
| 2 | H-D-MePhe-Ile-Trp-D-Gln-D-aIle-Ile-Ser-c(D-Thr-Ala-Har-Ile) |
| 3 | H-D-MePhe-Ile-1Nal-D-Gln-D-aIle-Ile-Ser-c(D-Thr-Ala-Lys-Ile) |
| 4 | H-D-Phe(4-Cl)-Ile-Ser-D-Gln-D-aIle-Ile-Ser-c(D-Thr-Ala-Lys-Ile) |
| 5 | H-D-MePhe-Ile-Ser-D-Gln-D-aIle-Ile-Ser-c(D-Thr-Ala-Har-Ile) |
| 6 | H-D-MePhe-Ile-Ser-D-Gln-D-aIle-Ile-Ser-c(D-Thr-Ala-Har(Me)-Ile) |
| 7 | H-D-MePhe-Ile-Trp-D-Gln-D-aIle-Ile-Ser-c(D-Thr-Ala-Lys-Ile) |
| 8 | H-D-MePhe-Ile-Lys-D-Gln-D-aIle-Ile-Ser-c(D-Thr-Ala-Lys-Ile) |
| 9 | H-D-MePhe-Ile-Ser-D-Gln-D-aIle-Ile-Ser-c(D-Thr-Ala-hLys-Ile) |
| 10 | H-D-MePhe-Leu-Ser-D-Gln-D-aIle-Ile-Ser-c(D-Thr-Ala-Lys-Ile) |
| 11 | H-D-MePhe-Ile-Ser-D-Gln-D-aIle-Ile-Ser-c(D-Thr-Ala-Arg(Me)-Ile) |
| 12 | H-D-MePhe-Ile-Ser-D-Gln-D-Ile-Ile-Ser-c(D-Thr-Ala-Lys-Ile) |
| 13 | H-D-MePhe-Ile-Ser-D-Trp-D-aIle-Ile-Ser-c(D-Thr-Ala-Lys-Ile) |
| 14 | H-D-MePhe-Ile-Ser-D-Lys-D-aIle-Ile-Ser-c(D-Thr-Ala-Lys-Ile) |
| 15 | H-D-MePhe-Ile-Ser-D-Gln-D-Leu-Ile-Ser-c(D-Thr-Ala-Lys-Ile) |
| 16 | H-D-MePhe-Ile-Lys(Tos)-D-Gln-D-aIle-Ile-Thr-c(D-Thr-Ala-Lys-Ile) |
| 17 | H-D-MePhe-Ile-Ala-D-Gln-D-aIle-Ile-Ser-c(D-Thr-Ala-Lys-Ile) |
| 18 | H-D-MePhe-Ile-Ile-D-Gln-D-aIle-Ile-Ser-c(D-Thr-Ala-Lys-Ile) |
| 19 | H-D-MePhe-Ile-Ser-D-Leu-D-aIle-Ile-Ser-c(D-Thr-Ala-Lys-Ile) |
| 20 | H-D-MePhe-Ile-Ser-D-Gln-D-aIle-Ile-Ser-c(D-Thr-Ala-Dab-Ile) |
| 21 | H-D-MePhe-Ile-Ser-D-Gln-D-aIle-Ile-Ser-c(D-Thr-Ala-Agb-Ile) |
| 22 | H-D-MePhe-Ile-Ser-D-Gln-D-aIle-Ile-Ser-c(D-Thr-Ala-Agb(Me)-Ile) |
| 23 | H-D-MePhe-Ile-Ser-D-Ala-D-aIle-Ile-Ser-c(D-Thr-Ala-Lys-Ile) |
| 24 | H-D-MePhe-Ile-Ser-D-Lys(Nicotinoyl)-D-aIle-Ile-Thr-c(D-Thr-Ala-Lys-Ile) |
| 25 | H-D-MePhe-Ile-Lys(Alloc)-D-Gln-D-aIle-Ile-Thr-c(D-Thr-Ala-Lys-Ile) |
| 26 | H-D-MePhe-Nle-Ser-D-Gln-D-aIle-Ile-Ser-c(D-Thr-Ala-Lys-Ile) |
| 27 | H-D-Phe-Ile-Ser-D-Gln-D-aIle-Ile-Ser-c(D-Thr-Ala-Lys-Ile) |
| 28 | H-D-MePhe-Ile-Ser-D-Gln-D-aIle-Ile-Ser-c(D-Thr-Ala-Lys(Ac)-Ile) |
| 29 | H-D-MePhe-Ile-Ser-D-Gln-D-aIle-Ile-Ser-c(D-Thr-Ala-hCit(Me)-Ile) |
| 30 | H-D-MePhe-Ile-Ser-D-Gln-D-aIle-Ile-Ser-c(D-Thr-Ala-Lys(Me₃)-Ile) |
| 31 | H-D-MePhe-Ile-Ser-D-Gln-D-aIle-Ile-Ser-c(D-Ser-Ala-Har-Ile) |
| 32 | H-D-MePhe-Ile-Ser-D-Gln-D-aIle-Ile-Thr-c(D-Thr-Ala-Lys-Ile) |
| 33 | H-D-MePhe-Ile-Ser-D-Gln-D-aIle-Nle-Ser-c(D-Thr-Ala-Lys-Ile) |
| 34 | H-D-MePhe-Ile-Ser-D-Gln-D-aIle-Ile-Ser-c(D-Thr-Ala-Glu(NH(CH₂)₂NMe₂)-Ile) |
| 35 | H-D-MePhe-Ile-Ser-D-Gln-D-aIle-Ile-Ser-c(D-Thr-Ala-Glu(NH(CH₂)₂NH₂)-Ile) |
| 36 | H-D-MePhe-Ile-Ser-D-Gln-D-aIle-Ile-Ser-c(D-Thr-Ala-Orn(iPr)-Ile) |
| 37 | H-D-MePhe-Ile-Ser-D-Gln-D-aIle-Leu-Ser-c(D-Thr-Ala-Lys-Ile) |
| 38 | H-D-MePhe-Ile-Ser-D-Gln-D-aIle-Ile-Ser-c(D-Thr-Ala-Orn(Ac)-Ile) |
| 39 | H-D-MePhe-Ile-Ser-D-Gln-D-aIle-Ile-Ser-c(D-Thr-Ala-Cit(Me)-Ile) |
| 40 | H-D-MePhe-Ile-Ser-D-Gln-D-aIle-Ile-Ser-c(D-Thr-Ala-Ala-D-aIle) |
| 41 | H-D-MePhe-Ile-Ser-D-Gln-D-aIle-Ile-Ser-c(D-Thr-Ala-His-L-Ile) |
| 42 | H-D-MePhe-Ile-Ser-D-Gln-D-aIle-Ile-Ser-c(D-Ser-Ala-Lys-Ile) |
| 43 | H-D-MePhe-Ile-Ser-D-Gln-D-aIle-Trp-Ser-c(D-Thr-Ala-Lys-Ile) |
| 44 | H-D-MePhe-Ile-Ser-D-Gln-D-aIle-Ile-Ser-c(D-Thr-Ala-Arg(NO₂)-Ile) |
| 45 | H-D-MePhe-Ile-Ser-D-Gln-D-aIle-Ile-Ser-c(D-Thr-Ala-Dab(Ac)-Ile) |
| 46 | H-D-MePhe-Ile-Ser-D-Gln-D-aIle-Ile-Ser-c(D-Thr-Ala-norCit(Me)-Ile) |
| 47 | H-D-MePhe-Trp-Ser-D-Gln-D-aIle-Ile-Ser-c(D-Thr-Ala-Lys-Ile) |
| 48 | H-D-MePhe-Ile-Ser-D-Gln-D-aIle-Ile-Ser-c(D-Thr-Ala-bhLys-Ile) |
| 49 | Fmoc-D-MePhe-Ile-Ser-D-Gln-D-aIle-Ile-Ser-c(D-Thr-Ala-Lys-Ile) |
| 50 | H-D-Leu-Ile-Ser-D-Gln-D-aIle-Ile-Ser-c(D-Thr-Ala-Lys-Ile) |
| 51 | H-D-MePhe-Ile-Ser-D-Gln-D-Trp-Ile-Ser-c(D-Thr-Ala-Lys-Ile) |
| 52 | H-D-MePhe-Ile-Ser-D-Gln-D-aIle-Ile-Ser-c(D-Thr-Ala-Ala-Ile) |
| 53 | H-D-MePhe-Ala-Ser-D-Gln-D-aIle-Ile-Ser-c(D-Thr-Ala-Lys-Ile) |
| 54 | H-D-MePhe-Ile-Ser-D-Gln-D-aIle-Ile-Ala-c(D-Thr-Ala-Lys-Ile) |
| 55 | H-D-MePhe-Ile-Ser-D-Gln-D-aIle-Ile-Hse-c(D-Thr-Ala-Lys-Ile) |
| 56 | H-D-MePhe-Ile-Ser-D-Gln-D-aIle-Ile-Ser-c(D-Thr-Ala-Glu-Ile) |
| 57 | H-D-MePhe-Ile-Ser-D-Gln-D-aIle-Ile-Ser-c(D-Thr-Ala-His-[D-aIle]) |
| 58 | H-D-MePhe-Ile-Ser-D-Gln-D-aIle-Ile-Ile-c(D-Thr-Ala-Lys-Ile) |
| 59 | H-D-Trp-Ile-Ser-D-Gln-D-aIle-Ile-Ser-c(D-Thr-Ala-Lys-Ile) |
| 60 | H-D-MePhe-Ile-Ser-D-Gln-D-aIle-Ile-Trp-c(D-Thr-Ala-Lys-Ile) |
| 61 | H-D-MePhe-Ile-Ser-D-Gln-D-Ala-Ile-Ser-c(D-Thr-Ala-Lys-Ile) |
| 62 | H-D-MePhe-Ile-Ser-D-Gln-D-aIle-Ile-Lys-c(D-Thr-Ala-Lys-Ile) |
| 63 | H-D-MeAla-Ile-Ser-D-Gln-D-aIle-Ile-Ser-c(D-Thr-Ala-Lys-Ile) |
| 64 | H-D-MePhe-Ile-Ser-D-Gln-D-aIle-D-aIle-Ser-c(D-Thr-Ala-Arg(Me)-Ile) |
| 65 | H-Ala-D-MePhe-Ile-Ser-D-Gln-D-aIle-Ile-Ser-c(D-Thr-Ala-Lys-Ile) |
| 66 | H-D-MePhe-Ile-Ser-D-Gln-D-aIle-Ala-Ser-c(D-Thr-Ala-Lys-Ile) |
| 67 | Ac-D-Phe-Ile-Ser-D-Gln-D-aIle-Ile-Ser-c(D-Thr-Ala-Lys-Ile) |
| 68 | H-D-MePhe-Ile-Ser-D-Gln-D-aIle-Ile-Ser-c(D-Thr-Ala-Lys-D,L-Lys) |
| 69 | H-D-MePhe-Ile-Ser-D-Gln-D-aIle-Ile-Ser-c(D-Thr-Ala-Ala-D,L-Lys) |
| 70 | H-D-MePhe(4-Cl)-Ile-Ser-D-Gln-D-aIle-Ile-Ser-c(D-Thr-Ala-Har-Ile) |
| 71 | H-D-MePhe-Ile-Ser-D-Gln-D-aIle-Ile-Ser-c(D-Thr-Ala-Lys(Bn)-Ile) |
| 72 | H-D-MePhe-Ile-Ser-D-Gln-D-aIle-Ile-Ser-c(D-Thr-Ala-Phe(4-guanidino)-Ile) |
| 73 | H-D-MePhe-Ile-lNal-D-Gln-D-aIle-Ile-Ser-c(D-Thr-Ala-Har-Ile) |
| 74 | H-D-MeTyr(Me)-Ile-Ser-D-Gln-D-aIle-Ile-Ser-c(D-Thr-Ala-Har-Ile) |
| 75 | H-D-Me2Nal-Ile-Ser-D-Gln-D-aIle-Ile-Ser-c(D-Thr-Ala-Har-Ile) |

Unless specified otherwise, the amino acid code in Table 1 refers to its L-isomer. In addition, if the substitution is before an amino acid code, it means that the substitution is at the α-NH₂ position. For example, MePhe refers to Phe substituted with a methyl group at the α-NH₂ position. If the substitution is after an amino acid code, it means that the substitution is on the side chain. For example, Lys(Me) refers to Lys substituted with a methyl group at 6-amino position.

Certain amino acid codes listed in Table 1 are listed below: Phe(4-Cl) refers to Phe substituted with a chloro group at the 4 position on the phenyl ring, Phe(4-guanidino) refers to Phe substituted with a guanidine group at the 4 position on the phenyl ring, Fmoc-D-MePhe refers to Phe substituted with a methyl group and a Fmoc group at t the α-NH₂ position, MeAla refers to alanine substituted with a methyl group at the α-NH₂ position, Ala-D-MePhe refers to Phe substituted with a methyl group and an alanine group at the α-NH₂ position, Ac-D-Phe refers to Phe substituted with an acetyl group at the a-NH₂ position, Ac-Ile refers to Ile substituted with an acetyl group at the α-NH₂ position, 1Nal refers to (1-naphthyl)-L-alanine, D-alle refers to D-allo-isoleucine, Hse refers to homoserine, Orn refers to L-ornithine, Har refers to homoarginine (aka Harg), Har(Me) refers to Har substituted with a methyl group at the NH₂ position in the guanidinyl group, hLys refers to homolysine, bhLys refers to beta-hLys, Lys(Ac) refers to Lys substituted with an acetyl group at the 6-amino position, Lys(tos) refers to Lys substituted with a tosyl group at the 6-amino position, Lys(Alloc) refers to Lys substituted with a COO-allyl group at the 6-amino position, Lys(nicotinoyl) refers to Lys substituted with a C(O)-3-pyridinyl group at the 6-amino position, Lys(Me₃) refers to Lys substituted with three methyl groups at the 6-amino position, Arg(Me) refers to Arg substituted with a methyl group at the NH₂ position in the guanidinyl group, Arg(NO2) refers to Arg substituted with a NO₂ group at the NH₂ position in the guanidinyl group, Dab refers to 2,4-diaminobutyric acid, Dab(Ac) refers to Dab substituted with an acetyl group at 5-amino position, Agb refers to norarginine, Agb(Me) refers to Agb substituted with a methyl group at the NH₂ position in the guanidinyl group, hCit(Me) refers to homocitrulline substituted with a methyl group at the NH₂ position in the urea group, norCit(Me) refers to norcitrulline substituted with a methyl group at the NH₂ position in the urea group, Cit(Me) refers to citrulline substituted with a methyl group at the NH₂ position in the urea group, Glu(NH(CH₂)₂NMe₂) refers to Glu substituted with NH(CH₂)₂NMe₂ at 4-carboxyl position, Glu(NH(CH₂)₂NH₂) refers to Glu substituted with NH(CH₂)₂NH₂ at 4-carboxyl position, Orn(iPr) refers to Orn substituted with an isopropyl group at the 5-amino position, and Orn(Ac) refers to Orn substituted with an acetyl group at the 5-amino position,. Other amino acid codes listed in Table 1 are well known in the art.

Exemplary Compounds 1-75 are those of formula (II), in which n, R₁, R₁′, R₂, R₃, R₄, R_{4′}, and R₅-R₁₄ are those shown in Tables 2-1 and 2-2 below.

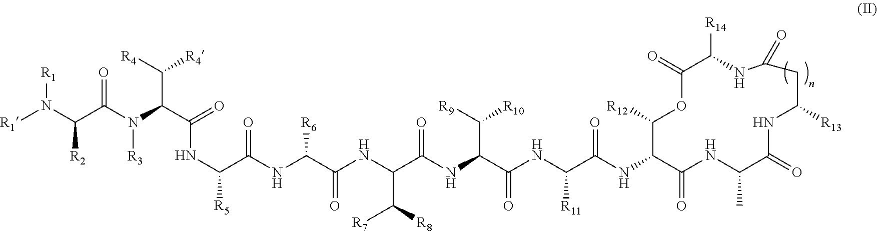

TABLE 2-1

| Cpd | | | | | | | | | |
|---|---|---|---|---|---|---|---|---|---|
| # | n | R₁ | R₁’ | R₂ | R₃ | R₄ | R₄’ | R₅ | R₆ |
| 1 | 0 | CH₃ | H | CH₂Ph | H | CH₂CH₃ | CH₃ | (CH₂)₄NH₂ | (CH₂)₂CONH₂ |
| 2 | 0 | CH₃ | H | CH₂Ph | H | CH₂CH₃ | CH₃ | CH₂(1H- | (CH₂)₂CONH₂ |
| | | | | | | | | indol-3-yl) | |
| 3 | 0 | CH₃ | H | CH₂Ph | H | CH₂CH₃ | CH₃ | CH₂(1- | (CH₂)₂CONH₂ |
| | | | | | | | | naphthyl) | |
| 4 | 0 | H | H | CH₂-(4- | H | CH₂CH₃ | CH₃ | CH₂OH | (CH₂)₂CONH₂ |
| | | | | chlorophenyl) | | | | | |
| 5 | 0 | CH₃ | H | CH₂Ph | H | CH₂CH₃ | CH₃ | CH₂OH | (CH₂)₂CONH₂ |
| 6 | 0 | CH₃ | H | CH₂Ph | H | CH₂CH₃ | CH₃ | CH₂OH | (CH₂)₂CONH₂ |
| 7 | 0 | CH₃ | H | CH₂Ph | H | CH₂CH₃ | CH₃ | CH₂(1H- | (CH₂)₂CONH₂ |
| | | | | | | | | indol-3-yl) | |
| 8 | 0 | CH₃ | H | CH₂Ph | H | CH₂CH₃ | CH₃ | (CH₂)₄NH₂ | (CH₂)₂CONH₂ |
| 9 | 0 | CH₃ | H | CH₂Ph | H | CH₂CH₃ | CH₃ | CH₂OH | (CH₂)₂CONH₂ |
| 10 | 0 | CH₃ | H | CH₂Ph | H | CH(CH₃)₂ | H | CH₂OH | (CH₂)₂CONH₂ |
| 11 | 0 | CH₃ | H | CH₂Ph | H | CH₂CH₃ | CH₃ | CH₂OH | (CH₂)₂CONH₂ |
| 12 | 0 | CH₃ | H | CH₂Ph | H | CH₂CH₃ | CH₃ | CH₂OH | (CH₂)₂CONH₂ |
| 13 | 0 | CH₃ | H | CH₂Ph | H | CH₂CH₃ | CH₃ | CH₂OH | CH₂(1H-indol- |
| | | | | | | | | | 3-yl) |
| 14 | 0 | CH₃ | H | CH₂Ph | H | CH₂CH₃ | CH₃ | CH₂OH | (CH₂)₄NH₂ |
| 15 | 0 | CH₃ | H | CH₂Ph | H | CH₂CH₃ | CH₃ | CH₂OH | (CH₂)₂CONH₂ |
| 16 | 0 | CH₃ | H | CH₂Ph | H | CH₂CH₃ | CH₃ | (CH₂)₄NH— | (CH₂)₂CONH₂ |
| | | | | | | | | SO₂-Tos | |
| 17 | 0 | CH₃ | H | CH₂Ph | H | CH₂CH₃ | CH₃ | CH₃ | (CH₂)₂CONH₂ |
| 18 | 0 | CH₃ | H | CH₂Ph | H | CH₂CH₃ | CH₃ | 2-butyl | (CH₂)₂CONH₂ |
| 19 | 0 | CH₃ | H | CH₂Ph | H | CH₂CH₃ | CH₃ | CH₂OH | CH₂CH(CH₃)₂ |
| 20 | 0 | CH₃ | H | CH₂Ph | H | CH₂CH₃ | CH₃ | CH₂OH | (CH₂)₂CONH₂ |
| 21 | 0 | CH₃ | H | CH₂Ph | H | CH₂CH₃ | CH₃ | CH₂OH | (CH₂)₂CONH₂ |
| 22 | 0 | CH₃ | H | CH₂Ph | H | CH₂CH₃ | CH₃ | CH₂OH | (CH₂)₂CONH₂ |
| 23 | 0 | CH₃ | H | CH₂Ph | H | CH₂CH₃ | CH₃ | CH₂OH | CH₃ |
| 24 | 0 | CH₃ | H | CH₂Ph | H | CH₂CH₃ | CH₃ | CH₂OH | (CH₂)₄NH- |
| | | | | | | | | | C(O)-3- |
| | | | | | | | | | pyridinyl |
| 25 | 0 | CH₃ | H | CH₂Ph | H | CH₂CH₃ | CH₃ | (CH₂)₄NH— | (CH₂)₂CONH₂ |
| | | | | | | | | CO₂-allyl | |
| 26 | 0 | CH₃ | H | CH₂Ph | H | (CH₂)₂CH₃ | H | CH₂OH | (CH₂)₂CONH₂ |
| 27 | 0 | H | H | CH₂Ph | H | CH₂CH₃ | CH₃ | CH₂OH | (CH₂)₂CONH₂ |
| 28 | 0 | CH₃ | H | CH₂Ph | H | CH₂CH₃ | CH₃ | CH₂OH | (CH₂)₂CONH₂ |
| 29 | 0 | CH₃ | H | CH₂Ph | H | CH₂CH₃ | CH₃ | CH₂OH | (CH₂)₂CONH₂ |
| 30 | 0 | CH₃ | H | CH₂Ph | H | CH₂CH₃ | CH₃ | CH₂OH | (CH₂)₂CONH₂ |
| 31 | 0 | CH₃ | H | CH₂Ph | H | CH₂CH₃ | CH₃ | CH₂OH | (CH₂)₂CONH₂ |
| 32 | 0 | CH₃ | H | CH₂Ph | H | CH₂CH₃ | CH₃ | CH₂OH | (CH₂)₂CONH₂ |
| 33 | 0 | CH₃ | H | CH₂Ph | H | CH₂CH₃ | CH₃ | CH₂OH | (CH₂)₂CONH₂ |
| 34 | 0 | CH₃ | H | CH₂Ph | H | CH₂CH₃ | CH₃ | CH₂OH | (CH₂)₂CONH₂ |
| 35 | 0 | CH₃ | H | CH₂Ph | H | CH₂CH₃ | CH₃ | CH₂OH | (CH₂)₂CONH₂ |
| 36 | 0 | CH₃ | H | CH₂Ph | H | CH₂CH₃ | CH₃ | CH₂OH | (CH₂)₂CONH₂ |
| 37 | 0 | CH₃ | H | CH₂Ph | H | CH₂CH₃ | CH₃ | CH₂OH | (CH₂)₂CONH₂ |
| 38 | 0 | CH₃ | H | CH₂Ph | H | CH₂CH₃ | CH₃ | CH₂OH | (CH₂)₂CONH₂ |
| 39 | 0 | CH₃ | H | CH₂Ph | H | CH₂CH₃ | CH₃ | CH₂OH | (CH₂)₂CONH₂ |
| 40 | 0 | CH₃ | H | CH₂Ph | H | CH₂CH₃ | CH₃ | CH₂OH | (CH₂)₂CONH₂ |
| 41 | 0 | CH₃ | H | CH₂Ph | H | CH₂CH₃ | CH₃ | CH₂OH | (CH₂)₂CONH₂ |
| 42 | 0 | CH₃ | H | CH₂Ph | H | CH₂CH₃ | CH₃ | CH₂OH | (CH₂)₂CONH₂ |
| 43 | 0 | CH₃ | H | CH₂Ph | H | CH₂CH₃ | CH₃ | CH₂OH | (CH₂)₂CONH₂ |
| 44 | 0 | CH₃ | H | CH₂Ph | H | CH₂CH₃ | CH₃ | CH₂OH | (CH₂)₂CONH₂ |
| 45 | 0 | CH₃ | H | CH₂Ph | H | CH₂CH₃ | CH₃ | CH₂OH | (CH₂)₂CONH₂ |
| 46 | 0 | CH₃ | H | CH₂Ph | H | CH₂CH₃ | CH₃ | CH₂OH | (CH₂)₂CONH₂ |
| 47 | 0 | CH₃ | H | CH₂Ph | H | 1H-indol- | H | CH₂OH | (CH₂)₂CONH₂ |
| | | | | | | 3-yl | | | |
| 48 | 1 | CH₃ | H | CH₂Ph | H | CH₂CH₃ | CH₃ | CH₂OH | (CH₂)₂CONH₂ |
| 49 | 0 | Fmoc | CH₃ | CH₂Ph | H | CH₂CH₃ | CH₃ | CH₂OH | (CH₂)₂CONH₂ |
| 50 | 0 | H | H | CH₂CH(CH₃)₂ | H | CH₂CH₃ | CH₃ | CH₂OH | (CH₂)₂CONH₂ |
| 51 | 0 | CH₃ | H | CH₂Ph | H | CH₂CH₃ | CH₃ | CH₂OH | (CH₂)₂CONH₂ |
| 52 | 0 | CH₃ | H | CH₂Ph | H | CH₂CH₃ | CH₃ | CH₂OH | (CH₂)₂CONH₂ |
| 53 | 0 | CH₃ | H | CH₂Ph | H | H | H | CH₂OH | (CH₂)₂CONH₂ |
| 54 | 0 | CH₃ | H | CH₂Ph | H | CH₂CH₃ | CH₃ | CH₂OH | (CH₂)₂CONH₂ |
| 55 | 0 | CH₃ | H | CH₂Ph | H | CH₂CH₃ | CH₃ | CH₂OH | (CH₂)₂CONH₂ |
| 56 | 0 | CH₃ | H | CH₂Ph | H | CH₂CH₃ | CH₃ | CH₂OH | (CH₂)₂CONH₂ |
| 57 | 0 | CH₃ | H | CH₂Ph | H | CH₂CH₃ | CH₃ | CH₂OH | (CH₂)₂CONH₂ |
| 58 | 0 | CH₃ | H | CH₂Ph | H | CH₂CH₃ | CH₃ | CH₂OH | (CH₂)₂CONH₂ |
| 59 | 0 | H | H | CH₂(1H- | H | CH₂CH₃ | CH₃ | CH₂OH | (CH₂)₂CONH₂ |
| | | | | indo1-3-yl) | | | | | |
| 60 | 0 | CH₃ | H | CH₂Ph | H | CH₂CH₃ | CH₃ | CH₂OH | (CH₂)₂CONH₂ |
| 61 | 0 | CH₃ | H | CH₂Ph | H | CH₂CH₃ | CH₃ | CH₂OH | (CH₂)₂CONH₂ |
| 62 | 0 | CH₃ | H | CH₂Ph | H | CH₂CH₃ | CH₃ | CH₂OH | (CH₂)₂CONH₂ |
| 63 | 0 | CH₃ | H | CH₃ | H | CH₂CH₃ | CH₃ | CH₂OH | (CH₂)₂CONH₂ |
| 64 | 0 | CH₃ | H | CH₂Ph | H | CH₂CH₃ | CH₃ | CH₂OH | (CH₂)₂CONH₂ |
| 65 | 0 | C(O)CH- | CH₃ | CH₂Ph | H | CH₂CH₃ | CH₃ | CH₂OH | (CH₂)₂CONH₂ |
| | | (CH₃)NH₂ | | | | | | | |
| 66 | 0 | CH₃ | H | CH₂Ph | H | CH₂CH₃ | CH₃ | CH₂OH | (CH₂)₂CONH₂ |
| 67 | 0 | C(O)CH₃ | H | CH₂Ph | H | CH₂CH₃ | CH₃ | CH₂OH | (CH₂)₂CONH₂ |
| 68 | 0 | CH₃ | H | CH₂Ph | H | CH₂CH₃ | CH₃ | CH₂OH | (CH₂)₂CONH₂ |
| 69 | 0 | CH₃ | H | CH₂Ph | H | CH₂CH₃ | CH₃ | CH₂OH | (CH₂)₂CONH₂ |
| 70 | 0 | CH₃ | H | CH₂Ph(4-Cl) | H | CH₂CH₃ | CH₃ | CH₂OH | (CH₂)₂CONH₂ |
| 71 | 0 | CH₃ | H | CH₂Ph | H | CH₂CH₃ | CH₃ | CH₂OH | (CH₂)₂CONH₂ |
| 72 | 0 | CH₃ | H | CH₂Ph | H | CH₂CH₃ | CH₃ | CH₂OH | (CH₂)₂CONH₂ |
| 73 | 0 | CH₃ | H | CH₂Ph | H | CH₂CH₃ | CH₃ | CH₂(1- | (CH₂)₂CONH₂ |
| | | | | | | | | naphthyl) | |
| 74 | 0 | CH₃ | H | CH₂Ph(4- | H | CH₂CH₃ | CH₃ | CH₂OH | (CH₂)₂CONH₂ |
| | | | | OCH₃) | | | | | |
| 75 | 0 | CH₃ | H | CH₂(2- | H | CH₂CH₃ | CH₃ | CH₂OH | (CH₂)₂CONH₂ |
| | | | | naphthyl) | | | | | |

TABLE 2-2

| Cpd | | | | | | | | |
|---|---|---|---|---|---|---|---|---|
| # | R₇ | R₈ | R₉ | R₁₀ | R₁₁ | R₁₂ | R₁₃ | R₁₄ |
| 1 | CH₂CH₃ | CH₃ | CH₂CH₃ | CH₃ | CH₂OH | CH₃ | (CH₂)₄NH(=NH)NH₂ | (S)-2-butyl |
| 2 | CH₂CH₃ | CH₃ | CH₂CH₃ | CH₃ | CH₂OH | CH₃ | (CH₂)₄NH(=NH)NH₂ | (S)-2-butyl |
| 3 | CH₂CH₃ | CH₃ | CH₂CH₃ | CH₃ | CH₂OH | CH₃ | (CH₂)₄NH₂ | (S)-2-butyl |
| 4 | CH₂CH₃ | CH₃ | CH₂CH₃ | CH₃ | CH₂OH | CH₃ | (CH₂)₄NH₂ | (S)-2-butyl |
| 5 | CH₂CH₃ | CH₃ | CH₂CH₃ | CH₃ | CH₂OH | CH₃ | (CH₂)₄NH(=NH)NH₂ | (S)-2-butyl |
| 6 | CH₂CH₃ | CH₃ | CH₂CH₃ | CH₃ | CH₂OH | CH₃ | (CH₂)₄NH(=NH)NHMe | (S)-2-butyl |
| 7 | CH₂CH₃ | CH₃ | CH₂CH₃ | CH₃ | CH₂OH | CH₃ | (CH₂)₄NH₂ | (S)-2-butyl |
| 8 | CH₂CH₃ | CH₃ | CH₂CH₃ | CH₃ | CH₂OH | CH₃ | (CH₂)₄NH₂ | (S)-2-butyl |
| 9 | CH₂CH₃ | CH₃ | CH₂CH₃ | CH₃ | CH₂OH | CH₃ | (CH₂)₅NH₂ | (S)-2-butyl |
| 10 | CH₂CH₃ | CH₃ | CH₂CH₃ | CH₃ | CH₂OH | CH₃ | (CH₂)₄NH₂ | (S)-2-butyl |
| 11 | CH₂CH₃ | CH₃ | CH₂CH₃ | CH₃ | CH₂OH | CH₃ | (CH₂)3NH(=NH)NHMe | (S)-2-butyl |
| 12 | CH₃ | CH₂CH₃ | CH₂CH₃ | CH₃ | CH₂OH | CH₃ | (CH₂)₄NH₂ | (S)-2-butyl |
| 13 | CH₂CH₃ | CH₃ | CH₂CH₃ | CH₃ | CH₂OH | CH₃ | (CH₂)₄NH₂ | (S)-2-butyl |
| 14 | CH₂CH₃ | CH₃ | CH₂CH₃ | CH₃ | CH₂OH | CH₃ | (CH₂)₄NH₂ | (S)-2-butyl |
| 15 | CH(CH₃)₂ | H | CH₂CH₃ | CH₃ | CH₂OH | CH₃ | (CH₂)₄NH₂ | (S)-2-butyl |
| 16 | CH₂CH₃ | CH₃ | CH₂CH₃ | CH₃ | CH₂OH | CH₃ | (CH₂)₄NH₂ | (S)-2-butyl |
| 17 | CH₂CH₃ | CH₃ | CH₂CH₃ | CH₃ | CH₂OH | CH₃ | (CH₂)₄NH₂ | (S)-2-butyl |
| 18 | CH₂CH₃ | CH₃ | CH₂CH₃ | CH₃ | CH₂OH | CH₃ | (CH₂)₄NH₂ | (S)-2-butyl |
| 19 | CH₂CH₃ | CH₃ | CH₂CH₃ | CH₃ | CH₂OH | CH₃ | (CH₂)₄NH₂ | (S)-2-butyl |
| 20 | CH₂CH₃ | CH₃ | CH₂CH₃ | CH₃ | CH₂OH | CH₃ | (CH₂)₂NH₂ | (S)-2-butyl |
| 21 | CH₂CH₃ | CH₃ | CH₂CH₃ | CH₃ | CH₂OH | CH₃ | (CH₂)₂NH(=NH)NH₂ | (S)-2-butyl |
| 22 | CH₂CH₃ | CH₃ | CH₂CH₃ | CH₃ | CH₂OH | CH₃ | (CH₂)₄NH(=NH)NHMe | (S)-2-butyl |
| 23 | CH₂CH₃ | CH₃ | CH₂CH₃ | CH₃ | CH₂OH | CH₃ | (CH₂)₄NH₂ | (S)-2-butyl |
| 24 | CH₂CH₃ | CH₃ | CH₂CH₃ | CH₃ | CH₂OH | CH₃ | (CH₂)₄NH₂ | (S)-2-butyl |
| 25 | CH₂CH₃ | CH₃ | CH₂CH₃ | CH₃ | CH₂OH | CH₃ | (CH₂)₄NH₂ | (S)-2-butyl |
| 26 | CH₂CH₃ | CH₃ | CH₂CH₃ | CH₃ | CH₂OH | CH₃ | (CH₂)₄NH₂ | (S)-2-butyl |
| 27 | CH₂CH₃ | CH₃ | CH₂CH₃ | CH₃ | CH₂OH | CH₃ | (CH₂)₄NH₂ | (S)-2-butyl |
| 28 | CH₂CH₃ | CH₃ | CH₂CH₃ | CH₃ | CH₂OH | CH₃ | (CH₂)₄NHC(O)Me | (S)-2-butyl |
| 29 | CH₂CH₃ | CH₃ | CH₂CH₃ | CH₃ | CH₂OH | CH₃ | (CH₂)₄NHC(O)NHMe | (S)-2-butyl |
| 30 | CH₂CH₃ | CH₃ | CH₂CH₃ | CH₃ | CH₂OH | CH₃ | (CH₂)₄N(Me)₃⁺ | (S)-2-butyl |
| 31 | CH₂CH₃ | CH₃ | CH₂CH₃ | CH₃ | CH₂OH | H | (CH₂)₄NH(NH)NH₂ | (S)-2-butyl |
| 32 | CH₂CH₃ | CH₃ | CH₂CH₃ | CH₃ | CH(OH)—CH₃ | CH₃ | (CH₂)₄NH₂ | (S)-2-butyl |
| 33 | CH₂CH₃ | CH₃ | (CH₂)₂CH₃ | H | CH₂OH | CH₃ | (CH₂)₄NH₂ | (S)-2-butyl |
| 34 | CH₂CH₃ | CH₃ | CH₂CH₃ | CH₃ | CH₂OH | CH₃ | (CH₂)₂COONH— | (S)-2-butyl, |
| | | | | | | | (CH₂)₂NMe₂ | |
| 35 | CH₂CH₃ | CH₃ | CH₂CH₃ | CH₃ | CH₂OH | CH₃ | (CH₂)₂COONH— | (S)-2-butyl |
| | | | | | | | (CH₂)₂NH₂ | |
| 36 | CH₂CH₃ | CH₃ | CH₂CH₃ | CH₃ | CH₂OH | CH₃ | (CH₂)₃NH(i-Pr) | (S)-2-butyl |
| 37 | CH₂CH₃ | CH₃ | CH(CH₃)₂ | H | CH₂OH | CH₃ | (CH₂)₄NH₂ | (S)-2-butyl |
| 38 | CH₂CH₃ | CH₃ | CH₂CH₃ | CH₃ | CH₂OH | CH₃ | (CH₂)₃NHC(O)Me | (S)-2-butyl |
| 39 | CH₂CH₃ | CH₃ | CH₂CH₃ | CH₃ | CH₂OH | CH₃ | (CH₂)₂NHC(O)Me | (S)-2-butyl |
| 40 | CH₂CH₃ | CH₃ | CH₂CH₃ | CH₃ | CH₂OH | CH₃ | CH₃ | (S)-2-butyl |
| 41 | CH₂CH₃ | CH₃ | CH₂CH₃ | CH₃ | CH₂OH | CH₃ | CH₂-(1H-imidazol-4-yl) | (S)-2-butyl |
| 42 | CH₂CH₃ | CH₃ | CH₂CH₃ | CH₃ | CH₂OH | H | (CH₂)₄NH₂ | (S)-2-butyl |
| 43 | CH₂CH₃ | CH₃ | 1H-indol-3-yl | H | CH₂OH | CH₃ | (CH₂)₄NH₂ | (S)-2-butyl |
| 44 | CH₂CH₃ | CH₃ | CH₂CH₃ | CH₃ | CH₂OH | CH₃ | (CH₂)₃NH(=NH)— | (S)-2-butyl |
| | | | | | | | NHNO₂ | |
| 45 | CH₂CH₃ | CH₃ | CH₂CH₃ | CH₃ | CH₂OH | CH₃ | (CH₂)₂NHC(O)Me | (S)-2-butyl |
| 46 | CH₂CH₃ | CH₃ | CH₂CH₃ | CH₃ | CH₂OH | CH₃ | (CH₂)₂NHC(O)NHMe | (S)-2-butyl |
| 47 | CH₂CH₃ | CH₃ | CH₂CH₃ | CH₃ | CH₂OH | CH₃ | (CH₂)₄NH₂ | (S)-2-butyl |
| 48 | CH₂CH₃ | CH₃ | CH₂CH₃ | CH₃ | CH₂OH | CH₃ | (CH₂)₄NH₂ | (S)-2-butyl |
| 49 | CH₂CH₃ | CH₃ | CH₂CH₃ | CH₃ | CH₂OH | CH₃ | (CH₂)₄NH₂ | (S)-2-butyl |
| 50 | CH₂CH₃ | CH₃ | CH₂CH₃ | CH₃ | CH₂OH | CH₃ | (CH₂)₄NH₂ | (S)-2-butyl |
| 51 | 1H-indol-3-yl | H | CH₂CH₃ | CH₃ | CH₂OH | CH₃ | (CH₂)₄NH₂ | (S)-2-butyl |
| 52 | CH₂CH₃ | CH₃ | CH₂CH₃ | CH₃ | CH₂OH | CH₃ | CH₃ | (R)-2-butyl |
| 53 | CH₂CH₃ | CH₃ | CH₂CH₃ | CH₃ | CH₂OH | CH₃ | (CH₂)₄NH₂ | (S)-2-butyl |
| 54 | CH₂CH₃ | CH₃ | CH₂CH₃ | CH₃ | CH₃ | CH₃ | (CH₂)₄NH₂ | (S)-2-butyl |
| 55 | CH₂CH₃ | CH₃ | CH₂CH₃ | CH₃ | (CH₂)₂OH | CH₃ | (CH₂)₄NH₂ | (S)-2-butyl |
| 56 | CH₂CH₃ | CH₃ | CH₂CH₃ | CH₃ | CH₂OH | CH₃ | (CH₂)₂COOH | (S)-2-butyl |
| 57 | CH₂CH₃ | CH₃ | CH₂CH₃ | CH₃ | CH₂OH | CH₃ | CH₂-(1H-imidazol-4-yl) | (R)-2-butyl |
| 58 | CH₂CH₃ | CH₃ | CH₂CH₃ | CH₃ | 2-butyl | CH₃ | (CH₂)₄NH₂ | (S)-2-butyl |
| 59 | CH₂CH₃ | CH₃ | CH₂CH₃ | CH₃ | CH₂OH | CH₃ | (CH₂)₄NH₂ | (S)-2-butyl |
| 60 | CH₂CH₃ | CH₃ | CH₂CH₃ | CH₃ | CH₂(1H- | CH₃ | (CH₂)₄NH₂ | (S)-2-butyl |
| | | | | | indo1-3-yl) | | | |
| 61 | CH₃ | H | CH₂CH₃ | CH₃ | CH₂OH | CH₃ | (CH₂)₄NH₂ | (S)-2-butyl |
| 62 | CH₂CH₃ | CH₃ | CH₂CH₃ | CH₃ | (CH₂)₄NH₂ | CH₃ | (CH₂)₄NH₂ | (S)-2-butyl |
| 63 | CH₂CH₃ | CH₃ | CH₂CH₃ | CH₃ | CH₂OH | CH₃ | (CH₂)₄NH₂ | (S)-2-butyl |
| 64 | CH₂CH₃ | CH₃ | CH₃ | CH₂CH₃ | CH₂OH | CH₃ | (CH₂)₄NH(=NH)NHMe | (S)-2-butyl |
| 65 | CH₂CH₃ | CH₃ | CH₂CH₃ | CH₃ | CH₂OH | CH₃ | (CH₂)₄NH₂ | (S)-2-butyl |
| 66 | CH₂CH₃ | CH₃ | H | H | CH₂OH | CH₃ | (CH₂)₄NH₂ | (S)-2-butyl |
| 67 | CH₂CH₃ | CH₃ | CH₂CH₃ | CH₃ | CH₂OH | CH₃ | (CH₂)₄NH₂ | (S)-2-butyl |
| 68 | CH₂CH₃ | CH₃ | CH₂CH₃ | CH₃ | CH₂OH | CH₃ | (CH₂)₄NH₂ | (CH₂)₄NH₂ |
| 69 | CH₂CH₃ | CH₃ | CH₂CH₃ | CH₃ | CH₂OH | CH₃ | CH₃ | (CH₂)₄NH₂ |
| 70 | CH₂CH₃ | CH₃ | CH₂CH₃ | CH₃ | CH₂OH | CH₃ | (CH₂)₄NH(=NH)NH₂ | (S)-2-butyl |
| 71 | CH₂CH₃ | CH₃ | CH₂CH₃ | CH₃ | CH₂OH | CH₃ | (CH₂)₄NHCH₂Ph | (S)-2-butyl |
| 72 | CH₂CH₃ | CH₃ | CH₂CH₃ | CH₃ | CH₂OH | CH₃ | CH₂Ph(4-NH(=NH)NH₂) | (S)-2-butyl |
| 73 | CH₂CH₃ | CH₃ | CH₂CH₃ | CH₃ | CH₂OH | CH₃ | (CH₂)₄NH(=NH)NH₂ | (S)-2-butyl |
| 74 | CH₂CH₃ | CH₃ | CH₂CH₃ | CH₃ | CH₂OH | CH₃ | (CH₂)₄NH(=NH)NH₂ | (S)-2-butyl |
| 75 | CH₂CH₃ | CH₃ | CH₂CH₃ | CH₃ | CH₂OH | CH₃ | (CH₂)₄NH(=NH)NH₂ | (S)-2-butyl |

The compounds of formula (I)-(III) can be made by methods known in the art or methods described herein. Examples 1-15 below provide detailed descriptions of how compounds 1-75 were actually prepared. In some embodiments, the peptides described herein can be made in a relatively high synthetic yield. For examples, the peptides described herein can be made by a process having an overall yield (i.e., from the starting amino acids) of at least about 3% (e.g., at least about 4%, at least about 5%, at least about 6%, at least about 7%, at least about 8%, at least about 9%) and up to about 10% overall yield from the starting commercial resin.

This disclosure also features pharmaceutical compositions containing a therapeutically effective amount of at least one (e.g., two or more) of the antimicrobial peptides described herein (i.e., the compounds of formula (I)-(III)) or a pharmaceutically acceptable salt thereof as an active ingredient, as well as at least one pharmaceutically acceptable carrier (e.g., adjuvant or diluent). Examples of pharmaceutically acceptable salts include acid addition salts, e.g., a salt formed by reaction with hydrohalogen acids (such as hydrochloric acid or hydrobromic acid), mineral acids (such as sulfuric acid, phosphoric acid and nitric acid), and aliphatic, alicyclic, aromatic or heterocyclic sulfonic or carboxylic acids (such as formic acid, acetic acid, propionic acid, succinic acid, glycolic acid, lactic acid, malic acid, tartaric acid, citric acid, benzoic acid, ascorbic acid, maleic acid, hydroxymaleic acid, pyruvic acid, p-hydroxybenzoic acid, embonic acid, methanesulfonic acid, ethanesulfonic acid, hydroxyethanesulfonic acid, halobenzenesulfonic acid, trifluoroacetic acid, trifluoromethanesulfonic acid, toluenesulfonic acid, and naphthalenesulfonic acid).

The carrier in the pharmaceutical composition must be “acceptable” in the sense that it is compatible with the active ingredient of the composition (and preferably, capable of stabilizing the active ingredient) and not deleterious to the subject to be treated. One or more solubilizing agents can be utilized as pharmaceutical carriers for delivery of an active antimicrobial peptide. Examples of other carriers include colloidal silicon oxide, magnesium stearate, cellulose, sodium lauryl sulfate, and D&C Yellow #10.

The pharmaceutical composition described herein can optionally include at least one further additive selected from a disintegrating agent, binder, lubricant, flavoring agent, preservative, colorant and any mixture thereof. Examples of such and other additives can be found in “Handbook of Pharmaceutical Excipients”; Ed. A. H. Kibbe, 3rd Ed., American Pharmaceutical Association, USA and Pharmaceutical Press UK, 2000.

The pharmaceutical composition described herein can be adapted for parenteral, oral, topical, nasal, rectal, buccal, or sublingual administration or for administration via the respiratory tract, e.g., in the form of an aerosol or an air-suspended fine powder. The term “parenteral” as used herein refers to subcutaneous, intracutaneous, intravenous, intramuscular, intraarticular, intraarterial, intrasynovial, intrasternal, intrathecal, intralesional, intraperitoneal, intraocular, intra-aural, or intracranial injection, as well as any suitable infusion technique. In some embodiments, the composition can be in the form of tablets, capsules, powders, microparticles, granules, syrups, suspensions, solutions, nasal spray, transdermal patches or suppositories.

In some embodiments, the pharmaceutical composition described herein can contain an antimicrobial peptide described herein that is dissolved in an aqueous solution. For example, the composition can include a sodium chloride aqueous solution (e.g., containing 0.9 wt % of sodium chloride) to serve as a diluent.

In addition, this disclosure features a method of using an antimicrobial peptide as outlined above for treating bacterial infection or for the manufacture of a medicament for such a treatment. Additionally, this disclosure features the compounds or pharmaceutical compositions outlined above for use as a medicament. Additionally, this disclosure features the compounds or pharmaceutical compositions outlined above for use in a method of treating bacterial infection. The method can include administering to a patient in need thereof an effective amount of the pharmaceutical composition described herein. “An effective amount” refers to the amount of the pharmaceutical composition that is required to confer a therapeutic effect on the treated subject. Effective doses will vary, as recognized by those skilled in the art, depending on the types of diseases treated, route of administration, excipient usage, and the possibility of co-usage with other therapeutic treatment.

As used herein, the terms “treatment,” “treat,” and “treating” refer to reversing, alleviating, delaying the onset of, or inhibiting the progress of, a bacterial infection or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

The bacterial infection can be gram-positive bacterial infection, gram-negative bacterial infection, or mycobacterium infection. Examples of gram-positive bacteria include Clostridium difficile (C. difficile), Staphylococcus aureus, Staphylococcus epidermidis, Streptococcus pneumonia, Streptococcus pyogenes and Enterococci (e.g., Enterococcus faecalis or Enterococcus faecium). Examples of gram-negative bacteria include Escherichia coli (E. coli) and Bacteroides fragilis (B. fragilis). Without wishing to be bound by theory, it is believed that the antimicrobial peptides described herein can have improved selectivity for gram-positive bacteria versus gram-negative bacteria, improved potency and selectivity for C. difficile versus other bacteria, and/or improved pharmacokinetic properties and/or biophysical properties (such as solubility and stability). Further, without wishing to be bound by theory, it is believed that the antimicrobial peptides described herein can have both high potency and low cytotoxicity when treating a bacterial infection.

The typical dosage of the antimicrobial peptide described herein can vary within a wide range and will depend on various factors such as the individual needs of each patient and the route of administration. Exemplary daily dosages (e.g., for subcutaneous administration) can be at least about 0.5 mg (e.g., at least about 1 mg, at least about 5 mg, at least about 10 mg, or at least about 15 mg) and/or at most about 5 g (e.g., at most about 4 g, at most about 3 g, at most about 2 g, at most about 1 g, at most about 750 mg, at most about 500 mg, at least about 250 mg, at most about 100 mg, at most about 75 mg, at most about 50 mg, at most about 25 mg, or at most about 15 mg) of an antimicrobial peptide. The skilled person or physician may consider relevant variations to this dosage range and practical implementations to accommodate the situation at hand.

In some embodiments, the pharmaceutical composition described herein can be administered once daily. In some embodiments, the pharmaceutical composition can be administered more than once daily (e.g., twice daily, three times daily, or four times daily).

The contents of all publications cited herein (e.g., patents, patent application publications, and articles) are hereby incorporated by reference in their entirety. The following examples are illustrative and not intended to be limiting.

### EXAMPLES

### General Synthetic Methods

Amino acid derivatives, coupling reagents, resins, and solvents were purchased from commercial vendors, including Chem-Impex international, Bachem, Novabiochem, Combi-Blocks, Sigma-Aldrich, Fisher Scientific, and Advanced ChemTech.

The compounds described herein were prepared by standard Fmoc based solid phase peptide synthesis. Reverse phase flash and reverse phase HPLC purifications were performed on an Interchim Puriflash. In all cases, a two-solvent mobile phase was used, in which solvent A was 0.1% TFA in water and solvent B was 0.1% TFA in acetonitrile. Preparative LC columns and solvent gradients were used as described in subsequent examples. Analytical reverse phase HPLC was performed on an Agilent Technologies 1260 infinity HPLC using a Zorbax 1.8 μm C18 column (4.6×50 mm) maintained in a 40° C. column compartment. All analyses were conducted with UV detection set to 215 nm unless otherwise stated. In all cases, a two-solvent mobile phase was used, in which solvent A was 0.1% TFA in water and solvent B was 0.1% TFA in acetonitrile. Solvent gradients were used as described in subsequent examples.

LC/ESI MS was performed on a Dionex UltiMate 3000 UHPLC using a Luna 3 μM C8 column (2×50 mm) maintained in a 35° C. column compartment linked to a Dionex MSQ plus ESI mass spectrometer. All analyses were conducted in positive ion mode unless otherwise stated. In all cases, a two-solvent mobile phase was used, in which solvent A was 0.01% TFA in water and solvent B was 0.01% TFA in 95% acetonitrile/5% water. A standard gradient was used for all LC/ESI MS analyses: hold 5% B for 1 minute, then 5-100% B over 7 minutes, then hold 100% B for 1.5 minutes at 1 mL/min.

For LC-MS analysis of peptides bound to resin, a small sample of the resin was treated with 1:1 CH₂Cl₂:HFIP (200 μL) for 5 minutes in a test tube in order to cleave attached peptides. The solvent was then evaporated with a stream of nitrogen. Methanol (250 μL) was then added to the test tube and the solution was taken up in a syringe and filtered to remove the resin. The filtered solution was then submitted for LC/ESI MS analysis.

### Example 1: Synthesis of First Key Intermediate

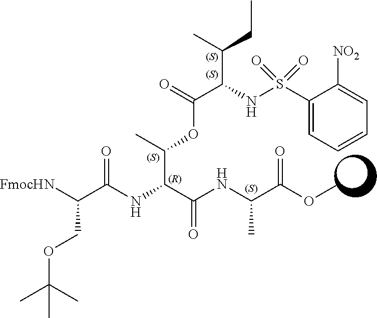

H-Ala-Trt(2-Cl)-resin (5 g, 3 mmol, pre-swelled in CH₂Cl₂) was treated with a solution of Fmoc-D-Thr-OH (2.05 g, 6 mmol), HBTU (2.28 g, 6 mmol), and NEt₃ (1.6 mL, 12 mmol) in 1:1 DMF/CH₂Cl₂ (25 mL). After the resin suspension was mixed for 1 hour, the resin was filtered and washed with DMF. Complete conversion was confirmed by a negative Kaiser test. The resin was then treated with 20% piperidine in DMF (40 mL) for two 15-minute cycles, after which the resin was washed with DMF. Finally, the resin was treated with a solution of Alloc-Osu (0.93 mL, 6 mmol) and NEt₃ (1.21 mL, 9 mmol) in 1:1 DMF/CH₂Cl₂ (25 mL). After the resin suspension was mixed for 1.5 hours, the resin was filtered and washed with DMF. Complete conversion was confirmed by a negative Kaiser test. The resin was dried and used in portions for subsequent chemistry.

The resin-bound peptide obtained above (1 mmol, pre-swelled in CH₂Cl₂) was treated with a solution of Fmoc-Ile-OH (2.12 g, 6 mmol) and DMAP (73 mg, 0.6 mmol) in 4:1 CH₂Cl₂/NMP (10 mL). The reaction was then initiated by the addition of DIC (0.93 mL, 6 mmol). After the reaction was mixed for 3 hours, the resin was filtered and washed with CH₂Cl₂. Complete conversion to the isoleucyl ester was confirmed by

LCMS. Analytical HPLC indicated <5% Ile a carbon epimerization (Gradient: 30-100% B over 10 minutes at 2 mL/min). LCMS analysis—ESI m/z observed: 610.1; required for [C₃₂H₃₉N₃O₉+H]⁺: 610.3.

The resin-bound peptide obtained above (1 mmol, pre-swelled in CH₂Cl₂) was treated with 20% piperidine in DMF for two 15-minute cycles, after which the resin was washed thoroughly with DMF. A solution of O-nitrobenzenesulfonyl chloride (663 mg, 3 mmol) and 2,4,6-collidine (1.19 mL, 9 mmol) in DMF (15 mL) was added to the resin and mixed for 2 hours. The resin was then filtered and washed with DMF and DCM. Complete conversion was confirmed by a negative Kaiser test.

The resin-bound peptide obtained above (1 mmol) was pre-swelled in CH₂Cl₂ and the solid phase reaction vessel was drained by purging with argon for 5 minutes. The resin was then treated with a solution of Pd(PPh₃)₄ (231 mg, 0.2 mmol) in CH₂Cl₂ (15 mL) followed by phenylsilane (1.5 mL, 12 mmol). The reaction was mixed for 1 hour with occasional venting to relieve pressure buildup inside the reaction vessel. The resin was filtered and washed with DCM, NMP, and DMF. Complete removal of the alloc protecting group was confirmed by LCMS. LCMS analysis—ESI m/z+observed: 489.4; required for [C₁₉H₂₈N₄O₉S +H]^{+:} 489.2.

The resin-bound peptide obtained above was then treated with a solution of Fmoc-Ser(tBu)-OH (1.14 g, 3 mmol), HOBt hydrate (462 mg, 3 mmol), and DIC (464 μL, 3 mmol) in DMF (15 mL). After the reaction was mixed overnight, the resin was filtered and washed with DMF. Complete conversion was confirmed by a negative Kaiser test.

### Example 2: Synthesis of Second Key Intermediate

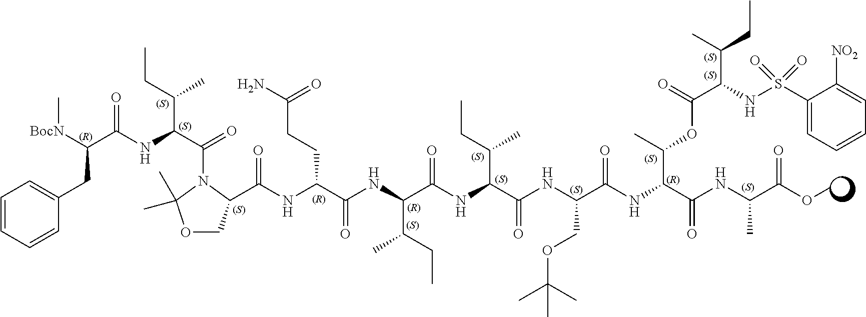

Six amino acid residues were coupled to the resin-bound peptide (1-3 mmol) synthesized in Example 1 by standard Fmoc based solid phase synthesis. Fmoc deprotections were achieved by two iterative 15 minute treatments of the resin with 20% piperidine in DMF. Couplings were achieved using one of two methods: A) 2 equivalents of an amino acid derivative, 2 equivalents of HBTU, and 2 equivalents of TEA in DMF or NMP (for Fmoc-D-Gln-OH) with 1 hour reaction time at room temperature; and B) 2 equivalents of an amino acid derivative, 2 equivalents of HOBt hydrate, and 2 equivalents of DIC in DMF with an overnight reaction time at room temperature. The following amino acid derivatives and methods were used to construct the peptide: Fmoc-Ile-OH (method A), Fmoc-D-allo-Ile-OH (Method A), Fmoc-D-Gln-OH (Method A), Fmoc-Ile-Ser(psiMe, Mepro)-OH (Method A), Boc-D-MePhe-OH (Method B). After completion of the coupling, the resin was dried and used in portions for subsequent chemistry. LCMS Analysis—ESI m/z⁺ observed: 1487.7, required for [C₇₀H₁₁₀N₁₂O₂₁S+H]^{+:} 1487.8.

### Example 3: Synthesis of Third Key Intermediate

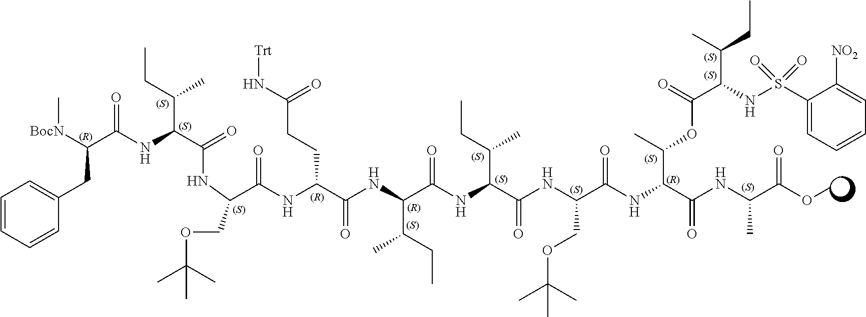

Six amino acid residues were coupled to the resin-bound peptide (0.1-0.3 mmol) synthesized in Example 1 by standard Fmoc based solid phase synthesis on a Tribute automated peptide synthesizer. Fmoc deprotections were achieved by treating the resin with 20% piperidine in DMF for successive 2 minute cycles with UV monitoring until no Fmoc cleavage product was detected. Amino acid couplings were achieved using 5 equivalents of an amino acid derivative, 5 equivalents of HBTU, and 10 equivalents of N-methylmorpholine in DMF with mixing for 30 minutes. The following amino acid derivatives and methods were used to construct the peptide: Fmoc-Ile-OH, Fmoc-D-allo-Ile-OH, Fmoc-D-Gln(Trt)-OH, Fmoc-Ser(tBu)-OH, Fmoc-Ile-OH, Boc-D-MePhe-OH. LCMS analysis—ESI m/z⁺ observed: 1745.5 Required for [C₉₀H₁₂₈N₁₂O₂₁S+H]⁺: 1745.9.

### Example 4: Synthesis of Compound 2

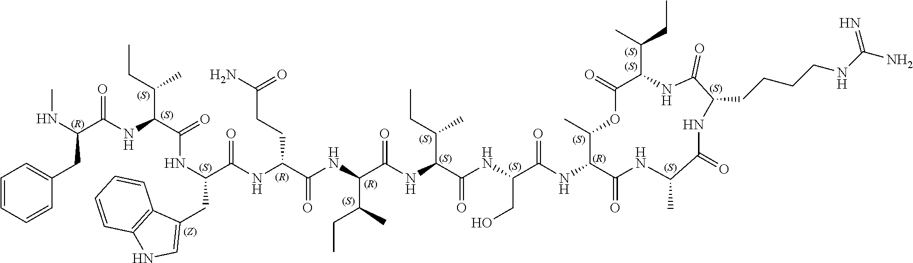

Six amino acid residues were coupled to the resin-bound peptide (0.2 mmol) synthesized in Example 1 by standard Fmoc based solid phase synthesis. Fmoc deprotections were achieved by two iterative 15-minute treatments of the resin with 20% piperidine in DMF. Couplings were conducted using 3 equivalents of an amino acid derivative, 3 equivalents of DIC, and 3 equivalents HOBt in DMF or NMP (for Fmoc-D-Gln-OH) with 2-hour reaction times at room temperature. The following amino acid derivatives were used to construct the peptide: Fmoc-Ile-OH, Fmoc-D-allo-Ile-OH, Fmoc-D-Gln-OH, Fmoc-Trp-OH, Fmoc-Ile-OH, Boc-D-MePhe-OH. LCMS Analysis—ESI m/z⁺ observed: 1647.3, required for [C₈₀H₁₁₉N₁₃O₂₂S+H]⁺: 1646.8.

The resin-bound peptide prepared above (pre-swelled in DMF) was treated with K₂CO₃ (55 mg, 0.4 mmol) and 3×1 hour cycles of 5% thiophenol in DMF. After each treatment, the resin was filtered and washed with DMF. Removal of the 2-nitrobenzene sulfonyl group was confirmed by a positive Kaiser test. The resin was next treated with a solution of Fmoc-Lys(Cbz)-OH (276 mg, 0.6 mmol) and HBTU (228 mg, 0.6 mmol) in DMF 5 mL. After the reaction was mixed for 1 hour, the resin was filtered and washed with DMF and CH₂Cl₂. Complete conversion was confirmed by a negative Kaiser test and LCMS analysis. LCMS analysis—ESI m/z+ observed: 1947.3, required for [C₁₀₃H₁₄₄N₁₄O₂₃+H]1947.1.

The resin-bound peptide prepared above was next treated with 20% piperidine in DMF for two 15-minute cycles, after which the resin was washed thoroughly with DMF and CH₂Cl₂. The peptide was then cleaved from the resin by 3×60 min treatments with 30% TFE in CH₂Cl₂, collecting the filtrate after each treatment. The filtrate was concentrated and the desired peptide was isolated by reverse phase flash chromatography

(Column: Puriflash 15 μM C18 120 g, Gradient: 40-80% B over 25 minutes at 15 mL/min). Yield—44.4 mg, 0.024 mmol. LCMS analysis—ESI m/z+ observed: 1723.9, required for [C₈₈H₁₃₄N₁₄O₂₁+H]⁺: 1724.0.

A solution of the purified peptide prepared above and NMM (9 μL, 0.09 mmol) in CH₂Cl₂(12 mL) was treated with a solution of 100 mM HATU and 300 mM HOAt in DMF (240 μL). After the reaction was mixed for 1 hour, the solvent was removed in vacuo. Complete conversion to the cyclized peptidolactone was confirmed by LCMS analysis. The crude peptide was used without purification. LCMS analysis—ESI m/z+observed: 1705.8; required for [C₈₃H₁₃₄N₁₄O₂₀+H]⁺: 1706.0.

The crude peptidolactone obtained above was dissolved in MeOH (5 mL) and acetic acid (1 mL). The solution was charged with 10% palladium on carbon (40 mg). The reaction flask was then sealed with a septum and the inner atmosphere was purged with hydrogen gas. After the hydrogenation was allowed to proceed overnight under a slight positive pressure of hydrogen, the reaction was filtered to remove palladium on carbon. Complete removal of the Cbz group was confirmed by LCMS analysis. The solvent was removed in vacuo and the lysine deprotected peptide was purified by reverse phase flash chromatography (column: Puriflash 15 μm, C_{18, 35} g; Gradient: hold at 40% B for 10 minutes, then 40-90% B over 25 minutes at 15 mL/min). Yield: 22.2 mg, 0.0132 mmol (TFA salt). LCMS analysis—ESI m/z+observed: 1571.9; required for [C₈₀ H₁₂₆N₁₄O₁₈+H]⁺: 1571.9.

A solution of the purified peptide obtained above (22.2 mg, 0.0132 mmol) and i-Pr₂NEt (6 μL, 0.03 mmol) in CH₂Cl₂ was treated with N,N′-Bis-Boc-1-guanylpyrazole (4.9 mg, 0.016 mmol) and allowed to react for 24 hours. Complete conversion was confirmed by LCMS analysis (ESI m/z+ observed: 1813.9, required for [C₉₁H₁₄₄N₁₆O₂₂+H]⁺: 1814.1). The solvent was removed in vacuo and the remaining residue was treated with TFA (2 mL). Global deprotection was allowed to proceed for 2 hours, after which the TFA was evaporated. Expulsion of CO₂ from the Boc-protectiong group on the typtophan indole nitrogen was slow under acidic conditions at room temperature, so the crude peptide was dissolved in 50% acetonitrile in water and lyophilized. LCMS analysis of the crude lyophilized solid indicated complete global deprotection. The final product peptide was isolated by reverse phase HPLC (column: Luna 5 μm C18, Gradient: 20-40% B over 20 minutes at 40 mL/min). Yield—12.2 mg, 0.00770 mmol (Di-TFA salt), 4% overall yield from starting resin. LCMS analysis—ESI m/z+observed: 1357.9, Required for: [C₆₇H₁₀₄N₁₆O₁₄+H]⁺: 1357.8.

### Example 5: Synthesis of Compound 5

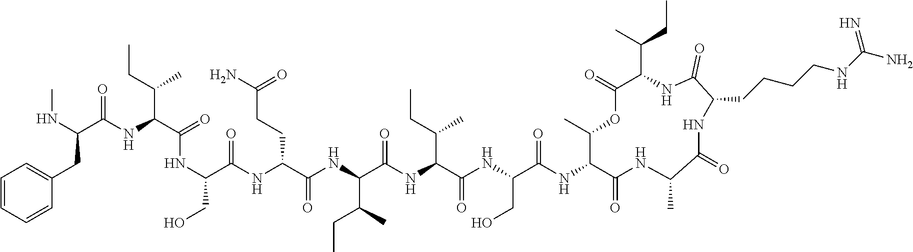 [SMILES: CC[C@H](C)[C@H](NC(=O)[C@@H](Cc1ccccc1)NC)C(=O)N[C@@H](CO)C(=O)N[C@H](CCC(N)=O)C(=O)N[C@@H](C(=O)N[C@H](C(=O)N[C@@H](CO)C(=O)N[C@H]1C(=O)N[C@@H](C)C(=O)N[C@@H](CCCCNC(=N)N)C(=O)N[C@@H]([C@@H](C)CC)C(=O)O[C@H]1C)[C@@H](C)CC)[C@@H](C)CC]

The resin-bound peptide prepared in Example 2 (0.5 mmol, preswelled in DMF) was treated with K₂CO₃ (138 mg, 1 mmol) and 3×1 hour cycles of 5% thiophenol in DMF. After each treatment, the resin was filtered and washed with DMF. Complete conversion was confirmed by LCMS and a positive Kaiser test. LCMS analysis—ESI m/z+ observed: 1302.7, required for [C₆₄H₁₀₇N₁₁O₁₇+H]⁺: 1302.8.

The resin-bound peptide obtained above was next treated with a solution of Fmoc-Lys(z)-OH (754 mg, 1.5 mmol), HBTU (570 mg, 1.5 mmol), and DIPEA (262 μL, 1.5 mmol) in DMF (5 mL). The reaction was mixed for 45 minutes, after which the resin was filtered and washed with DMF. Complete conversion was confirmed by a negative Kaiser test.

The resin-bound peptide was next treated with 20% piperidine in DMF for two 15-minute cycles, after which the resin was washed thoroughly with DMF and CH₂Cl₂. The peptide was then cleaved from the resin by 3×30 min treatments with 30% HFIP in CH₂Cl₂, collecting the filtrate after each treatment. The filtrate was concentrated and the desired peptide was isolated by reverse phase flash chromatography (Column: Puriflash 15 μM C18 40 g, Gradient: 40-80% B over 30 minutes at 15 mL/min). Yield—161 mg (0.103 mmol); LCMS analysis—ESI m/z+observed: 1564.7, required for [C₇₈H₁₂₅N₁₃O₂₀+H]+: 1564.9.

A solution of the purified branched peptide obtained above (161 mg, 0.103 mmol) and NMM (41 μL, 0.37 mmol) in DCM (50 mL) was treated with a solution composed of 100 mM HATU and 300 mM HOAt in DMF (1 mL). The cyclization reaction was allowed to proceed for 1 hour. Complete conversion was confirmed by LCMS. The reaction was concentrated and the remaining crude peptide was used without further purification. LCMS analysis—ESI m/z+observed: 1546.9, required for [C₇₈H₁₂₃N₁₃O₁₉+H]^{+:} 1546.9.

The crude cyclized peptide was dissolved in MeOH (9 mL) and acetic acid (1 mL). The solution was charged with 10% palladium on carbon (27 mg) and then the reaction flask was sealed with a septum and the inner atmosphere was purged with hydrogen gas. After the reaction as allowed to proceed under a slight positive pressure of hydrogen for 5 hours, the reaction solution was filtered to remove the palladium on carbon. The filtrate was concentrated and the desired peptide was isolated by reverse phase flash chromatography (Column: Puriflash 15 μM C18 40 g, Gradient: 40-90% B over 21 minutes at 30 mL/min). Yield—119.8 mg, 0.07851 mmol (TFA salt). LCMS analysis ESI m/z+observed: 1413.0, required for [C₇₀H₁₁₇N₁₃O₁₇+H]⁺: 1412.9.

Half of the lysine deprotected peptide (60 mg, 0.039 mmol, TFA salt) was dissolved in CH₂Cl₂ (4 mL) and treated with DIPEA (16 μL, 0.094 mmol) and N,N′-Bis-Boc-1-guanylpyrazole (15 mg, 0.047 mmol). After the reaction was allowed to proceed for 24 hours, the solvent was removed by a stream of nitrogen. The concentrated residue was then treated with TFA (4 mL) for 1 hour, after which the solvent was removed and the product was purified by reverse phase HPLC (column: Luna 5 μm C18, Gradient: 20-40% B over 20 minutes at 40 mL/min). Fractions containing the product were pooled and lyophilized to provide the product as a white powder. Yield—38 mg, 0.026 mmol (Di-TFA salt), 10% overall yield from starting resin. LCMS analysis—ESI m/z+observed: 1258.8, required for [C₅₉H₉₉N₁₅O₁₅+H]⁺: 1258.8.

### Example 6: Synthesis of Compound 6

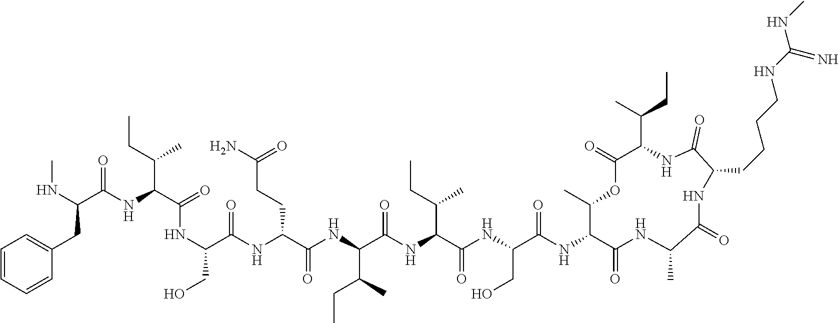 [SMILES: CC[C@H](C)[C@H](NC(=O)[C@@H](Cc1ccccc1)NC)C(=O)N[C@@H](CO)C(=O)N[C@H](CCC(N)=O)C(=O)N[C@@H](C(=O)N[C@H](C(=O)N[C@@H](CO)C(=O)N[C@H]1C(=O)N[C@@H](C)C(=O)N[C@@H](CCCCNC(=N)NC)C(=O)N[C@@H]([C@@H](C)CC)C(=O)O[C@H]1C)[C@@H](C)CC)[C@@H](C)CC]

The resin-bound peptide prepared in Example 3 (0.3 mmol paralel batches, pre-swelled in DMF) was treated 2×1 hour with 5% thiophenol in DMF (stored over K₂CO₃). After each treatment, the resin was filtered and washed with DMF. Removal of the 2-nitrobenzene sulfonyl group was confirmed by a positive Kaiser test. The resin was next treated with a solution of Fmoc-Lys(Z)-OH (452 mg, 0.9 mmol), HOBt (138 mg, 0.9 mmol), and DIC (139 μL, 0.9 mmol). After the reaction was mixed overnight, the resin was filtered and washed with DMF and CH₂Cl₂. Complete conversion was confirmed by a negative Kaiser test.

The resin-bound peptide prepared above was next treated with 20% piperidine in DMF for two 15-minute cycles, after which the resin was washed thoroughly with DMF and CH₂Cl₂. The peptide was then cleaved from the resin by 3×60 min treatments with 30% TFE in CH₂Cl₂, collecting the filtrate after each treatment. The filtrate was concentrated and the desired peptide was used without purification. LCMS analysis—ESI m/z⁺ observed: 1822.9, required for [C₉₈H₁₄₃N₁₃O₂₀+H]⁺: 1823.1.

Two identical batches of the crude peptide prepared above were combined and dissolved in DCM (30 mL) and then treated with a 0.3 M HOAt/0.1 M EDCI solution in DMF (3 mL). After 1 hour, the solvent was removed in vacuo and the product was isolated by reverse phase flash chromatography. (Column: PFC18-HQ 80g, Gradient: 60-95% B over 25 minutes, then hold 95% B for 5 minutes at 34 mL/min). Fractions containing the product were pooled, diluted with water, and then lyophilized. Yield: 653 mg, 0.36 mmol, 60% from starting resin. LCMS analysis—ESI m/z+ observed: 1805.0, required for [C₉₈H₁₄₁N₁₃O₁₉+H]⁺: 1805.1.

The purified peptide prepared above (0.36 mmol) was dissolved in MeOH (8 mL) and acetic acid (2 mL). The solution was charged with 10% palladium on carbon (76 mg). The reaction flask was then sealed with a septum and the inner atmosphere was purged with hydrogen gas. After 1 hour, the reaction was filtered to remove palladium on carbon. The filtrate was concentrated in vacuo and the remaining residue was washed with diethyl ether, providing the lysine deprotected product as white solid that was used without purification. LCMS analysis—ESI m/z⁺ observed 1670.9, required for [C₉₀H₁₃₅N₁₃O₁₇+H]⁺: 1671.0.

A portion of the peptide prepared above (assumed 0.05 mmol) was dissolved in 1:1 CH₂Cl₂/MeOH (5 mL) and treated with i-Pr₂NEt (266 μL, 1.5 mmol) and 1H-Pyrazole-1-(N-methylcarboxamideine) HCl (40 mg, 0.25 mmol). After mixing for 48 hours, the solvent was removed in vacuo and the remaining residue was used without purification. LCMS analysis—ESI m/z⁺ observed: 1727.2, required for [C₉₂H₁₃₉N₁₅O₁₇+H]⁺: 1727.1.

The peptide prepared above was treated with TFA (5 mL) and TIPS (125 μL). After 90 minutes, the reaction was concentrated in vacuo and the remaining residue was washed with diethyl ether to provide the crude product as a yellow solid. The final product was purified by reverse phase flash chromatography (column: Phenomenex Luna 5 μm C18 50 ×100 mm Gradient hold 25% B for 30 minutes then 25-95% B over 5 minutes at 40 mL/min). Fractions containing the pure product were pooled and lyophilized. Yield: 11 mg, 0.0073 mmol (Di-TFA salt), 9% from starting resin. LCMS analysis—ESI m/z⁺ observed: 1272.6, required for [C₆₀H₁₀₁N₁₅ O₁₅+H]+: 1272.8.

Example 7: Synthesis of Compound 14

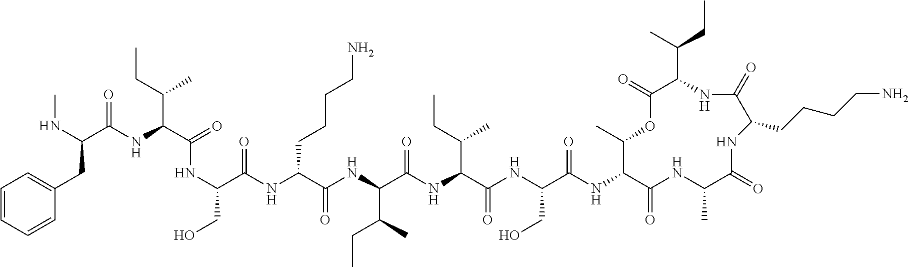 [SMILES: CC[C@H](C)[C@H](NC(=O)[C@@H](Cc1ccccc1)NC)C(=O)N[C@@H](CO)C(=O)N[C@H](CCCCN)C(=O)N[C@@H](C(=O)N[C@H](C(=O)N[C@@H](CO)C(=O)N[C@H]1C(=O)N[C@@H](C)C(=O)N[C@@H](CCCCN)C(=O)N[C@@H]([C@@H](C)CC)C(=O)O[C@H]1C)[C@@H](C)CC)[C@@H](C)CC]

Six residues were coupled to the resin-bound peptide (0.2 mmol) synthesized in Example 1 by standard Fmoc based solid phase synthesis. Fmoc deprotections were achieved by two iterative 15-minute treatments of the resin with 20% piperidine in DMF. Couplings were conducted using 3 equivalents of an amino acid derivative, 3 equivalents of DIC, and 3 equivalents HOBt in DMF or NMP (for Fmoc-D-Gln-OH) with 2-hour reaction times at room temperature. The following amino acid derivatives were used to construct the peptide: Fmoc-Ile-OH, Fmoc-D-allo-Ile-OH, Fmoc-D-Lys-OH, Fmoc-Ile-Ser(psiMe, Mepro)-OH, Boc-D-MePhe-OH. LCMS Analysis—ESI m/z+observed: 1588.1, required for [C₇₆H₁₂₂N₁₂O₂₂S+H]⁺:1587.7.

The resin-bound peptide obtained above (pre-swelled in DMF) was treated with K₂CO₃ (55 mg, 0.4 mmol) and 3×1 hour cycles of 5% thiophenol in DMF. After each treatment, the resin was filtered and washed with DMF. Removal of the 2-nitrobenzene sulfonyl group was confirmed by a positive Kaiser test. The resin was next treated with a solution of Fmoc-Lys(Cbz)-OH (276 mg, 0.6 mmol) and HBTU (228 mg, 0.6 mmol) in DMF 5 mL. After the reaction was mixed for 1 hour, the resin was filtered and washed with DMF and CH₂Cl₂. Complete conversion was confirmed by a negative Kaiser test and LCMS analysis. LCMS analysis—ESI m/z+ observed: 1888.1, required for [C₉₉H₁₄₇N₁₃O₂₃+H]⁺: 1888.1.

The resin-bound peptide obtained above was next treated with 20% piperidine in DMF for two 15-minute cycles, after which the resin was washed thoroughly with DMF and CH₂Cl₂. The peptide was then cleaved from the resin by 3×60 min treatments with 30% TFE in CH₂Cl₂, collecting the filtrate after each treatment. The filtrate was concentrated and the desired peptide was isolated by reverse phase flash chromatography (Column: Puriflash 15 μM C18 120 g, Gradient: 40-80% B over 25 minutes at 15 mL/min). Yield—26.1 mg, 0.0156 mmol. LCMS analysis—ESI m/z+ observed: 1665.1, required for [C₈₄H₁₃₇N₁₃O₂₁+H]⁺: 1665.0.

A solution of the purified peptide and i-Pr₂NEt (9 μL, 0.05 mmol) in CH₂Cl₂ (7.25 mL) was treated with a solution of 100 mM HBTU DMF (145 μL). After the reaction was mixed for 1 hour, the solvent was removed in vacuo. Complete conversion to the cyclized peptidolactone was confirmed by LCMS analysis. The crude peptidolactone was used without purification. LCMS analysis—ESI m/z+ observed: 1646.8; required for [C₈₄H₁₃₅N₁₃O₂₀+H]+: 1647.0.

The crude peptidolactone obtained above was dissolved in MeOH (2 mL) and acetic acid (200 μL). The solution was charged with 10% palladium on carbon (40 mg). The reaction flask was then sealed with a septum and the inner atmosphere was purged with hydrogen gas. After the hydrogenation was allowed to proceed for 90 minutes under a slight positive pressure of hydrogen, the reaction mixture was filtered to remove palladium on carbon. Complete removal of the Cbz group was confirmed by LCMS analysis (ESI m/z+observed: 1512.90; Required for [C₇₆H₁₂₉N₁₃O₁₈+H]⁺: 1512.97). The filtered peptide solution was concentrated in vacuo and treated with TFA (2 mL) for 1 hour, after which the TFA was evaporated. The final product peptide was isolated by reverse phase HPLC (column: Luna 5 μm C18, Gradient: 20-40% B over 20 minutes at 40 mL/min). Yield—9.7 mg, 0.0062 mmol (tri-TFA salt), 3% overall yield from starting resin. LCMS analysis—ESI m/z+observed: 1216.8; required for [C₅₉H1o1N13014 +H]⁺: 1216.8.

### Example 8: Synthesis of Compound 34

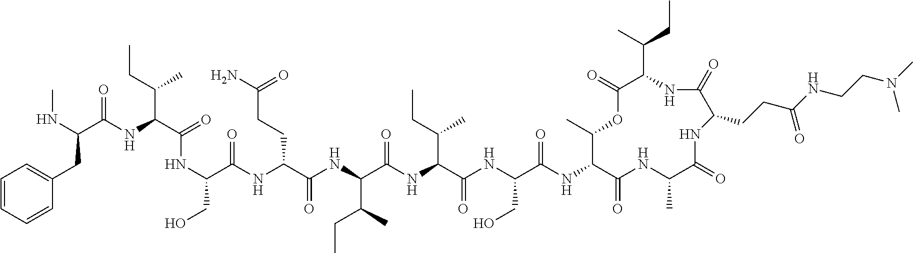 [SMILES: CC[C@H](C)[C@H](NC(=O)[C@@H](Cc1ccccc1)NC)C(=O)N[C@@H](CO)C(=O)N[C@H](CCC(N)=O)C(=O)N[C@@H](C(=O)N[C@H](C(=O)N[C@@H](CO)C(=O)N[C@H]1C(=O)N[C@@H](C)C(=O)N[C@@H](CCC(=O)NCCN(C)C)C(=O)N[C@@H]([C@@H](C)CC)C(=O)O[C@H]1C)[C@@H](C)CC)[C@@H](C)CC]

The resin-bound peptide prepared in Example 2 (0.32 mmol) was treated K₂CO₃ (88 mg, 64 mmol) and 3×1 hour cycles of 5% thiophenol in DMF. After each treatment, the resin was filtered and washed with DMF. Complete conversion was confirmed by LCMS and a positive Kaiser test. LCMS analysis—ESI m/z+observed: 1302.7, required for [C₆₄H₁₀₇N₁₁O₁₇+H]⁺:1302.8.

The resin was then treated with a solution of Fmoc-Glu(OBzl)—OH (294 mg, 0.64 mmol), HBTU (99 mg, 0.64 mmol), and TEA (200 μL, 1.28 mmol) in DMF (5 mL). After the reaction was mixed for 60 minutes, the resin was filtered and washed with DMF. Complete conversion was confirmed by a negative Kaiser test.

The resin-bound peptide obtained above was next treated with 20% piperidine in DMF for two 15 minute cycles, after which the resin was washed thoroughly with DMF and CH₂Cl₂. The peptide was then cleaved from the resin by 3×30-minute treatments with 30% HFIP in CH₂Cl₂, collecting the filtrate after each treatment. The filtrate was concentrated and the desired peptide was isolated by reverse phase flash chromatography. (Column: Puriflash 15 μM C18 120 g, Gradient: 30-70% B over 20 minutes at 50 mL/min). Yield—88 mg (0.058 mmol). LCMS analysis—ESI m/z⁺ observed: 1521.6, required for [C₇₆H₁₂₀N₁₂O₂₀+H]⁺: 1521.9.

The purified peptide obtained above (88 mg, 0.058 mmol) was dissolved in CH₂Cl₂ (50 mL) and DMF (15 mL) with i-Pr₂NEt (36 μL, 0.208 mmol) and cooled to 0° C. The solution was then treated with a solution of HBTU (26 mg, 0.069 mmol) in DMF (1 mL) and allowed to react for 30 minutes. Complete cyclization conversion was confirmed by LCMS analysis. The reaction solution was transferred to a separatory funnel and extracted with aqueous NaHCO₃ and Brine. The organic phase was collected and dried over magnesium sulfate, after which the solution was filtered and concentrated in vacuo. The crude product residue was used without purification. LCMS analysis—ESI m/z⁺observed: 1504.2, required for [C₇₆H₁₁₈N₁₂O₁₉+H]⁺: 1503.9.

A solution of the crude cyclized peptide in MeOH (10 mL) was charged with 10% palladium on carbon (31 mg). The reaction flask was then sealed with a septum and purged with hydrogen gas. After the reaction was allowed to proceed under a slight positive pressure of hydrogen for one hour, the reaction was filtered to remove palladium on carbon. Complete conversion was confirmed by LCMS analysis. The filtered reaction solution was concentrated in vacuo and the remaining oily residue was taken up in 1:1 acetonitrile/water, which produced a white precipitate. The precipitate was removed by filtration and the filtrate containing the desired product was concentrated in vacuo. The crude product residue was used without purification. LCMS analysis—ESI m/z⁺ observed: 1413.8, required for [C₆₉H₁₁₂N₁₂O₁₉+H]⁺: 1413.8.

The crude Glu-deprotected peptide was either treated with TFA in order to achieve global deprotection or alternatively, a solution of the peptide (26 mg, 0.018 mmol) in CH₂Cl₂ (1 mL) and i-Pr₂NEt (12 μL, 0.066 mmol) in DCM (1 mL) was treated with 100 mM HBTU in DMF (220 μL, 0.022 mmol) and allowed to react for 15 minutes. N,N-dimethylethylenediamine (4 μL, 0.036 mmol) was then added. After 1 hour, the reaction was diluted with ethyl acetate (˜15 mL) and extracted with aqueous sodium bicarbonate and brine. The organic phase was dried over magnesium sulfate and then filtered and concentrated in vacuo. After the resulting residue was treated with TFA and allowed to react for 1 hour, the solvent was removed and the product was purified by preparative reverse phase HPLC (Column: Luna 5 μm C18, 100×30 mm; Gradient 20-40% B over 20 minutes at 40 mL/min). Fractions containing the product were combined and lyophilized to provide the product as a white powder. Yield: 11.7 mg, 0.00772 mmol (Di-TFA salt), 2% overall yield from starting resin. LCMS analysis—ESI m/z⁺ observed: 1287.7, required for [C₆₁H₁₀₂N₁₄O₁₆+H]⁺: 1287.8.

### Example 9: Synthesis of Compound 70

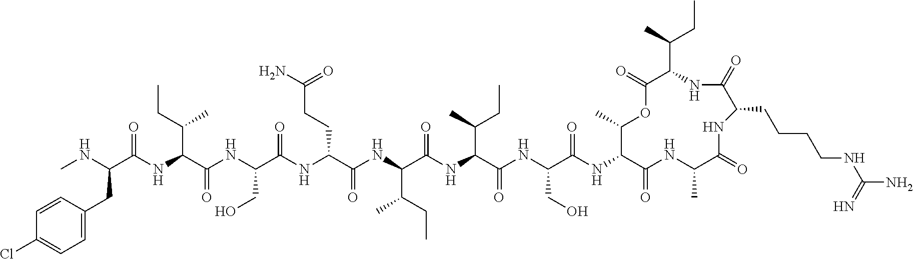 [SMILES: CC[C@H](C)[C@H](NC(=O)[C@@H](Cc1ccc(Cl)cc1)NC)C(=O)N[C@@H](CO)C(=O)N[C@H](CCC(N)=O)C(=O)N[C@@H](C(=O)N[C@H](C(=O)N[C@@H](CO)C(=O)N[C@H]1C(=O)N[C@@H](C)C(=O)N[C@@H](CCCCNC(=N)N)C(=O)N[C@@H]([C@@H](C)CC)C(=O)O[C@H]1C)[C@@H](C)CC)[C@@H](C)CC]

Compound 70 was synthesized in the same manner as in Example 5, substituting Boc-D-MePhe(4-Cl)—OH for Boc-D-MePhe-OH for the installation of the amino acid at position 1. The final product was purified by reverse phase flash chromatography (Column PF-15CN 40 g; Gradient: 20-60% B over 25 min at 27 mL/min). Fractions containing the purified product were pooled and lyophilized. Yield: 143 mg, 0.094 mmol (Di-TFA salt), 31% from starting resin. LCMS analysis—ESI m/z⁺ observed: 1292.8, required for [C₅₉H₉₈ClN₁₅O₁₅+H]⁺: 1292.7.

### Example 10: Synthesis of Compound 71

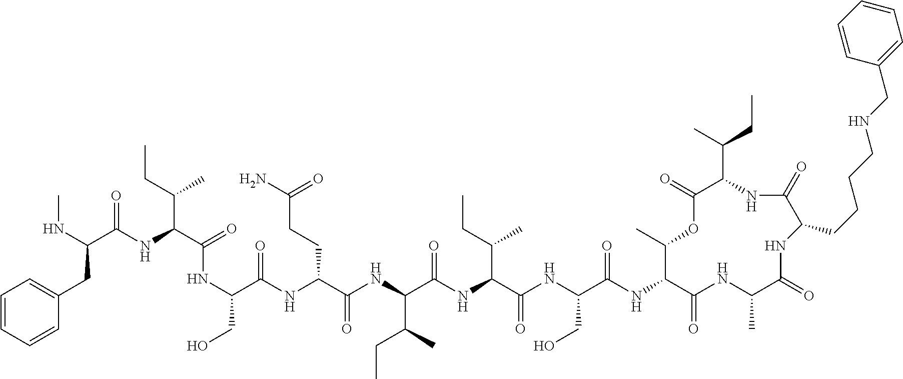 [SMILES: CC[C@H](C)[C@H](NC(=O)[C@@H](Cc1ccccc1)NC)C(=O)N[C@@H](CO)C(=O)N[C@H](CCC(N)=O)C(=O)N[C@@H](C(=O)N[C@H](C(=O)N[C@@H](CO)C(=O)N[C@H]1C(=O)N[C@@H](C)C(=O)N[C@@H](CCCCNCc2ccccc2)C(=O)N[C@@H]([C@@H](C)CC)C(=O)O[C@H]1C)[C@@H](C)CC)[C@@H](C)CC]

The resin-bound peptide prepared in Example 3 (0.3 mmol paralel batches, pre-swelled in DMF) was treated 2×1 hour with 5% thiophenol in DMF (stored over K₂CO₃). After each treatment, the resin was filtered and washed with DMF. Removal of the 2-nitrobenzene sulfonyl group was confirmed by a positive Kaiser test. The resin was next treated with a solution of Fmoc-Lys(Z)-OH (452 mg, 0.9 mmol), HOBt (138 mg, 0.9 mmol), and DIC (139 μL, 0.9 mmol). After the reaction was mixed overnight, the resin was filtered and washed with DMF and CH₂Cl₂. Complete conversion was confirmed by a negative Kaiser test.

The resin-bound peptide prepared above was next treated with 20% piperidine in DMF for two 15-minute cycles, after which the resin was washed thoroughly with DMF and CH₂Cl₂. The peptide was then cleaved from the resin by 3×60 min treatments with 30% TFE in CH₂Cl₂, collecting the filtrate after each treatment. The filtrate was concentrated and the desired peptide was used without purification. LCMS analysis—ESI m/z⁺ observed: 1822.9, required for [C₉8H₁₄₃N₁₃O₂₀+H]^{+:} 1823.1.

Two identical batches of the crude peptide prepared above were combined and dissolved in DCM (30 mL) and then treated with a 0.3 M HOAt/0.1 M EDCI solution in DMF (3 mL). After 1 hour, the solvent was removed in vacuo and the product was isolated by reverse phase flash chromatography. (Column: PFC18-HQ 80g, Gradient: 60-95% B over 25 minutes, then hold 95% B for 5 minutes at 34 mL/min). Fractions containing the product were pooled, diluted with water, and then lyophilized. Yield: 653 mg, 0.36 mmol, 60% from starting resin. LCMS analysis—ESI m/z+observed: 1805.0, required for [C₉₈H₁₄₁N₁₃O₁₉+H]⁺: 1805.1.

The purified peptide prepared above (0.36 mmol) was dissolved in MeOH (8 mL) and acetic acid (2 mL). The solution was charged with 10% palladium on carbon (76 mg). The reaction flask was then sealed with a septum and the inner atmosphere was purged with hydrogen gas. After 1 hour, the reaction was filtered to remove palladium on carbon. The filtrate was concentrated in vacuo and the remaining residue was washed with diethyl ether, providing the lysine deprotected product as white solid that was used without purification. LCMS analysis—ESI m/z⁺ observed 1670.9, required for [C₉₀H₁₃₅N₁₃O₁₇+l H]⁺: 1671.0.

A portion of the crude peptide prepared above (assumed 0.1 mmol) was dissolved in a mixture of acetonitrile (8 mL) and acetic acid (1 mL). The solution was charged with benzaldehyde (1 mL, 10 mmol) and sodium triacetoxyborohydride (850 mg, 4 mmol). After mixing for 48 hours, the reaction was quenched with saturated aqueous ammonium chloride and then lyophilized. The crude solid was used without purification. LCMS analysis—ESI m/z⁺ observed: 1761.1 required for [C97H141N13O17+H]+: 1761.1

The crude peptide prepared above was treated with 95:5:2.5 TFA/water/TIS (10 mL). After 2 hours, the reaction was concentrated in vacuo and the remaining residue was washed with diethyl ether to provide the crude product as a yellow solid. The product was purified by preparative HPLC (column: Phenomenex Luna 5 μm C18 50×100 mm, Gradient 20-40% B over 20 minutes at 40 mL/min). Fractions containing the pure product were pooled and lyophilized. Yield: 29 mg, 0.019 mmol (Di-TFA salt), 11% from starting resin. LCMS analysis—ESI m/z⁺ observed: 1306.9, required for [C₆₅H₁₀₃N₁₃O₁₅+H]⁺: 1306.8.

### Example 11: Synthesis of Compound 72

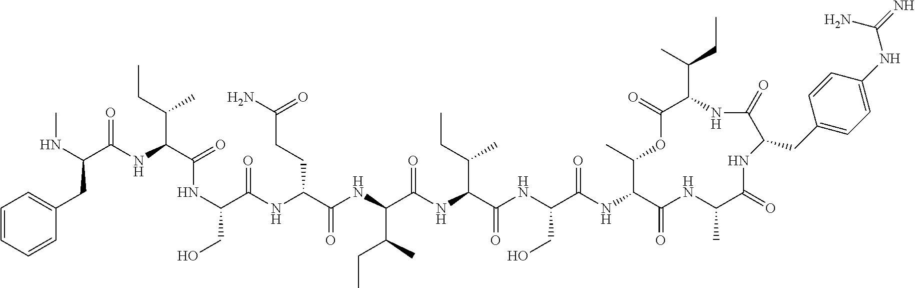 [SMILES: CC[C@H](C)[C@H](NC(=O)[C@@H](Cc1ccccc1)NC)C(=O)N[C@@H](CO)C(=O)N[C@H](CCC(N)=O)C(=O)N[C@@H](C(=O)N[C@H](C(=O)N[C@@H](CO)C(=O)N[C@H]1C(=O)N[C@@H](C)C(=O)N[C@@H](Cc2ccc(NC(=N)N)cc2)C(=O)N[C@@H]([C@@H](C)CC)C(=O)O[C@H]1C)[C@@H](C)CC)[C@@H](C)CC]

The resin-bound peptide prepared in Example 3 (0.3 mmol, pre-swelled in DMF) was treated 2×1 hour with 5% thiophenol in DMF (stored over K₂CO₃). After each treatment, the resin was filtered and washed with DMF. Removal of the 2-nitrobenzene sulfonyl group was confirmed by a positive Kaiser test. The resin was next treated with a solution Fmoc-Phe(4-guanidino-boc2)-OH (580 mg, 0.9 mmol), HATU (344 mg, 0.9 mmol), and i-Pr₂NEt (315 μL, 1.8 mmol). After the reaction was mixed for 2 hours, the resin was filtered and washed with DMF and CH₂Cl₂. Complete conversion was confirmed by a negative Kaiser test.

The resin-bound peptide prepared above was next treated with 20% piperidine in DMF for two 15-minute cycles, after which the resin was washed thoroughly with DMF and CH₂C1₂. The peptide was then cleaved from the resin by 3×60 min treatments with 30% TFE in CH₂Cl₂, collecting the filtrate after each treatment. The filtrate was concentrated and the desired peptide was isolated by reverse phase flash chromatography (Column: Puriflash 15 μM C18 120 g, Gradient: 40-80% B over 25 minutes at 47 mL/min). Two products were isolated corresponding to the desired product as well as a side-product with one Boc group cleaved from the guanidine phenylalanine side chain. Fractions containing each product were pooled and lyophilized. Yields: desired product—135 mg, 0.060 mmol, 20% from starting resin, side product—65 mg, 0.035 mmol, 12% from starting resin. LCMS analyses: Desired product—ESI m/z⁺ observed: 1965.0, required for [C₁₀₄H₁₅₃N₁₅O₂₂+H]⁺: 1965.1. Side-product—ESI m/z⁺ observed: 1965.0, required for [C₉₉H₁₄₅N₁₅O₂₀+H]⁺: 1865.1.

The desired product prepared above was dissolved in CH₂C1₂ (6 mL) and treated with a solution EDCI (0.1 M) and HOAt (0.3 M) in DMF (0.72 mL). After 1 hour, the solvent was removed and the remaining residue was used without purification. LCMS analysis—ESI m/z⁺ observed: 1947.1, required for [C₁₀₄H₁₅₁N₁₅O₂₁+H]⁺: 1947.1. The side-product prepared above was dissolved in CH₂Cl₂ (4 mL) and treated with a solution EDCI (0.1 M) and HOAt (0.3 M) in DMF (0.48 mL). After 1 hour, the solvent was removed and the remaining residue was used without purification. LCMS analysis ESI m/z⁺ observed: 1847.1, required for [C₉₉H₁₄₃N₁₅O₁₉+H]⁺: 1747.1.

The two peptides prepared above were combined and treated with a cocktail containing 95:5:2.5 TFA/H2O/TIS (10 mL). After 90 minutes, the reaction was concentrated in vacuo and the remaining residue was washed with diethyl ether to provide the crude product as a yellow solid. The final product was isolated by reverse phase flash chromatography (Column PF-15CN 40 g; Gradient: 20-60% B over 25 min at 27 mL/min). Fractions containing the purified product were pooled and lyophilized. Yield: 76 mg, 0.05 mmol (Di-TFA salt), 17% from starting resin. LCMS analysis—ESI m/z⁺ observed: 1292.9, required for [C₆₂H₉₇N₁₅O₁₅+H]^{+:} 1292.7.

### Example 12: Synthesis of Compound 73

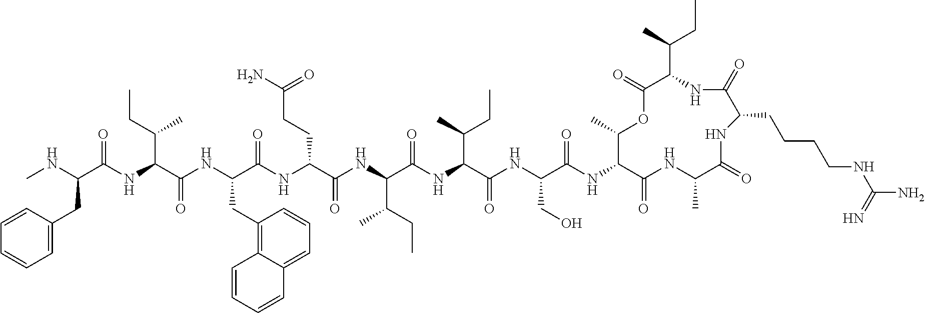 [SMILES: CC[C@H](C)[C@H](NC(=O)[C@@H](Cc1ccccc1)NC)C(=O)N[C@@H](Cc1cccc2ccccc12)C(=O)N[C@H](CCC(N)=O)C(=O)N[C@@H](C(=O)N[C@H](C(=O)N[C@@H](CO)C(=O)N[C@H]1C(=O)N[C@@H](C)C(=O)N[C@@H](CCCCNC(=N)N)C(=O)N[C@@H]([C@@H](C)CC)C(=O)O[C@H]1C)[C@@H](C)CC)[C@@H](C)CC]

Compound 73 was synthesized in the same manner as in Example 4, substituting Fmoc-1-Nal-OH for Fmoc-Trp(Boc)-OH for the installation of the amino acid in position 3. The final product was purified by reverse phase flash chromatography (Column PF-15CN 40 g; Gradient: 20-35% B over 20 min at 27 mL/min). Fractions containing the purified product were pooled and lyophilized. Yield: 20 mg, 0.013 mmol (Di-TFA salt), 7% from starting resin. LCMS analysis—ESI m/z+ observed: 1368.9, required for [C₆₉H₁₀₅N₁₅O₁₄+H]⁺: 1368.8.

### Example 13: Synthesis of Compound 74

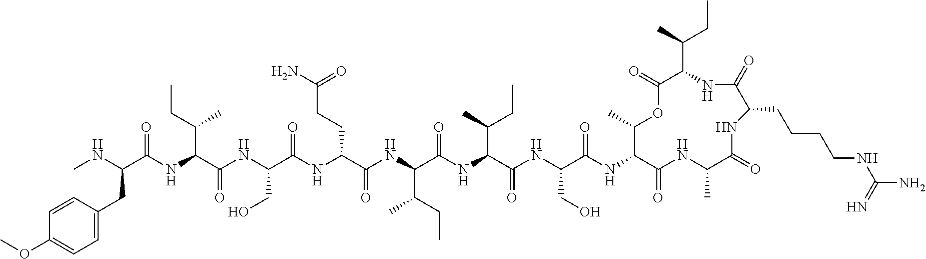 [SMILES: CC[C@H](C)[C@H](NC(=O)[C@@H](Cc1ccc(OC)cc1)NC)C(=O)N[C@@H](CO)C(=O)N[C@H](CCC(N)=O)C(=O)N[C@@H](C(=O)N[C@H](C(=O)N[C@@H](CO)C(=O)N[C@H]1C(=O)N[C@@H](C)C(=O)N[C@@H](CCCCNC(=N)N)C(=O)N[C@@H]([C@@H](C)CC)C(=O)O[C@H]1C)[C@@H](C)CC)[C@@H](C)CC]

Compound 74 was synthesized in the same manner as in Example 5, substituting Boc-D-MeTyr(Me)-OH for Boc-D-MePhe-OH for the installation of the amino acid at position 1. The final product was purified by reverse phase flash chromatography (column: Phenomenex Luna 5 μm C18 50×100 mm Gradient: 20-38% B over 20 minutes at 40 mL/min). Fractions containing the purified product were pooled and lyophilized. Yield: 54 mg, 0.036 mmol (Di-TFA salt), 18% from starting resin. LCMS analysis—ESI m/z+observed: 1288.9, required for [C₆₀H₁₀₁N₁₅O₁₆+H]⁺: 1288.8.

### Example 14: Synthesis of Compound 75

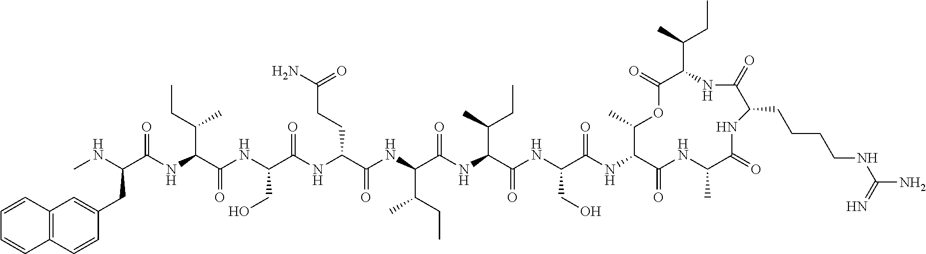 [SMILES: CC[C@H](C)[C@H](NC(=O)[C@@H](Cc1ccc2ccccc2c1)NC)C(=O)N[C@@H](CO)C(=O)N[C@H](CCC(N)=O)C(=O)N[C@@H](C(=O)N[C@H](C(=O)N[C@@H](CO)C(=O)N[C@H]1C(=O)N[C@@H](C)C(=O)N[C@@H](CCCCNC(=N)N)C(=O)N[C@@H]([C@@H](C)CC)C(=O)O[C@H]1C)[C@@H](C)CC)[C@@H](C)CC]

Compound 75 was synthesized in the same manner as in Example 5, substituting Boc-D-2-MeNal-OH for Boc-D-MePhe-OH for the installation of the amino acid at position 1. The final product was purified by reverse phase flash chromatography

(Column PF-15CN 40 g; Gradient: 15-35% B over 20 min at 27 mL/min). Fractions containing the purified product were pooled and lyophilized. Yield: 108 mg, 0.070 mmol (Di-TFA salt), 35% from starting resin. LCMS analysis—ESI m/z+observed: 1309.0, required for [C₆₃H₁₀₁N₁₅O₁₅+H]⁺: 1308.8.

### Example 15: Synthesis of Compounds 1, 3, 4, 7-13, 15-33, and 35-69

Compounds 1, 3, 4, 7-13, 15-33, and 35-69 were synthesized following the methods described in Examples 3-14 when using appropriate amino acids as starting materials.

The observed and calculated MS data (i.e., M+H) of Compounds 1-75 are summarized in Table 3 below.

TABLE 3

| Compound | Calculated | Observed |
|---|---|---|
| No. | M + H | M + H |
| 1 | 1299.8 | 1300.1 |
| 2 | 1357.8 | 1357.9 |
| 3 | 1326.8 | 1327.0 |
| 4 | 1236.7 | 1236.9 |
| 5 | 1258.8 | 1258.8 |
| 6 | 1272.8 | 1273.0 |
| 7 | 1315.8 | 1316.0 |
| 8 | 1257.8 | 1258.0 |
| 9 | 1230.8 | 1231.0 |
| 10 | 1216.7 | 1217.0 |
| 11 | 1258.7 | 1259.0 |
| 12 | 1216.7 | 1217.0 |
| 13 | 1274.7 | 1275.0 |
| 14 | 1216.8 | 1216.8 |
| 15 | 1216.7 | 1217.0 |
| 16 | 1411.8 | 1412.0 |
| 17 | 1200.7 | 1201.0 |
| 18 | 1242.8 | 1243.0 |
| 19 | 1201.8 | 1202.0 |
| 20 | 1188.7 | 1188.8 |
| 21 | 1230.7 | 1230.8 |
| 22 | 1244.7 | 1245.0 |
| 23 | 1159.7 | 1159.9 |
| 24 | 1321.8 | 1322.0 |
| 25 | 1341.8 | 1342.0 |
| 26 | 1216.7 | 1216.9 |
| 27 | 1202.7 | 1203.0 |
| 28 | 1258.7 | 1258.8 |
| 29 | 1273.7 | 1273.9 |
| 30 | 1258.8 | 1259.1 |
| 31 | 1244.7 | 1245.1 |
| 32 | 1230.7 | 1231.0 |
| 33 | 1216.7 | 1217.0 |
| 34 | 1287.8 | 1287.7 |
| 35 | 1259.7 | 1260.0 |
| 36 | 1244.8 | 1245.1 |
| 37 | 1216.7 | 1217.0 |
| 38 | 1244.7 | 1244.8 |
| 39 | 1259.7 | 1259.9 |
| 40 | 1159.7 | 1159.8 |
| 41 | 1225.7 | 1225.8 |
| 42 | 1202.7 | 1203.0 |
| 43 | 1289.7 | 1290.0 |
| 44 | 1289.7 | 1289.9 |
| 45 | 1230.7 | 1230.8 |
| 46 | 1245.7 | 1245.9 |
| 47 | 1289.7 | 1290.0 |
| 48 | 1230.8 | 1231.0 |
| 49 | 1438.8 | 1439.1 |
| 50 | 1168.7 | 1169.0 |
| 51 | 1289.7 | 1290.0 |
| 52 | 1159.7 | 1159.8 |
| 53 | 1174.6 | 1174.9 |
| 54 | 1200.7 | 1200.9 |
| 55 | 1230.7 | 1231.0 |
| 56 | 1217.7 | 1217.9 |
| 57 | 1225.7 | 1225.9 |
| 58 | 1242.8 | 1243.0 |
| 59 | 1241.7 | 1242.0 |
| 60 | 1315.8 | 1316.1 |
| 61 | 1174.7 | 1174.9 |
| 62 | 1257.8 | 1258.0 |
| 63 | 1140.7 | 1140.9 |
| 64 | 1258.7 | 1259.0 |
| 65 | 1287.8 | 1288.0 |
| 66 | 1174.7 | 1174.9 |
| 67 | 1244.7 | 1244.9 |
| 68 | 1231.7 | 1232.0 |
| 69 | 1174.7 | 1175.0 |
| 70 | 1292.7 | 1292.8 |
| 71 | 1306.8 | 1306.9 |
| 72 | 1292.7 | 1292.9 |
| 73 | 1368.8 | 1368.9 |
| 74 | 1288.8 | 1288.9 |
| 75 | 1308.8 | 1309.0 |

### Example 16: Synthesis of Teixobactin

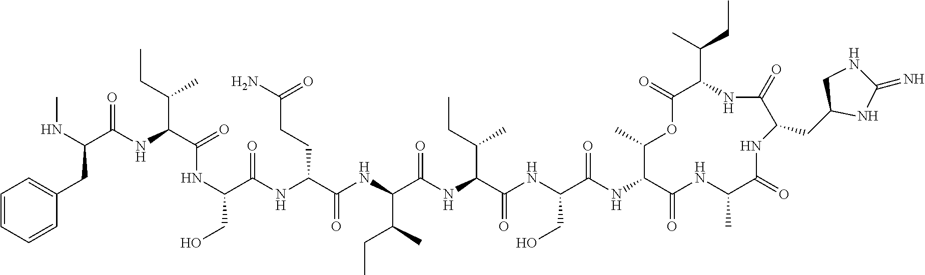 [SMILES: CC[C@H](C)[C@H](NC(=O)[C@@H](Cc1ccccc1)NC)C(=O)N[C@@H](CO)C(=O)N[C@H](CCC(N)=O)C(=O)N[C@@H](C(=O)N[C@H](C(=O)N[C@@H](CO)C(=O)N[C@H]1C(=O)N[C@@H](C)C(=O)N[C@@H](C[C@H]2CNC(=N)N2)C(=O)N[C@@H]([C@@H](C)CC)C(=O)O[C@H]1C)[C@@H](C)CC)[C@@H](C)CC]

The resin-bound peptide prepared in Example 2 (0.05 mmol, preswelled in DMF) was treated with K2CO3 (28 mg, 0.2 mmol) and 3×1 hour cycles of 5% thiophenol in DMF. After each treatment, the resin was filtered and washed with DMF. Complete conversion was confirmed by LCMS and a positive Kaiser test. LCMS Analysis—ESI m/z⁺ observed: 1302.81, required for [C₆₄H₁₀₇N₁₁O₁₇+H]⁺: 1302.79.

The resin-bound peptide was then treated with a solution of Fmoc-allo-End(Cbz)₂-OH (50 mg, 0.075 mmol) and HOAt (21 mg, 0.15 mmol) in DMF (2 mL). The resin suspension was mixed and then the coupling reaction was initiated by the addition of HATU (29 mg, 0.075 mmol) and DIPEA (26 μL, 0.15 mmol) in quick succession. The reaction was mixed for 2 hours, after which the resin was filtered and washed with DMF. Complete conversion was confirmed by LCMS. ESI m/z+ observed: 1947.51 required for [C₁₀₁H₁₃₉N₁₅O₂₄+H]+: 1948.02.

The resin-bound peptide was treated with 20% piperidine in DMF for two 15-minute cycles, after which the resin was washed thoroughly with DMF and CH₂Cl₂. Then, the peptide was cleaved from the resin by 3×30 min treatments with 30% HFIP in CH₂Cl₂, collecting the filtrate after each treatment. LCMS indicated that the crude cleaved product was a mixture of mono-Cbz and di-Cbz allo-enduracididine protected peptides. The combined filtrates were concentrated and then the two products were cleanly isolated by reverse phase HPLC (column: Luna 5 μM C18, Gradient 40-77% B over 20 min at 40 mL/min). Yields: mono-Cbz product: 22.7 mg (0.0142 mmol), di-Cbz product: 9.9 mg (0.0057 mmol). LCMS analysis—mono-Cbz product ESI m/z+observed: 1590.74, required for [C₇₈H₁₂₃N₁₅O₂₀+H]⁺: 1590.91; di-Cbz product ESI m/z+observed: 1724.83, required for [C₈₆H₁₂₉N₁₅O₂₂+H]⁺:1724.95.

A stirring solution of the mono-Cbz allo-enduracididine protected peptide (22.7 mg, 0.0142 mmol) in CH₂Cl₂ (2.9 mL) was treated with a 0.15 M solution of HOAt (286 μL, 0.0429 mmol) in DMF, followed by a 0.05 M solution of HATU (286 μL, 0.143 mmol) in DMF, and lastly a 0.15 M solution of 4-methylmorpholine (286 μL, 0.0429 mmol) in CH₂Cl₂. The reaction was allowed to proceed for 1 hour, after which LCMS indicated complete conversion of the starting material to the cyclized product. The solvent was removed in vacuo and the crude product was used without purification. LCMS analysis—ESI m/z+observed: 1572.78, required for [C₇₈H₁₂₁N₁₅O₁₉+H]+: 1572.90.

The crude cyclized peptide was dissolved in 2 mL MeOH with 200 μL, acetic acid. The solution was charged with 10% Pd/C (12 mg) and then the reaction vessel was capped with a septum and the atmosphere was purged with hydrogen gas. The hydrogenation reaction was allowed to proceed for 30 minutes under a slight positive pressure of hydrogen, after which LCMS indicated clean conversion to the allo-enduracididine deprotected intermediate. The reaction was then filtered and concentrated in vacuo. The crude residue was used without purification. LCMS analysis: ESI m/z⁺ observed: 1438.84, required for [C₇₀H₁₁₅N₁₅O₁₇+H]⁺:1438.87.

The partially deprotected crude peptide was then treated with TFA (2 mL) at room temperature for 1.5 hours, after which LCMS indicated complete global deprotection. The solvent was removed in vacuo and the final product was purified by HPLC (column Luna 5 μM C18, Gradient: 20-40% B over 20 min at 40 mL/min). Fractions containing the product were combined and lyophilized to provide the product as a white powder. Yield: 8.1 mg (0.0055 mmol, Di-TFA salt). LCMS analysis—ESI m/z+observed: 1242.73 required for [C₅₈H₉₅N₁₅O₁₅+H]⁺: 1242.72.

### Example 17: Broth Microdilution Assay for Determining the Minimum Inhibitory Concentration (MIC)

Certain compounds 1-75 obtained above were tested for their efficacy in inhibiting bacterial growth as outlined below.

### 17a. E.coli and S.aureus

This assay was used to determine the minimum concentration of a test compound (i.e., an antimicrobial peptide described herein) able to inhibit the growth of a bacterial strain of interest. This is a standard method for measuring and comparing the potency of peptide-based antibacterial agents (Steinberg et al., Antimicrobial Agents and Chemotherapy 1997, 41 (8) 1738 - 1742 and Wiegand et al., Nature Protocols 2008, 3, 163-175).

The MIC is defined as the lowest concentration of a test compound that is able to completely inhibit the growth of a bacterial culture with an inoculum of ˜5×10⁵ cfu/mL for at least 16 hours at 37° C. The MIC is reported in units of μg/mL.

Bacterial cultures were grown by inoculating Mueller-Hinton Broth (MHB) with 3 or 4 representative colony (typical size, color, and shape) from a bacterial strain grown on a LB agar plate. Inoculated liquid cultures were grown at 37° C. with moderate shaking (200-250 rpm) until bacteria produced a visibly turbid suspension. Bacterial inoculum was approximated by measuring the optical density at 600 nm (0D600) of the bacterial culture against a sterile MHB blank. Typically, OD600=0.10 corresponds to ˜1×10⁸ cfu/mL for S.aureus and OD600 =0.15 corresponds to ˜1×10⁸ cfu/mL for E.coli.

Test compounds were prepared as stock solutions in vehicle at a concentration 10 times the highest concentration to be tested for antibacterial activity (usually 320 μg/mL stock solutions). 2x serial dilutions in vehicle (at 10× the final test concentrations) of the test compounds were prepared from the test compound stock solutions in a non-binding 96 well plate.

In a sterile clear flat bottom 96 well plate, 15 μL of each test compound dilution was added to 135 μL of ˜5×10⁵ cfu/mL bacterial culture. Each concentration was tested in duplicate. Each experiment contained positive control wells for bacterial growth (i.e., no test compound added to bacteria) and sterility controls wells (i.e., sterile MHB and no test compounds added). 96 well plates containing bacterial cultures and test compounds were incubated at 37° C. for 16-20 hours with shaking at 200 rpm.

After incubation, the MIC of each test compound was determined by measuring the OD600 of each well using a plate reader. Wells with OD600 >0.08 were considered to have bacterial growth and wells with OD600<0.08 were considered to have no bacterial growth. The lowest concentration of test agent able to inhibit bacterial growth (0D600<0.08 after 16-20 hours) was defined as the MIC.

17b. C. difficile and B. fragilis

The broth microdilution susceptibility assay followed the procedure described by the Clinical and Laboratory Standards Institute (CLSI) in Methods for Antimicrobial Susceptibility Testing of Anaerobic Bacteria; Approved Standard—Eighth Edition, CLSI document M11-A8 and employed automated liquid handlers (Biomek 2000 and Biomek FX, Beckman Coulter, Fullerton Calif.) to conduct serial dilutions and liquid transfers. The wells in columns 2-12 in standard 96-well microdilution plates (Costar) were filled with 150 μL of 0.01% acetic acid containing 0.2% bovine serum albumin at 10X the final concentration. This resulted in a final concentration of 0.001% acetic acid and 0.02% bovine serum albumin in each well. These would become the ‘mother plate’ from which ‘daughter’ or test plates would be prepared. The test compounds (300 μL at 10× the desired top concentration in the test plates) were dispensed into the appropriate well in Column 1 of the mother plates. The Biomek 2000 was used to make serial 2-fold dilutions through Column 11 in the “mother plate”. The wells of Column 12 contained no test compound, representing the organism growth control wells.

Using a multi-channel pipette, the daughter plates were filled with 170 μL of Supplemented Brucella Broth per well (BectonDickinson; Sparks, Mar.; Catalog 211088, Lot 3182288). This broth was supplemented with 5 mg/mL hemin (Sigma; Lot SLBC4685V), 1 mg/mL vitamin K (Sigma; Lot 108K1088), and 5% Laked Horse blood (Cleveland Scientific; Lot 291960). Another corresponding set of Costar plates were prepared using Reinforced Clostridial Broth (Hi-Media; Lot 0000186925) as the growth medium. The daughter plates were prepared with the Biomek FX, transferring 20 μL of a test compound solution from each well of the mother plate to each corresponding well of each daughter plate in a single step.

Standardized inocula of the organisms were prepared per CLSI methods. Bacterial suspensions were prepared in supplemented Brucella Broth to equal the turbidity of a 0.5 McFarland standard. The 0.5 McFarland suspensions were further diluted 1:10 in broth. The inoculum was dispensed into sterile reservoirs (Beckman Coulter), and the inoculum was transferred by hand in the Bactron Anaerobe chamber so that inoculation took place from low to high drug concentration. 10 μL of inoculum was delivered into each well. Thus, the wells of the daughter plates ultimately contained 170 μL of broth, 20 μL of a test compound solution, and 10 μL of inoculum. For the comparator drugs, the wells contained 185 μL of media, 5 μL of drug and 10 μL of inoculum. For each isolate, a separate row contained 10 μL of inoculum, 20 μL of solvent, and 170 μL of media (no test compound) to confirm that the low levels of acetic acid would not inhibit growth.

Plates were stacked and placed in an anaerobic box with GasPak sachets (BectonDickinson; Lot 5327518), covered with a lid on the top plate, and incubated at 35° -37° C. The microplates were viewed from the bottom using a plate viewer after 44-48 hours. For each mother plate, an un-inoculated solubility control plate was observed for evidence of test compound precipitation. The MIC was read and recorded as the lowest concentration of a test compound that inhibited visible growth of the organism. The results obtained from Examples 17a and 17b are summarized in Table 4 below.

TABLE 4

| | MIC (μg/mL) | | | | MIC ratio |
|---|---|---|---|---|---|
| Compound # | S. aureus | C. difficile | E. coli | B. fragilis | Coli/Staph |
| 1 | 0.06 | <0.03 | 4 | >32 | 67 |
| 2 | 0.09 | 0.06 | >32 | >32 | >356 |
| 3 | 0.09 | N/A | >32 | N/A | >356 |
| 4 | 0.13 | 0.06-0.12 | 8 | >32 | 62 |
| 5 | 0.13 | <0.03-0.06 | 10 | >32 | 77 |
| 6 | 0.13 | 0.03 | 8 | >32 | 62 |
| 7 | 0.13 | ≤0.03-0.06 | 32 | >32 | 246 |
| 8 | 0.13 | 0.06 | 4 | >32 | 31 |
| 9 | 0.13 | 0.12 | 16 | >32 | 123 |
| 10 | 0.19 | N/A | 16 | N/A | 84 |
| 11 | 0.25 | 0.12 | 16 | >32 | 64 |
| 12 | 0.25 | 0.25 | 32 | >32 | 128 |
| 13 | 0.25 | N/A | 16 | N/A | 64 |
| 14 | 0.25 | 0.06-0.12 | 4 | >32 | 16 |
| teixobactin | 0.19 | <0.03 | 8 | >32 | 42 |
| Lys10- | 0.25 | 0.12 | 16 | >32 | 64 |
| teixobactin* | | | | | |
| Arg10- | 0.25-0.5 | 0.12 | >16 | >32 | >64 |
| teixobactin | | | | | |
| 70 | 0.03-0.06 | 0.015-0.03 | >16 | >32 | >530 |
| 71 | 0.03-0.06 | 0.03-0.06 | >16 | >32 | >530 |
| 72 | 0.12-0.25 | 0.25-0.5 | >16 | >32 | >128 |
| 74 | 0.06 | 0.06-0.12 | >16 | >32 | >530 |
| 75 | 0.06 | 0.015-0.03 | >16 | >32 | >530 |

*Lys10-teixobactin by is a teixobactin derivative in which the 10^{th} amino acid is replaced Lys.

### 17c: MIC90 determination for S.aureus, S.epidermidis, S.pneumoniae, S.pyoenes, E. faecalis, and E.faecium

The broth microdilution susceptibility assay followed the procedure described by the Clinical and Laboratory Standards Institute (CLSI) in CLSI document M7-A10.

The following bacteria isolates were tested:

| species | isolates tested |
|---|---|
| S. aureus | ACC00040, ACC00041, ACC00044, |
| (14) | ACC00054, ACC00052, ATCC33591, |
| | ACC00061, ATCC43300, ATCC13709, |
| | ATCC29213, ATCC25923, ACC00017, |
| | ACC00058, ACC00059 |
| S. epidermidis | ACC00292, ACC00067, ACC00293, |
| (5) | ACC00588, ATCC35984 |
| S. pneumoniae | ACC00126, ACC00107, BAA1407, |
| (8) | ACC00117, ACC00104, ACC00236, |
| | ATCC49619, ACC00113, |
| S. pyogenes (2) | ACC00074, ACC00075 |
| E. faecalis | ATCC29212, ACC00404, ACC00405, |
| (5) | ACC00408, ACC00409 |
| E. faecium (3) | ACC00420, ACC00626, ACC00627 |

S.aureus, S.epidermidis, E.faecalis, and E.faecium colonies were grown on TSA (Tryptone Soya Agar; cat.N. P05012A -OXOID) S.pneumoniae and S.pyogenes colonies were grown on TSASB ((TSA) Agar +5% Sheep Blood; cat.N. PB5012A -OXOID). An inoculum was prepared by making a direct saline suspension of isolated colonies selected from an 18 to 24 hours agar plate incubated at 35±2 ° C. in ambient air. The suspension was adjusted to achieve a turbidity equivalent to a 0.5 McFarland turbidity standard (1 to 2 ×108 Colony Forming Units (CFU)) and diluted 200 fold within 15 minutes in broth. (S.aureus, S.epidermidis, E.faecalis, and E.faecium were diluted in CAMHB: Mueller Hinton Broth 2, Cation-Adjusted (cat. N. 90922 Fluka); S.pneumoniae and S.pyogenes were diluted in CAMHB +2.5% lysed horse blood. All broth contained 0.002% polysorbate 80.

96-well plates were prepared containing 1 μL of the test compound (at 100× desired test concentration in 100% DMSO); compounds were tested at 8 final concentrations, i.e. 16 μg/mL, 8 μg/mL, 4 μg/mL, 2 μg/mL, 1 μg/mL, 0.5 μg/mL, 0.25 μg/mL, and 0.125 μg/mL. 100 μL of the broth dilution was dispensed in each well plate to have a final bacterial concentration in plate of ˜5×10⁵ CFU/mL. The plates were incubated at 35±2° C. in ambient air for 20 hours.

The MIC for each individual isolate was defined as the lowest concentration of test compound agent that completely inhibited growth of the organism in the microdilution wells as detected by the unaided eye. The MIC90 for each species was determined as the concentration required to completely inhibit growth of 90% of the tested isolates of that species. The MIC90 values of Compound 5 and 70-75 against the above six bacteria were obtained and summarized in Table 5.

TABLE 5

| Compound | S. Aureus | S. epidermidis | S. pneumoniae | S. pyogenes | E. faecalis | E. faecium |
|---|---|---|---|---|---|---|
| # | (14) MIC90 | (MIC90) (5) | (8) MIC90 | (2) MIC90 | (5) MIC90 | (3) MIC90 |
| 5 | 0.25 | ≤0.12 | ≤0.12 | ≤0.12 | 0.5 | 0.5 |
| 70 | ≤0.12 | ≤0.12 | ≤0.12 | ≤0.12 | 0.25 | ≤0.12 |
| 71 | ≤0.12 | ≤0.12 | ≤0.12 | ≤0.12 | 0.5 | 0.5 |
| 72 | 0.25 | ≤0.12 | ≤0.12 | ≤0.12 | 0.5 | 0.5 |
| 73 | 0.5 | 0.25 | ≤0.12 | ≤0.12 | 0.5 | 0.25 |
| 74 | ≤0.12 | ≤0.12 | ≤0.12 | ≤0.12 | 1 | 0.5 |
| 75 | ≤0.12 | ≤0.12 | ≤0.12 | ≤0.12 | ≤0.12 | ≤0.12 |
| teixobactin | ≤0.12 | ≤0.12 | ≤0.12 | ≤0.12 | 0.5 | 0.5 |
| [Arg10] | 0.5 | 0.5 | 0.25 | 0.25 | 2 | 2 |
| teixobactin | | | | | | |
| [Lys10] | 0.5 | 0.5 | ≤0.12 | 0.25 | 2 | 1 |
| teixobactin | | | | | | |

As shown in Table 5, Compounds 5 and 70-75 of the invention showed lower MIC90 than the [Arg10]teixobactin or [Lys10]teixobactin. Although Compound 5 and 70-75 showed MIC90 similar to natural compound teixobactin, they are less cytotoxicity than teixobactin as demonstrated in Example 18 below.

### Example 18: Mammalian Cell Line Cytotoxicity Assay

Compounds were tested for cytotoxicity to a mammalian cell line HepG2 (Human hepatocyte carcinoma from ECACC (85011430)). Hep G2 cells were seeded into 96-well plates, 7500 cells/well in 100 μl medium (Minimum Essential Medium (Gibco 21090) supplemented with 2 mM L-Glutamine, 1% non-essential amino acids and 10% fetal bovine serum) and incubated for 20 hours at 37° C., 5% CO₂. 100 μL of fresh medium containing a test compound or DMSO (for control) at 2× final concentration were added and incubated for 48 hours at 37° C., 5% CO₂. The final concentration of DMSO in plate was 1%. At the end of this incubation, 20 μl MTT (Thiazolyl Blue Tetrazolium Bromide) solution 5 mg/ml in H₂O (0.5 mg/ml final in well) was added to all wells. Cells were incubated with MTT for 4 hours at 37° C., 5% CO₂. After incubation, medium containing MTT was removed; 200 μl of DMSO was added in each well and plates were put on a shaker for 5 minutes. Absorbance was read on a SpectraMax plate reader at 570 nm. Specific absorbance (Specific A570) was calculated by subtracting the mean absorbance values of the blank wells (no HepG2 cells) from each well. Control values were determined from wells containing cells and treated with medium+1% DMSO only (no test compound). % viability was calculated as:

(Specific A570_{test compound}/Specific A570_{control})×100.

The results are summarized in Table 6 below. As shown in Table 6, compounds of the invention showed less cytotoxicity than the natural compound teixobactin.

TABLE 6

| | Cell viability |
|---|---|
| Compound # | (% at 100 uM) |
| 5 | 117.2 |
| 70 | 114.3 |
| 71 | 92.0 |
| 72 | 114.6 |
| 73 | 113.4 |
| 74 | 136.3 |
| 75 | 119.2 |
| teixobactin | 56.3 |
| [Arg10]teixobactin | 115.4 |
| [Lys10]teixobactin | 95.6 |

Other embodiments are within the scope of the following claims.

1. A compound of formula (I) or a pharmaceutically acceptable salt thereof: 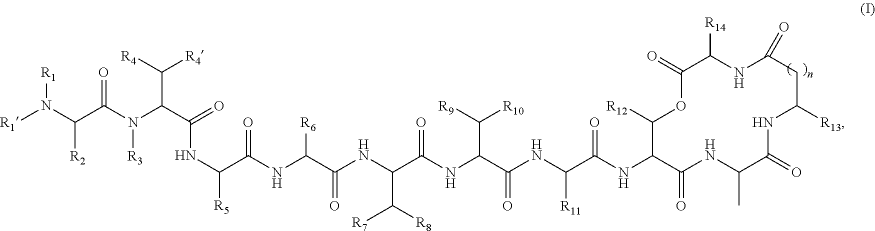
wherein
n is 0 or 1;
each of R₁ and R₁′, independently, is H, C₁-C₆ alkyl, C(O)—Rₐ, or C(O)O—Rₐ, in which Rₐ is C₁-C₆ alkyl optionally substituted with NH₂, aryl, or heteroaryl;
R₂ is C₁-C₆ alkyl optionally substituted with aryl or heteroaryl, in which the aryl or heteroaryl group is optionally substituted with halo, NH₂, C₁-C₆ alkyl, or C₁-C₆ alkoxy;
R₃ is H, C₁-C₆ alkyl, or C(O)—R_{b}, in which R_{b} is C₁-C₆ alkyl;
each of R₄ and R₄′, independently, is H, C₁-C₆ alkyl, aryl, or heteroaryl;
R₅ is C₁-C₆ alkyl optionally substituted with OH, NH—R_{c}, aryl, or heteroaryl, or aryl optionally substituted with C₁-C₆ alkyl, in which R_{c} is H, C(O)O—R_{c}′, or —SO₂-phenyl optionally substituted with C₁-C₆ alkyl, R_{c} ′ being C₁-C₆ alkyl or C₁-C₆ alkenyl;
R₆ is C₁-C₆ alkyl optionally substituted with C(O)—NH(R_{d}), NH(R_{d}), NHC(O)—R_{d}, aryl, or heteroaryl, in which each R_{d}, independently, is H, aryl, heteroaryl, or C₁-C₆ alkyl optionally substituted with aryl;
R₇ is H, C₁-C₆ alkyl, aryl, or heteroaryl;
R₈ is H, C₁-C₆ alkyl, aryl, or heteroaryl;
R₉ is H, C₁-C₆ alkyl, aryl, or heteroaryl;
R₁₀ is H, C₁-C₆ alkyl, aryl, or heteroaryl;
R₁₁ is C₁-C₆ alkyl optionally substituted with OH, NH₂, aryl, or heteroaryl;
R₁₂ is H or C₁-C₆ alkyl;
R₁₃ is C₁₃-C₆ alkyl optionally substituted with heteroaryl, NH(=NH)NH(Rₑ), NH(=O)NH(R_{c}), N(Rₑ)₂, N(Rₑ)₃⁺, COOL, COO-NH(CH₂)₂N(Rₑ)₂, or aryl optionally substituted with NH(=NH)NH(Rₑ), in which each Rₑ, independently, is H, NO₂, C₁-C₆ alkyl optionally substituted with aryl, or C(O)—Rₑ′, Rₑ′ being C₁-C₆ alkyl; and
R₁₄ is C₁-C₆ alkyl optionally substituted with NH₂;
provided that the compound is not

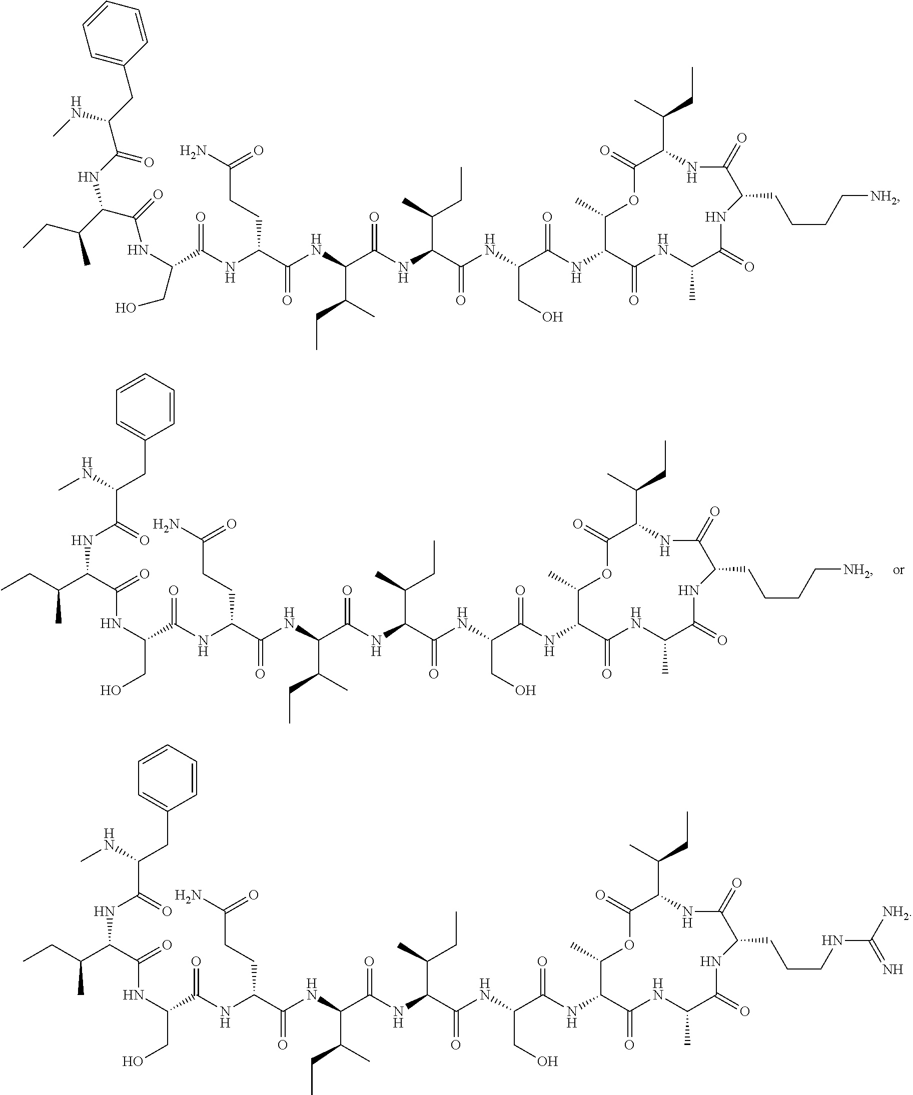

2. The compound of claim 1, wherein the compound is of formula (II):

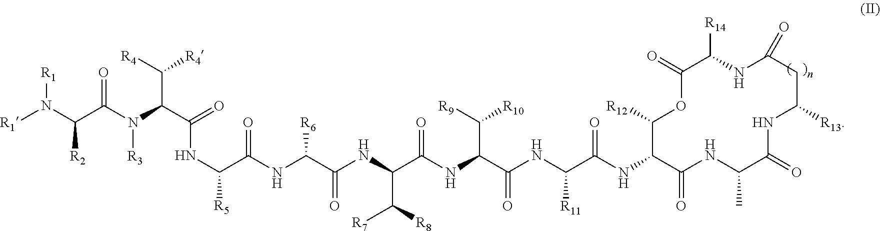

3. The compound of claim 1, wherein n is 0.

4. The compound of claim 1, wherein:
R₁ is H or C₁-C₆ alkyl, and R₁′ is H; or
R₂ is C₁-C₆ alkyl substituted with phenyl, in which the phenyl group is optionally substituted with halo; or
R₃ is H or C(O)—R_{b}, in which R_{b} is C₁-C₆ alkyl; or
R₄ is H, C₁-C₆ alkyl, or heteroaryl, and R₄′ is H or C₁-C₆ alkyl; or
R₅ is aryl, or C₁-C₆ alkyl substituted with OH, NH₂, or heteroaryl; or
R₆ is C₁-C₆ alkyl substituted with C(O)NH₂; or
each of R₇, R₈, R₉, R₁₀, R₁₂, and R₁₄ is C₁-C₆ alkyl; or
R₁₁ is C₁-C₆ alkyl substituted with OH; or
R₁₃ is C₁-C₆ alkyl substituted with NH(=NH)NH(Rₑ) or N(Rₑ)₂, in which each Rₑ, independently, is H or C₁-C₆ alkyl.

5-12. (Canceled)

13. A compound of formula (II) or a pharmaceutically acceptable salt thereof: 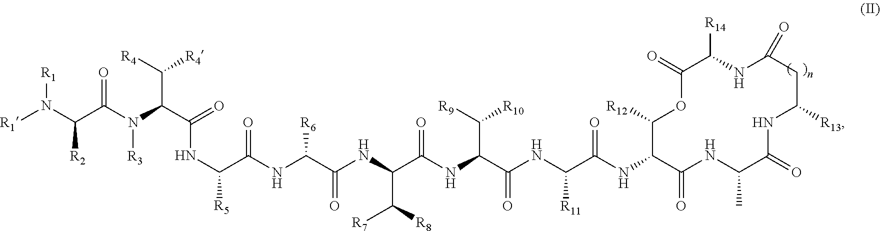
wherein
n is 0 or 1;
each of R₁ and R₁′, independently, is H, C₁-C₆ alkyl, C(O)—Rₐ, or C(O)O-Rₐ, in which Rₐ is C₁-C₆ alkyl optionally substituted with NH₂, aryl, or heteroaryl;
R₂ is C₁-C₆ alkyl optionally substituted with aryl or heteroaryl, in which the aryl or heteroaryl group is optionally substituted with halo, NH₂, C₁-C₆ alkyl, or C₁-C₆ alkoxy;
R₃ is H, C₁-C₆ alkyl, or C(O)—R_{b}, in which R_{b} is C₁-C₆ alkyl;
each of R₄ and R₄′, independently, is H, C₁-C₆ alkyl, aryl, or heteroaryl;
R₅ is C₁-C₆ alkyl optionally substituted with OH, NH—Rₑ, aryl, or heteroaryl, or aryl optionally substituted with C₁-C₆ alkyl, in which R_{c} is H, C(O)O—R_{c}′, or —SO₂-phenyl optionally substituted with C₁-C₆ alkyl, R_{c}′ being C₁-C₆ alkyl or C₁-C₆ alkenyl;
R₆ is C₁-C₆ alkyl optionally substituted with C(O)—NH(R_{d}), NH(R_{d}), NHC(O)—R_{d}, aryl, or heteroaryl, in which each R_{d}, independently, is H, aryl, heteroaryl, or C₁-C₆ alkyl optionally substituted with aryl;
R₇ is H, C₁-C₆ alkyl, aryl, or heteroaryl;
R₈ is H, C₁-C₆ alkyl, aryl, or heteroaryl;
R₉ is H, C₁-C₆ alkyl, aryl, or heteroaryl;
R₁₀ is H, C₁-C₆ alkyl, aryl, or heteroaryl;
R₁₁ is C₁-C₆ alkyl optionally substituted with OH, NH₂, aryl, or heteroaryl;
R₁₂ is H or C₁-C₆ alkyl;
R₁₃ is C₁, C₂, or C₄-C₆ alkyl optionally substituted with heteroaryl, NH(=NH)NH(Rₑ), NH(=O)NH(Rₑ), COORₑ, COO—NH(CH₂)₂N(Rₑ)₂, or aryl optionally substituted with NH(=NH)NH(Rₑ), in which each Rₑ, independently, is H, NO₂, C₁-C₆ alkyl optionally substituted with aryl, or C(O)—Rₑ′, Rₑ′ being C₁-C₆ alkyl; and
R₁₄ is C₁-C₆ alkyl optionally substituted with NH_{2.}

14. The compound of claim 13, wherein n is 0.

15. The compound of claim 13, wherein:
R₁ is C₁-C₆ alkyl and R₁′ is H; or
R₂ is C₁-C₆ alkyl substituted with phenyl; or
R₃ is H; or
R₄ is C₁-C₆ alkyl and R₄′ is C₁-C₆ alkyl; or
R₅ is C₁-C₆ alkyl substituted with OH, NH₂, or heteroaryl; or
R₆ is C₁-C₆ alkyl substituted with C(O)NH₂; or wherein each of R₇, R₈, R₉, R₁₀, R₁₂, and
R₁₄ is C₁-C₆ alkyl; or
R₁₁ is C₁-C₆ alkyl substituted with OH; or
R₁₃ is C₁-C₆ alkyl substituted with NH(=NH)NH(Rₑ), in which Rₑ is H or C₁-C₆ alkyl.

16-23. (Canceled)

24. The compound of claim 13, wherein the compound is

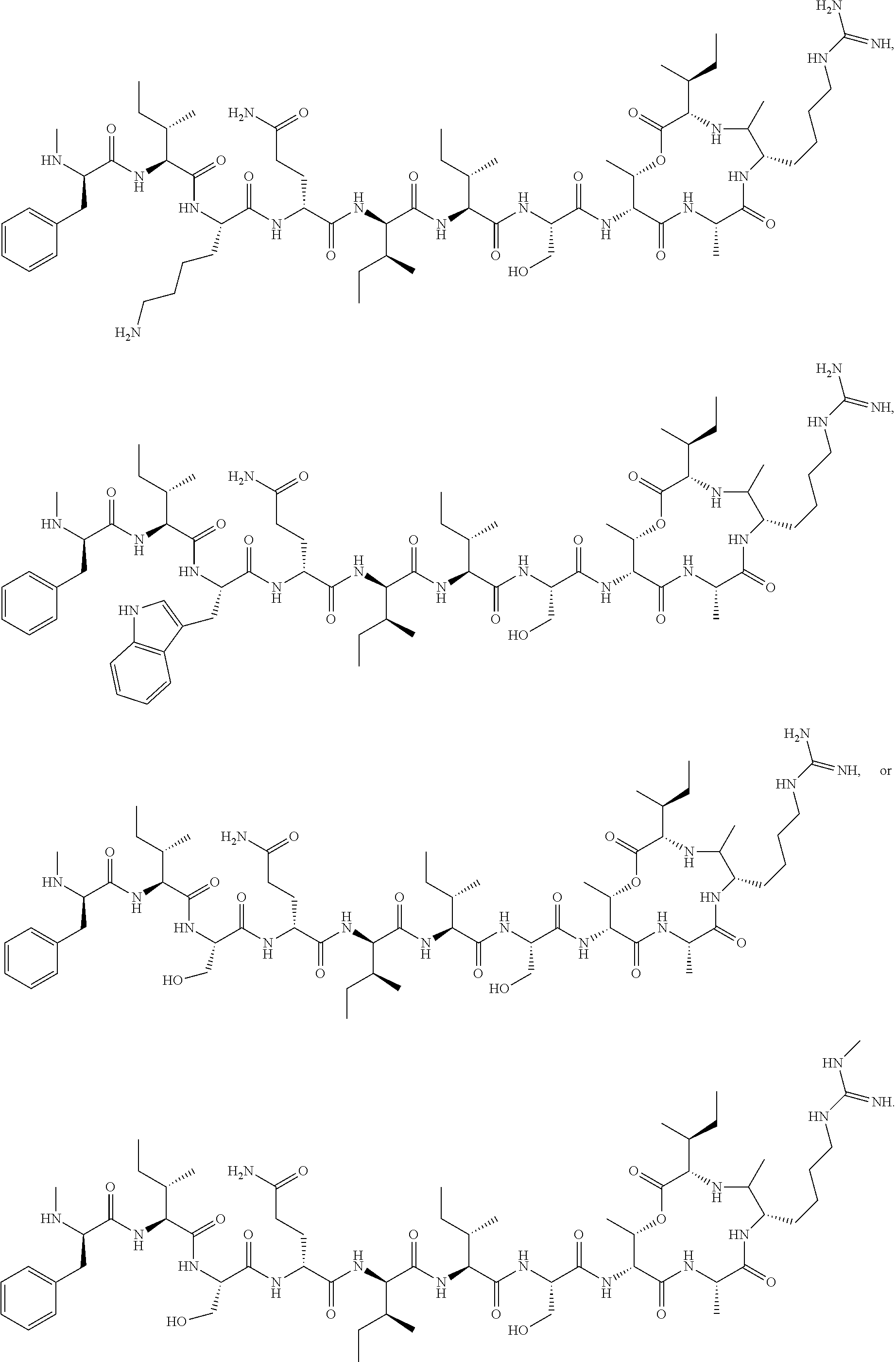

25. A compound of formula (II) or a pharmaceutically acceptable salt thereof: 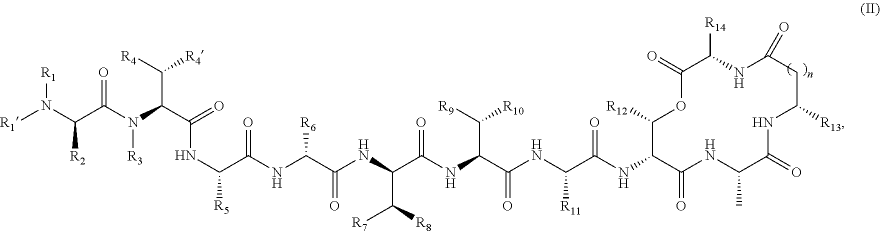
wherein
n is 0 or 1;
each of R₁ and R₁′, independently, is H, C₁-C₆ alkyl, C(O)—Rₐ, or C(O)O—Rₐ, in which Rₐ is C₁-C₆ alkyl optionally substituted with NH₂, aryl, or heteroaryl;
R₂ is C₁-C₆ alkyl optionally substituted with aryl or heteroaryl, in which the aryl or heteroaryl group is optionally substituted with halo, NH₂, C₁-C₆ alkyl, or C₁-C₆ alkoxy;
R₃ is H, C₁-C₆ alkyl, or C(O)—R_{b}, in which R_{b} is C₁-C₆ alkyl; each of R₄ and R₄′, independently, is H, C₁-C₆ alkyl, aryl, or heteroaryl;
R₅ is C₁-C₆ alkyl optionally substituted with OH, NH—R_{c}, aryl, or heteroaryl, or aryl optionally substituted with C₁-C₆ alkyl, in which R_{c} is H, C(O)O—R_{c}′, or —SO₂-phenyl optionally substituted with C₁-C₆ alkyl, R_{c}′ being C₁-C₆ alkyl or C₁-C₆ alkenyl;
R₆ is C₁-C₆ alkyl optionally substituted with C(O)—NH(R_{d}), NH(R_{d}), NHC(O)—R_{d}, aryl, or heteroaryl, in which each R_{d}, independently, is H, aryl, heteroaryl, or C₁-C₆ alkyl optionally substituted with aryl;
R₇ is H, C₁-C₆ alkyl, aryl, or heteroaryl;
R₈ is H, C₁-C₆ alkyl, aryl, or heteroaryl;
R₉ is H, C₁-C6 alkyl, aryl, or heteroaryl;
R₁₀ is H, C₁-C₆ alkyl, aryl, or heteroaryl;
R₁₁ is C₁-C₆ alkyl optionally substituted with OH, NH₂, aryl, or heteroaryl;
R₁₂ is H or C₁-C₆ alkyl;
R₁₃ is C₁, C₂, C₅ or C6 alkyl optionally substituted with heteroaryl, NH(=NH)NH(Rₑ), NH(=O)NH(Rₑ), N(Rₑ)₂, N(Rₑ)₃⁺, COORₑ, COO—NH(CH₂)₂N(Rₑ)₂, or aryl optionally substituted with NH(=NH)NH(Rₑ), in which each Rₑ, independently, is H, NO₂, C₁-C₆ alkyl optionally substituted with aryl, or C(O)—Rₑ′, Rₑ′ being C₁-C₆ alkyl; and
R₁₄ is C₁-C₆ alkyl optionally substituted with NH_{2.}

26. The compound of claim 25, wherein n is 0.

27. The compound of claim 25, wherein:
R₁ is C₁-C₆ alkyl and R₁′ is H; or .
R₂ is C₁-C₆ alkyl substituted with phenyl; or
R₃ is H; or
R₄ is C₁-C₆ alkyl and R₄′ is C₁-C₆ alkyl; or
R₅ is C₁-C₆ alkyl substituted with OH; or
R₆ is C₁-C₆ alkyl substituted with C(O)NH₂; or
each of R₇, R₈, R₉, R₁₀, R₁₂, and R₁₄ is C₁-C₆ alkyl; or
R₁₁ is C₁-C₆ alkyl substituted with OH; or
R₁₃ is C₁, C₂, C₅ or C₆ alkyl substituted with NH_{2.}

28-35. (canceled)

36. The compound of claim 25, wherein the compound is

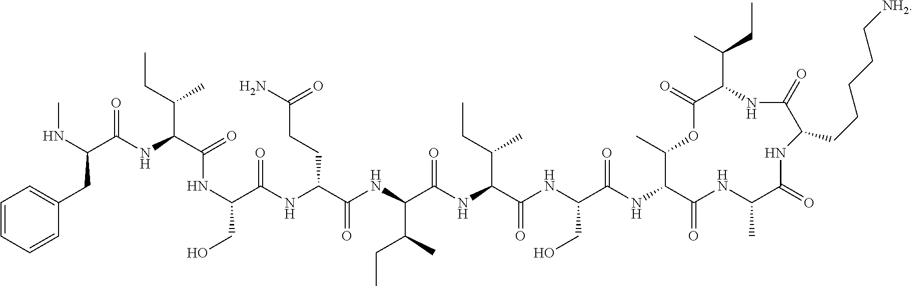 [SMILES: CC[C@H](C)[C@H](NC(=O)[C@@H](Cc1ccccc1)NC)C(=O)N[C@@H](CO)C(=O)N[C@H](CCC(N)=O)C(=O)N[C@@H](C(=O)N[C@H](C(=O)N[C@@H](CO)C(=O)N[C@H]1C(=O)N[C@@H](C)C(=O)N[C@@H](CCCCCN)C(=O)N[C@@H]([C@@H](C)CC)C(=O)O[C@H]1C)[C@@H](C)CC)[C@@H](C)CC]

37. The compound of claim 1, wherein the compound is a compound of formula (III) or a pharmaceutically acceptable salt thereof: 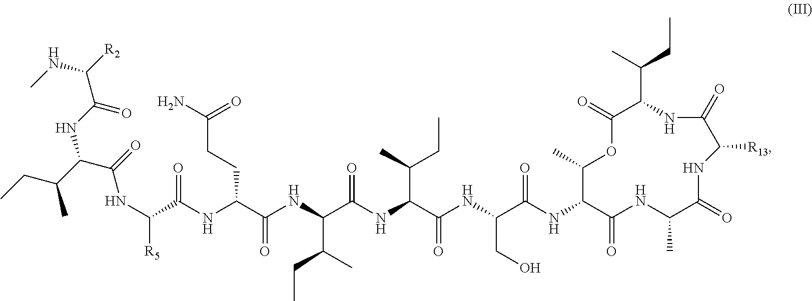
wherein
R₂ is C₁-C₆ alkyl optionally substituted with aryl or heteroaryl, in which the aryl or heteroaryl group is optionally substituted with halo, NH₂, C₁-C₆ alkyl, or C₁-C₆ alkoxy;
R₅ is C₁-C₆ alkyl optionally substituted with OH, NH—R, aryl, or heteroaryl, or aryl optionally substituted with C₁-C₆ alkyl, in which Rₐ is H, C(O)O—R_{c}′, or —SO₂-phenyl optionally substituted with C₁-C₆ alkyl, R_{c}′ being C₁-C₆ alkyl or C₁-C₆ alkenyl; and
R₁₃ is C₁-C₆ alkyl optionally substituted with heteroaryl, NH(=NH)NH(Rₑ), NH(=O)NH(Rₑ), N(Rₑ)₂, N(Rₑ)₃⁺, COORₑ, COO—NH(CH₂)₂N(Rₑ)₂, or aryl optionally substituted with NH(=NH)NH(Rₑ), in which each Rₑ, independently, is H, NO₂, C₁-C₆ alkyl optionally substituted with aryl, or C(O)—R_{c}′, Rₑ′ being C₁-C₆ alkyl.

38. The compound of claim 37, wherein:
R₁ is C₁-C₆ alkyl substituted with phenyl, chlorophenyl, methoxyphenyl, or naphthyl; or
R₂ is C₁-C₆ alkyl optionally substituted with OH, NH—R_{c}, indolyl, naphthyl, in which Rₐ is H, C(O)O-allyl, or —SO₂-tosyl.

39. (canceled)

40. The compound of claim 37, wherein R3 is C₁-C₆ alkyl optionally substituted with NH(=NH)NH₂, NHCH₂Ph, or phenyl substituted with NH(=NH)NH₂.

41. The compound of claim 37, wherein the compound is

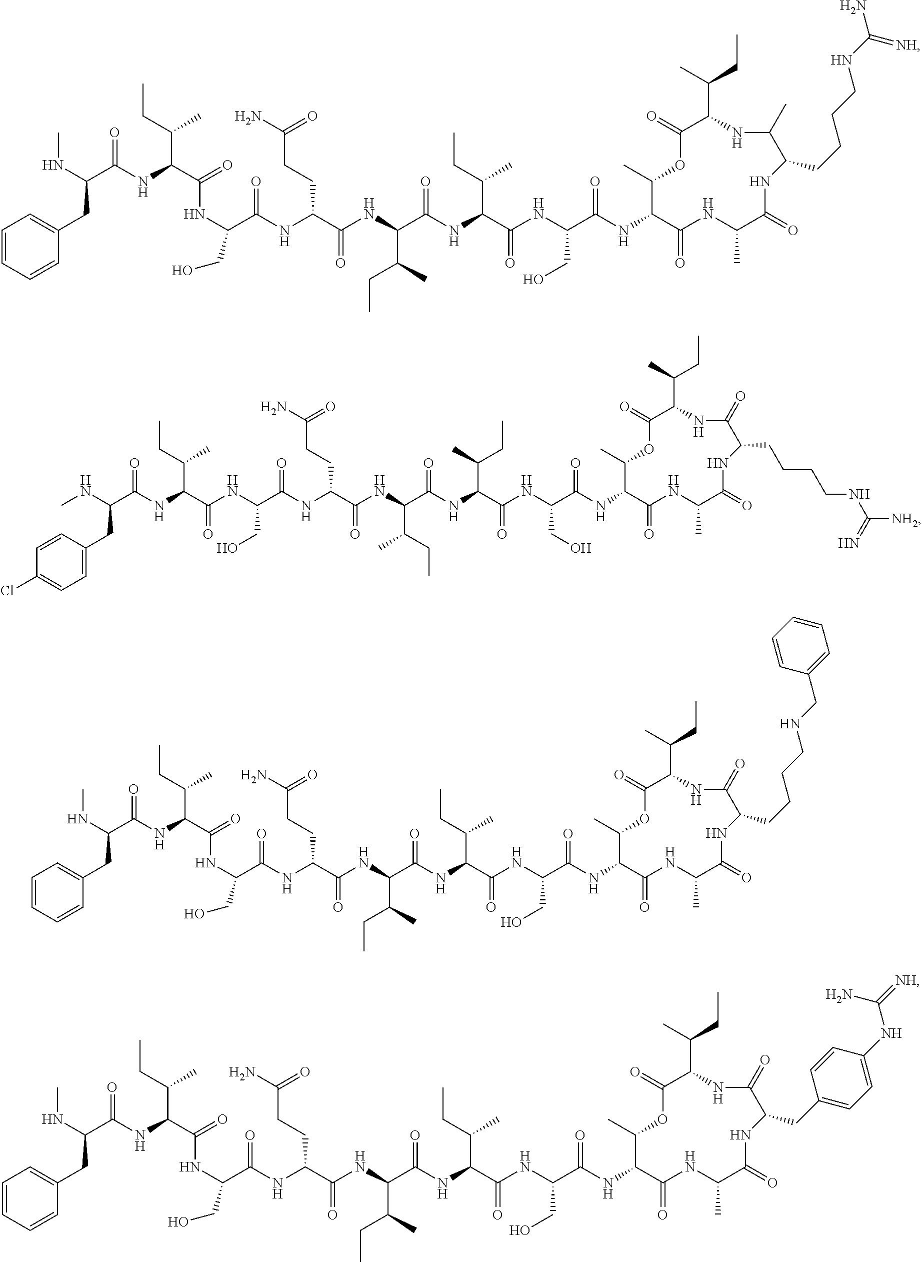

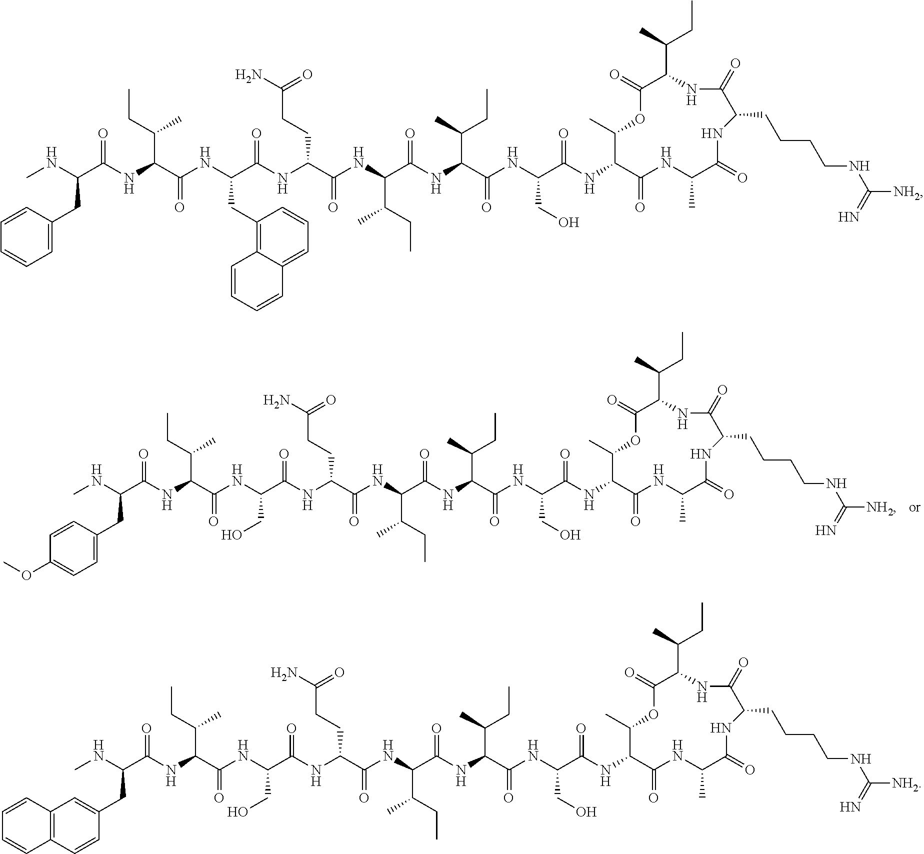

42. A pharmaceutical composition, comprising the compound claim 1 and a pharmaceutically acceptable carrier.

43. A method of treating bacterial infection, comprising administering to a patient in need thereof an effective amount of the pharmaceutical composition of claim 42.

44. The method of claim 43, wherein the bacterial infection is a gram-positive bacterial infection.

45. The method of claim 44, wherein the bacterial infection is Clostridium difficile infection or Staphylococcus aureus infection.

46-52. (canceled)
